SEQUENCE LISTING (3 sequences)

SEQ ID NO: 1 - DNA, 25 bp, Artificial Sequence
  human beta-actin DNA forward primer
tcacccacactgtgcccatctacga

SEQ ID NO: 2 - DNA, 25 bp, Artificial Sequence
  human beta-actin DNA reverse primer
cagcggaaccgctcattgccaatgg

SEQ ID NO: 3 - DNA, 26 bp, Artificial Sequence
  human beta-actin DNA probe
  FAM (1)..(1)
  BHQ (26)..(26)
atgccctcccccatgccatcctgcgt

(19) United States
(12) Patent Application Publication
(10) Pub. No.: US 2022/0349014 A1
(43) Pub. Date: Nov. 3, 2022

(54) MULTIMODAL ANALYSIS OF STABILIZED CELL-CONTAINING BODILY FLUID SAMPLES

(71) Applicant: Qiagen GmbH, Hilden (DE)

(72) Inventors: Anna BABAYAN, Hilden (DE); Andrea HUXHOLD (née ULLIUS), Hilden (DE); Daniel GRÖLZ, Hilden (DE); Kalle GÜNTHER, Hilden (DE)

(21) Appl. No.: 17/762,565

(22) PCT Filed: Sep. 24, 2020
(86) PCT No.: PCT/EP2020/076801

§ 371 Date: Mar. 22, 2022

(30) Foreign Application Priority Data
Sep. 24, 2019 (EP) 19199283.3

(51) Int. Cl. C12Q 1/6886; C12Q 1/6806; A01N 1/02

(52) U.S. Cl. CPC C12Q 1/6886; C12Q 1/6806; A01N 1/0226; C12Q 1/6851

(57) ABSTRACT

A method for stabilizing and isolating multiple biological targets comprised in a cell-containing bodily fluid, said method comprising (A) contacting a cell-containing bodily fluid with a stabilizing composition comprising one or more of the following stabilizing agents: (a) at least one primary, secondary or tertiary amide, (b) at least one poly(oxyethylene) polymer, and/or (c) at least one apoptosis inhibitor, thereby providing a stabilized cell-containing bodily fluid sample; (B) keeping the stabilized cell-containing bodily fluid sample for a stabilization period; and (C) processing the stabilized cell-containing bodily fluid sample in order to enrich three or more biological targets selected from the group consisting of —a cell subpopulation, —extracellular nucleic acids, —extracellular vesicles and —intracellular nucleic acids from the stabilized cell-containing bodily fluid. The method is advantageous and enables the multimodal analyses of different biological targets from a single stabilized cell-containing body fluid sample.

### FIELD OF THE DISCLOSURE

Provided are liquid biopsy based methods and workflows for the analysis of different biological targets of interest from a single stabilized cell-containing bodily fluid sample.

### BACKGROUND

Liquid biopsy (LB) as analysis of biological targets (e.g. cells, proteins, nucleic acids) in human body liquids (in particular blood, urine, saliva, liquor, etc.) is a powerful tool for companion diagnostics in clinical practice. Important liquid biopsy analytes, also referred to herein as biological targets, include rare cells, extracellular nucleic acids, extracellular vesicles, intracellular nucleic acids as well as specific cell subpopulations. Liquid biopsy in cancer and prenatal testing have raised the most interest and some of the currently existing tests have been already introduced into routine patient care.

Solid tumors and hematologic malignancies are known to shed biological materials into the systemic circulation. These include cells (circulating tumor cells, also referred to as CTCs) and extracellular vesicles (also referred to as EVs) such as exosomes and other types of sub-cellular membrane vesicles. Free circulating nucleic acids are also known to contain cancer-related information, e.g. on mutations. These biological materials exist in easily accessible bodily fluids, such as peripheral whole blood, peritoneal or pleural effusions, and carry molecular information, including proteins, nucleic acids and lipids. The molecular information provided by these circulating biological materials can be correlated to for example prognosis, therapy response, relapse or therapy resistance mechanisms. There is a high interest in the prior art towards these biological targets for minimally invasive testing. They present significant advantages to circumvent challenges of biopsies and can be easily and repeatedly obtained to provide a minimally invasive reflection of tumor molecular information. It is accepted in the art that extracellular nucleic acids, extracellular vesicles or circulating tumor cells can provide valuable diagnostic, prognostic, predictive and monitoring information. This information can be used e.g. by analyzing biomarkers comprised therein. A biomarker is a biological molecule that is measurable in the biological sample to be analyzed, and which either alone or in combination with other biomarkers can be an indicator of some clinically significant condition. Biomarkers can be e.g. diagnostic, surrogate, prognostic and/or predictive. A biomarker may be e.g. a nucleic acid (e.g. a DNA or RNA molecule) or a protein.

Blood is the most prominent material source for liquid biopsy. Cell-based liquid biopsy tests often rely on the analysis of a target cell population, such as CTCs in cancer (further examples are endothelial cells in cancer, diabetes, cardio-vascular or acute kidney diseases, foetal cells in prenatal testing, organ-specific cells in transplantology) (see e.g. Pantel et al, Nat Rev Clin Oncol, February 2019; Neumann et al., Comput Struct Biotechnol J, 2018, Vol. 16: 190-195; Lehmann-Werman et al., Proc Natl Acad Sci USA. 2016 Mar. 29, Vol. 113(13):E1826-34; Snyder et al., Proc Natl Acad Sci USA, 2011 Apr. 12, Vol. 108(15):6229-34).

CTCs detach from primary or metastatic tumor of a cancer patient and can be found in blood. These cells represent a rare cell population: 1-10 CTCs can be found in a background of 10⁶-10⁸ blood cells with half-life time in circulation limited to 2.5 hours. CTCs are the seeds of distant metastases. Presence of CTCs in peripheral blood of cancer patients have been introduced and validated as a surrogate marker for overall and disease-free survival and can be used as prognostic, predictive and therapy-guiding biomarker. Besides enumeration, examination of phenotypic, genotypic and transcriptomic features on CTCs provides therapy- and outcome-relevant information. However, CTC analysis is hampered due to 1) low abundance of the CTCs in high background of white blood cells (WBCs) and 2) their short half-life time in the circulatory system. Because of their rarity, CTCs have to be enriched prior to detection/analysis. Multiple existing enrichment methods can be basically separated into label-dependent and label-independent approaches (Joosse et al., EMBO Mol Med, 2015 January, Vol. 7(1):1-11). Whereas the label-dependent methods rely on isolation of target cell population based on biological properties, such as expression of specific antigens on cell surface, label-independent methods utilize physical properties of tumor cells, such as size, density, deformability and other features. Detection of CTCs is possible on cellular level (based on antigen-specific staining of target protein) and molecular level, e.g. based on detection of tumor-relevant transcripts, genomic or epigenomic aberrations.

Another prominent liquid biopsy analyte is extracellular nucleic acids such as circulating cell-free DNA (ccfDNA). A major source for ccfDNA are mono-nucleosomal DNA fragments originating from apoptotic and necrotic cells. Furthermore, extracellular DNA is also present as vesicle-bound apoptotic bodies, microparticles, microvesicles, exosomes or histone/DNA complexes, nucleosomes, and virtosomes. In addition, extracelluar RNA is present inside exosomes and other extracellular vesicles (EVs). In cancer patients a certain proportion of ccfDNA is circulating tumor DNA (ctDNA) originating from tumor cells. Given the tumor-specific aberration on genomic and epigenomic levels, ctDNA can be effectively detected in high background of wild type ccfDNA. Modern technologies (such as digital droplet PCR, BEAMing, next generation sequencing) allow for development and rapid implementation of ccfDNA-based liquid biopsy tests into clinical practice (e.g. cobas EGFR Mutation Test v2, Therascreen KRAS test). A similar concept is implemented in non-invasive prenatal testing and organ rejection in transplantology (relying on detection of rare foetal DNA fragments in background of maternal ccfDNA and organ-specific allogenous DNA in background of autogenous wild type ccfDNA, respectively).

Apart from well-established biological targets such as CTCs and ccfDNA, further target analytes can be analysed in context of liquid biopsy, such as extracellular vesicles (EVs) including their mRNA and miRNA content, circulating non-coding RNAs (miRNAs and others), and thrombocytes (platelets) (see Anfossi et al., Nat Rev Clin Oncol, 2018 September, Vol. 15(9):541-563; In't Veld, Wurdinger, Blood, 2019 Mar. 4, pii: blood-2018-12-852830).

Furthermore, genomic and epigenomic profiling of cell subpopulations comprised in the cell-containing bodily fluid sample, such as peripheral mononuclear blood cells (PMBCs), can be an useful biomarker for early diagnosis and monitoring of immunosurveillance in cancer patients (see Shen et al., Nature, 2018 November, Vol. 563(7732):579-583; Abu Ali Ibn Sina et al., Nature Communications, 2018, Vol. 9, Article number: 4915 and Nichita et al., Aliment Pharmacol Ther, 2014 March, Vol. 39(5):507-17).

Despite the well-recognized clinical potential of these biological targets that are comprised in bodily fluid samples such as blood, their utilization remains challenging. Existing methods that are based on the analysis of molecular biomarkers comprised in free circulating nucleic acids, EVs or CTCs for obtaining cancer-related information often have drawbacks with respect to sensitivity and/or robustness. Given the role of liquid biopsy as companion diagnostics in personalized medicine, complete and standardized workflows for liquid biopsy analyses are needed. Preanalytical conditions can significantly influence results of analytical tests. All liquid biopsy analytes require stabilization if tests are performed >3-4 hours after blood draw. Stabilization of the biological targets of interest must be sufficient and reliable. Currently there are blood stabilization tubes (BCT) available for either CTC analysis (e.g. CellSave, Transfix) or ctDNA analysis (Streck BCT, PAXgene Blood ccfDNA tube). Some of the tubes, such as Streck BCT, claim compatibility with CTC analysis, however such claims are basically limited to one particular CTC enrichment and detection technology. Moreover, the use of formaldehyde or formaldehyde-releasing substances (e.g. utilized in Streck BCT) has drawbacks, as they compromise the efficacy of extracellular nucleic acid isolation and efficacy of downstream analyses by induction of crosslinks between nucleic acid molecules or between proteins and nucleic acids.

It is an object of the present invention to overcome at least one drawback of the prior art and to provide improved liquid biopsy based analysis methods. In particular, it is an object of the present disclosure to provide methods that allow the reliable enrichment and analysis of multiple biological targets from a single cell-containing bodily fluid sample.

### SUMMARY

The present disclosure provides methods and thus workflows for the simultaneous stabilization, enrichment, and detection of a cell subpopulation such as e.g. rare target cells (e.g. CTCs) and extracellular nucleic acids such as extracellular DNA from the same cell-containing body fluid sample, as well as for the simultaneous stabilization, enrichment and analysis of other biological targets such as extracellular vesicles (EVs) from such stabilized sample. In addition, high quality intracellular nucleic acids such as genomic DNA (gDNA) can be isolated from the cellular fraction of the stabilized cell-containing bodily fluid sample. In particular, workflows are provided for the parallel liquid biopsy analyses of extracellular DNA, CTCs, EVs and gDNA from a single cell-containing body fluid sample that was collected and stabilized with the stabilizing technology according to the present disclosure.

According to a first aspect, a method is provided for stabilizing and enriching multiple biological targets comprised in a cell-containing bodily fluid, said method comprising
(A) contacting a cell-containing bodily fluid with a stabilizing composition comprising one or more of the following stabilizing agents:
   (a) at least one primary, secondary or tertiary amide,
   (b) at least one poly(oxyethylene) polymer, and/or
   (c) at least one apoptosis inhibitor,
   thereby providing a stabilized cell-containing bodily fluid sample;
(B) keeping the stabilized cell-containing bodily fluid sample for a stabilization period;
(C) processing the stabilized cell-containing bodily fluid sample in order to isolate three or more biological targets selected from the group consisting of rare cells, extracellular nucleic acids, extracellular vesicles and intracellular nucleic acids from the stabilized cell-containing bodily fluid.

The method may further comprise
(D) analysing the enriched three or more biological targets.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1: Immunocytochemical staining of MCF7 breast cancer cell line cells for human pan-Cytokeratin (green) and nuclei (blue). Upper panel demonstrated staining on untreated MCF7 cells. Lower panel represents staining on MCF7 cells stabilized in the stabilization solution of the present disclosure for 30 minutes.

FIG. 2: Blood samples after centrifugation with Ficoll-Paque density gradient medium. Blood samples collected into EDTA-containing BCT and diluted with PBS are taken as reference. The layers (from top to bottom) are: thrombocyte-rich plasma, PBMC ring, ficoll, red blood cell-enriched fraction. In the stabilized samples diluted with PBS the above mentioned fractions cannot be observed, and are present only after adding of 5% glucose or 0.9% NaCl+0.1M glycerol containing solution. This allows to restore the correct layer formation for obtaining the different fractions.

FIG. 3. Detection of spiked tumor cells from blood collected and stored in PAXgene Blood ccfDNA Tubes by AdnaTest ProstateCancerPanel AR-V7 at experimental time points 3, 24, 30, and 48 hrs after spike. Material: Blood collected into PAXgene Blood ccfDNA Tube, spiked with 20 LNCaP95 cells/5 ml blood or 20 μl PBS/5 ml blood and stored at 2-8° C. CTC enrichment and detection: AdnaTest ProstateCancerPanel AR-V7. FIG. 3A shows the results for samples spiked with 20 LNCaP95 cells/5 ml blood, and FIG. 3B shows the results for samples spiked with PBS only (no-spike control samples).

FIG. 4. Detection of spiked tumor cells in blood collected and stored in PAXgene Blood ccfDNA Tubes (FIG. 4A; n=11) and Streck Cell-Free DNA BCTs (FIG. 4B; n=8) by AdnaTest ProstateCancerPanel AR-V7 at experimental time points 3, 24, 48, and 72 hrs after spike. Material: Blood collected into PAXgene Blood ccfDNA Tubes and Cell-Free DNA BCTs (Streck) and spiked with 20 LNCaP95 cells/5 ml blood and stored at 2-8° C. (PAX) and RT (Streck). CTC enrichment and detection: AdnaTest ProstateCancer Panel AR-V7. FIGS. 4C and 4D show the performance of AdnaTest ProstateCancer Panel AR-V7 on PAXgene Blood ccfDNA stabilized blood spiked with 20 LNCaP95/5 ml blood stored at either 2-8° C. or room temperature for 3, 24, 48 or 72 hours. Material: Blood collected into PAXgene Blood ccfDNA Tubes, spiked with 20 LNCaP95 cells/5 ml blood. CTC enrichment and detection: AdnaTest ProstateCancerPanel AR-V7.

FIG. 5: Shows the cell capture efficiency using the Parsortix cell enrichment workflow when processing EDTA-stabilized blood or blood that was stabilized using the stabilizing technology according to the present disclosure. Blood collected in a PAXgene Blood ccfDNA tube is compatible with and can be processed even after three days of storage at room temperature.

FIG. 6: Analysis by RT-qPCR of RNA obtained from the purified EVs. Lower Ct values demonstrate better results.

FIG. 7: Schematic representation of the AdnaTestSelect and —Detect procedures with an option for collection of the CTC depleted blood after CTC enrichment for subsequent ccfDNA and gDNA isolation (see also FIG. 11).

FIG. 8: Evaluation of absolute differences in expression of 66 and 500 bp fragments of the human 18S rDNA gene (left and right panels, respectively) in samples after CTC enrichment (CTC-depleted blood) and control samples (i.e., samples without CTC enrichment) over the storage time. Box plots demonstrate median (horizontal line) and 25-75% interquartile range (box) and minimum and maximum of the data range (whiskers) and outliers (dots out of the whiskers). P values correspond to unpaired two-tailed t-test.

FIG. 9: Evaluation of gDNA yield from 200 μl of the cellular fraction from whole blood (i.e., samples without CTC enrichment, n=3 donors) and samples after CTC enrichment (n=8 donors) 3 h after spiking and at all time points (3-72 h, n=11 donors, 12 samples without CTC enrichment and 32 CTC-depleted samples). All data is shown as box plots, with median and quartiles within the box and 10/90 percentile as tails. Individual data points are overlaid as circles. P-values correspond to an unpaired two-tailed t-test.

FIG. 10: Overview over different options for liquid biopsy-based analyses compatible with PAXgene Blood ccfDNA tubes according to the method of the present invention.

FIG. 11: Exemplary liquid-biopsy based workflow for analyzing multiple targets from a single stabilized blood sample. As disclosed herein, using the stabilization technology according to the present invention also allows storage of the stabilized blood samples at room temperature for extended periods, prior to processing the stabilized blood sample according to step (D).

FIG. 12: Left panel: Blood samples collected into EDTA and PAXgene Blood ccfDNA Tubes (left and right, respectively) after centrifugation with Ficoll-Paque. The layers (from top to bottom) are: trombocyte-rich plasma, PBMC ring, red blood cell-enriched fraction. In the PAX samples diluted with PBS the above mentioned fractions are not clearly separated. Right panel: Relative differences in MNC recovery observed in PAX-stabilized samples in comparison to EDTA samples (taken as reference, n=8).

FIG. 13: Detection of spiked tumor cells from blood collected and stored in PAXgene Blood ccfDNA Tubes by AdnaTest ProstateCancerPanel AR-V7 at experimental time points 3, 24, 30, 48, 72, 120 and 144 hrs after spiking. Material: Blood collected into PAXgene Blood ccfDNA Tube, spiked with 20 LNCaP95 cells/5 ml blood and stored at 2-8° C. CTC enrichment and detection: AdnaTest ProstateCancerPanel AR-V7. FIG. 13 shows the performance of the AdnaTest ProstateCancer Panel AR-V7 test for detection of spiked tumor cells into blood collected into PAXgene Blood ccfDNA Tubes.

FIG. 14: Test performance in regard to storage temperature. CTC enrichment and detection by AdnaTest ProstateCancerPanel AR-V7 at experimental time points 3, 24, 48 and 72 hrs after spiking stored at room temperature (FIG. 14A) or at 3, 24, 30, 48, 72, 120 and 144 hrs after spiking stored at 2-8° C. (FIG. 14B). Material: Blood collected into PAXgene Blood ccfDNA Tubes and spiked with 20 LNCaP95 cells/5 ml blood.

FIG. 15: Test performance in regard to the number of spiked tumor cells for evaluating the limit of detection (LOD). Detection of spiked tumor cells from blood collected and stored in PAXgene Blood ccfDNA Tubes by AdnaTest ProstateCancerPanel AR-V7 at experimental time points 3, 24, 48, 72, 120 and 144 hrs after spiking. Material: Blood collected into PAXgene Blood ccfDNA Tube, spiked with either 5 LNCaP95 cells/5 ml blood (FIG. 15A) or 20 LNCaP95 cells/5 ml blood (FIG. 15B) and stored at 2-8° C. CTC enrichment and detection: AdnaTest ProstateCancerPanel AR-V7.

FIG. 16: Performance of the test in dependence on plasma generation regimen. Blood samples were used for CTC enrichment and the CTC-depleted blood was used for plasma generation (FIG. 16A). Alternatively, plasma was generated first (at 1900 g for 15 min) and the cellular fraction was then reconstituted with PBS up to the initial volume and used for CTC enrichment (FIG. 16B). Both methods of plasma generation performed similar well by enabling detection of spiked tumor cells from blood collected and stored in PAXgene Blood ccfDNA Tubes by AdnaTest ProstateCancerPanel AR-V7 at experimental time points 3, 24, 48, and 72 hrs after spiking.

FIG. 17: Performance of the test on EZ1 instrument in dependence on plasma generation regimen. The same two plasma generation methods as in FIG. 16 were performed by first CTC enrichment and then generating plasma (FIG. 17A) or first generating plasma and then enriching CTCs (FIG. 17B). Similar well results were observed when the same experiment as performed in FIG. 16 was conducted on EZ1 instrument (automated solution) using an AdnaTest adapted for EZ1. Spiked tumor cells from blood collected and stored in PAXgene Blood ccfDNA Tubes were detected by AdnaTest ProstateCancerPanel AR-V7 at experimental time points 3, 24, 48, 72 and 144 h hrs after spiking.

FIG. 18: Detection of spiked tumor cells from blood collected and stored in PAXgene Blood ccfDNA Tubes by AdnaTest ProstateCancerPanel AR-V7 (FIGS. 18A, 18C, 18E and 18G) compared to the AdnaTest Prostate Cancer (also referred to as “ProstateDirect”; FIGS. 18B, 18D, 18F and 18H) at experimental time points 3, 24, 48 and 72 hrs after spiking. Material: Blood collected into PAXgene Blood ccfDNA Tube, spiked with LNCaP95 cells/5 ml blood. FIGS. 18A and 18B show the performance of the tests by spiking 20 LNCaP95 cells/5 ml blood and storing at 2-8° C. FIGS. 18C and 18D show the performance of the tests by spiking 20 LNCaP95 cells/5 ml blood and storing at room temperature. FIGS. 18E and 18F show the performance of the tests by spiking 5 LNCaP95 cells/5 ml blood and storing at 2-8° C. FIGS. 18G and 18H show the performance of the test using the alternative plasma generation technique, wherein plasma was generated first and the cellular fraction was used for CTC enrichment.

FIG. 19: Performance of the AdnaTest ColonCancer. Detection of spiked tumor cells in blood collected and stored in PAXgene Blood ccfDNA Tubes (FIG. 19A) and ACD-A BCTs (FIG. 19B) by AdnaTest ColonCancer at experimental time points 3, 24, 48, and 72 hrs after spiking. Material: Blood collected into PAXgene Blood ccfDNA Tubes and ACD-A BCTs and spiked with 20 T48 cells/5 ml blood and stored at 2-8° C. CTC enrichment and detection: AdnaTest ColonCancer. FIGS. 19A and 19B show that the PAXgene Blood ccfDNA Tubes are compatible with the AdnaTest ColonCancer and allow for detection of tumor cells upon storage of samples within 72 h (100% sensitivity).

FIG. 20: Detection rates of spiked tumor cells (50 MCF7) after harvesting (FIG. 20A) and in-cassette staining (FIG. 20B). “T” refers to the number of days storage at room temperature (0, 1, 2, 3 days).

FIG. 21: IF staining of tumor cells after Parsortix enrichment. Fluorescent green—anti pan-kerating antibody staining (specific for tumor cells); fluorescent blue—DAPI (nuclear stain). Storage for 0 (T0) or 2 (T2) days.

FIG. 22: Detection of spiked tumor cells after Parsotix-based enrichment stored in PAXgene Blood ccfDNA Tubes by using the detection part of the AdnaTest ProstateCancerPanel AR-V7 (FIG. 22A) compared to the AdnaTest ProstateCancer (also referred to as “ProstateDirect”; FIG. 22B). FIG. 22 shows that cells spiked into PAX ccfDNA-collected blood samples and stored up to 3 days could be detected as efficiently as if spiked into EDTA-collected samples.

FIG. 22: Detection of spiked tumor cells after Parsotix-based enrichment stored in PAXgene Blood ccfDNA Tubes by using the detection part of the AdnaTest ProstateCancerPanel AR-V7 (FIG. 22A) compared to the AdnaTest ProstateCancer (also referred to as “ProstateDirect”; FIG. 22B). FIG. 22 shows that cells spiked into PAX ccfDNA-collected blood samples and stored up to 3 days could be detected as efficiently as if spiked into EDTA-collected samples.

FIG. 23: Multimodal workflow for the analysis of ccfRNA, ccfDNA and gDNA as used in Example 7.

FIG. 24a: CT values of qPCR analysis of miR150, let7a and miR451 micro RNAs, ACTB mRNA and 18S rDNA (ccfDNA) in PAXgene and EDTA plasma, generated directly after blood collection (test time point=TTP0) and extracted using the indicated kit.

FIG. 24b: Calculated fold change (relative to TTP0) of qPCR analysis of miR150, let7a, miR451 micro RNAs and ACTB mRNA of PAXgene and EDTA plasma, generated after 1, 3 or 6 days of whole blood storage. RNA was extracted using the indicated kit.

FIG. 25a: CT values of qPCR analysis of miR150, let7a and miR451 micro RNAs in PAXgene, Streck cfDNA, Streck RNA and Biomatrica plasma, generated directly after blood collection (TTP0) and extracted using the indicated kit.

FIG. 25b: Calculated fold change (relative to TTP0) of qPCR analysis of miR150, let7a, miR451 micro RNAs and 18S rDNA in PAXgene, Streck cfDNA, Streck RNA and Biomatrica plasma, generated after 3 days of storage (T3d). RNA was extracted using the indicated kit.

FIG. 26: Concentration and DNA integrity index (DIN) assessment of gDNA extracted from whole blood in PAXgene Blood ccfDNA Tubes, Streck cfDNA, Streck RNA and Biomatrica tubes. DNA was extracted from cellular fraction after first plasma centrifugation step using the QIAamp Blood DNA Kit and analyzed with the Agilent Genomic DNA ScreenTape® on the TapeStation System.

### DETAILED DESCRIPTION

The present disclosure provides an advantageous method for stabilizing and enriching multiple biological targets comprised in a cell-containing bodily fluid, said method comprising
(A) contacting a cell-containing bodily fluid with a stabilizing composition comprising one or more of the following stabilizing agents:
   (a) at least one primary, secondary or tertiary amide,
   (b) at least one poly(oxyethylene) polymer, and/or
   (c) at least one apoptosis inhibitor,
   thereby providing a stabilized cell-containing bodily fluid sample;
(B) keeping the stabilized cell-containing bodily fluid sample for a stabilization period;
(C) processing the stabilized cell-containing bodily fluid sample in order to enrich three or more biological targets selected from the group consisting of at least one cell subpopulation, extracellular nucleic acids, extracellular vesicles and intracellular nucleic acids from the stabilized cell-containing bodily fluid.

The method may further comprise
(D) further processing the enriched three or more biological targets for analysis.

Each individual step of the method as well as suitable and preferred embodiments of the present method are subsequently described in detail.

### Step (A)

In step (A), a cell-containing bodily fluid is contacted with a stabilizing composition which comprises one or more, two or more, or all three of the following stabilizing agents:
(a) at least one primary, secondary or tertiary amide,
(b) at least one poly(oxyethylene) polymer, and/or
(c) at least one apoptosis inhibitor,

whereby a stabilized cell-containing bodily fluid sample is provided. The stabilizing composition may be comprised, e.g. pre-filled—in a collection vessel, e.g. a collection tube. The cell-containing biological sample may be introduced into the collection vessel. The step of contacting the cell-containing biological body fluid with the stabilizing composition occurs ex vivo.

Advantageous stabilizing effects of the individual agents in stabilizing cell-containing body fluid samples and advantageous stabilizing compositions comprising combinations of these agents are disclosed e.g. in WO2013/045457, WO2013/045458, WO2014/146780, WO2014/146781, WO2014/146782, WO2014/049022, WO2015/140218 and WO2017/085321, herein incorporated by reference. Advantageous stabilizing compositions comprising combinations of the stabilizing agents (a) to (c) are also described elsewhere herein and it is referred to this disclosure.

As is demonstrated in the subsequent examples and supported by the aforementioned documents, the parallel processing and analysis of different biological targets of interest comprised in the cell-containing bodily fluid is possible. The stabilization technology that is used in the present method advantageously stabilizes numerous biological targets of interest, including rare cells (such as e.g. circulating tumor cells), extracellular nucleic acids (such as e.g. extracellular DNA and RNA), extracellular vesicles and intracellular nucleic acids (such as genomic DNA) upon contact with the cell-containing bodily fluid. As is demonstrated in the subsequent examples, multiple biological targets of interest can be recovered from the stabilized cell-containing bodily fluid sample and subjected to classic analysis and detection methods. This enables the multimodal analyses of different biological targets of high interest from a single stabilized cell-containing bodily fluid.

### Step (B)

In (B) the stabilized cell-containing bodily fluid sample is kept for the intended stabilization period.

The stabilized cell-containing bodily fluid sample may e.g. be processed directly or shortly after stabilization (e.g. within 3 hours) or may be kept for a prolonged storage period. It is a particular advantage that the stabilized cell-containing body fluid samples may be kept for prolonged storage periods. The biological targets comprised in the stabilized sample are preserved also over prolonged storage periods.

In embodiments, (B) comprises storing the stabilized cell-containing bodily fluid sample prior to processing step (C). Storing may comprise e.g. transferring the stabilized cell-containing bodily fluid sample from the site of collection and stabilization to a distinct site for further processing.

The stabilized cell-containing bodily fluid sample may be kept for up to 12 h or up to 24 h prior to performing processing step (C). As is demonstrated in the examples, the stabilized cell-containing bodily fluid sample may be kept for up to 30 h, up to 36 h or up to 48 h prior to performing processing step (C). In embodiments, the stabilized cell-containing bodily fluid sample is kept for up to 50 h or up to 72 h prior to performing processing step (C).

When keeping the stabilized cell-containing bodily fluid sample for the intended stabilization period, it is advantageous if the stabilized sample is not subjected to a freezing step. A freezing step may damage comprised cells. Avoiding a freezing step is thus advantageous because it supports the preservation of the cell-containing bodily fluid sample.

In embodiments, the stabilized cell-containing bodily fluid sample is kept at room temperature (e.g. 15-25° C.) during the intended stabilization period. In other embodiments, the sample is cooled and is e.g. kept e.g. at a temperature of 1-14° C., such as 1-12° C. or 2-10° C. or 2-8° C. In embodiments the stabilized cell-containing bodily fluid sample such as blood may be kept for up to 72 hours at 2-8° C.

In embodiments, the stabilized cell-containing body fluid sample is kept for at least 4 h or at least 6 h prior to performing processing step (C). In embodiments, the stabilized cell-containing body fluid sample is kept for at least 8 h or at least 12 h prior to performing processing step (C). In embodiments, the stabilized cell-containing body fluid sample is kept for at least 24 h, at least 30 h or at least 48 h up to 72 h (or longer) prior to performing processing step (C).

### Step (C)

After the stabilization period, the stabilized cell-containing bodily fluid sample is processed in order to enrich three or more biological targets selected from the group consisting of at least one cell subpopulation, extracellular nucleic acids, extracellular vesicles and intracellular nucleic acids from the stabilized cell-containing bodily fluid.

As disclosed herein, it is highly advantageous that multiple different biological targets of interest are stabilized within the cell-containing bodily fluid and can subsequently be recovered from the same stabilized sample, and this even after prolonged stabilization periods. This enables the parallel/simultaneous recovery and analysis of multiple different biological targets obtained from a single stabilized cell containing bodily fluid in efficient workflows.

As disclosed herein, in one embodiment, the at least one cell population that is enriched comprises or essentially consists of target rare cells. In embodiments, the target rare cells are tumor cells, such as circulating tumor cells (CTCs). As discussed in the background, tumor cells, such as CTCs, represent a biological target of particular interest.

As is described herein, it is advantageous to separate the stabilized cell-containing bodily fluid sample into at least one cell-depleted fraction and at least one cell-containing fraction. The cells comprised in the cell-containing bodily sample can thereby be concentrated in the provided cell-containing fraction. The at least one cell-containing fraction may comprise nucleated cells. In embodiments, the at least one cell-containing fraction essentially consists of nucleated cells.

Suitable and preferred embodiments for processing the stabilized cell-containing bodily fluid in step (C) as well as the biological targets are described in the following.

### Embodiment A

According to embodiment A, processing in (C) comprises
(aa) separating the stabilized cell-containing bodily fluid sample into at least one cell-containing fraction and at least one cell-depleted fraction;
(bb) further processing the cell-containing fraction, wherein further processing the cell-containing fraction comprises
   (i) enriching at least one cell subpopulation, e.g. comprising target rare cells, from the cell-containing fraction; and/or
   (ii) enriching, e.g. purifying, intracellular nucleic acids (e.g. genomic DNA) from the cell-containing fraction;
(cc) further processing the cell-depleted fraction, wherein further processing the cell-depleted fraction comprises
   (i) enriching, e.g. purifying, extracellular nucleic acids (e.g. extracellular DNA), from the cell-depleted fraction; and/or
   (ii) enriching extracellular vesicles from the cell-depleted fraction.

In this embodiment, the stabilized cell-containing bodily fluid sample (e.g. blood) is separated in (aa) into at least one cell-containing fraction (e.g. comprising nucleated blood cells and CTCs) and a cell-depleted fraction (e.g. blood plasma). Suitable separation methods are known in the art (e.g. involving centrifugation and/or filtration) and described elsewhere herein. E.g. when processing a stabilized blood sample (anticoagulated) according to step (aa) using a centrifugation based separation method, the stabilized blood sample may be separated into a cell-depleted fraction (plasma), a cell-containing fraction (buffy coat, comprising leukocytes and, if present, CTCs and optionally platelets) and an erythrocytes fraction. The buffy coat may be further processed as cell-containing fraction in step (bb) and the plasma fraction may be further processed as cell-depleted fraction in (cc).

The obtained cell-containing fraction of interest is then further processed in (bb). At least one cell subpopulation such as target rare cells (e.g. CTCs) may be enriched from the obtained cell-containing fraction (see (i)). Furthermore, intracellular nucleic acids (e.g. genomic DNA) may be enriched and thus purified from the cell-containing fraction (see (ii)). In embodiments, at least one cell subpopulation, e.g. comprising rare cells (e.g. CTCs), and intracellular nucleic acids (e.g. genomic DNA) are both enriched as biological targets of interest from the cell-containing fraction. E.g. one may first isolate the cell subpopulation of interest, e.g. comprising rare cells (e.g. CTCs), from the cell-containing fraction in (i), before subsequently purifying in (ii) intracellular nucleic acids (e.g. genomic DNA) from the remaining cell-containing fraction from which the target cell subpopulation (e.g. rare cells) were removed/depleted. Advantageously, this embodiment allows to use the full volume of the cell-containing fraction for the isolation of the target cell subpopulation, which comprises in one embodiment rare cells (such as CTCs). This is advantageous considering that specific cells such as CTCs are often so rare that it is desirous to process a large volume of the cell-containing fraction in order to ensure that rare cells (such as CTCs) if comprised can be enriched in sufficient amounts for subsequent detection. In other embodiments, the cell-containing fraction is divided into at least two aliquots, wherein at least one aliquot is used for enriching the cell subpopulation of interest (e.g. comprising rare cells) and at least one aliquot is used for enriching intracellular nucleic acids, such as genomic DNA.

The obtained cell-depleted fraction is further processed in (cc) in order to isolate extracellular nucleic acids and/or enrich extracellular vesicles from the cell-depleted fraction (e.g. plasma). As disclosed herein, in advantageous embodiments, extracellular DNA is purified from the cell-depleted fraction (e.g. plasma). Furthermore, as demonstrated in the examples, extracellular vesicles may be enriched from the cell-depleted fraction. Exemplary suitable and preferred methods for enriching extracellular vesicles are also described below. In embodiments, extracellular vesicles and extracellular nucleic acids, preferably extracellular DNA, are both enriched from the cell-depleted fraction. E.g. one may first isolate extracellular vesicles from the cell-depleted fraction, before enriching extracellular DNA from the remaining cell-depleted fraction from which the extracellular vesicles were removed in advance. In further embodiments, the cell-depleted fraction is divided into at least two aliquots, wherein at least one aliquot is used for enriching extracellular vesicles and at least one aliquot is used for purifying extracellular nucleic acids, such as extracellular DNA, therefrom.

### Embodiment B

According to embodiment B, processing in (C) comprises
(aa) enriching at least one cell subpopulation, e.g. comprising target rare cells, from the stabilized cell-containing bodily fluid sample;
(bb) separating the stabilized cell-containing bodily fluid sample from which the cell subpopulation (e.g. comprising target rare cells) was enriched and thus removed into a cell-containing fraction and a cell-depleted fraction;
(cc) further processing the cell-depleted fraction, wherein further processing the cell-depleted fraction comprises
   (i) enriching extracellular nucleic acids, optionally extracellular DNA, from the cell-depleted fraction; and/or
   (ii) enriching extracellular vesicles from the cell-depleted fraction; and
(dd) optionally enriching intracellular nucleic acids, preferably genomic DNA, from the cell-containing fraction.

In step (aa) at least one cell subpopulation, e.g. comprising target rare cells (e.g. CTCs), is enriched from the stabilized cell-containing bodily fluid sample. To first isolate a target cell subpopulation of interest, e.g. comprising rare cells, from the biological sample before separating the stabilized sample into a cell-containing and a cell-depleted fraction reduces the overall handling time of the cell subpopulation. This is particularly advantageous in case the cell subpopulation comprises or essentially consists of rare cells which to prevent damage to these rare and thus precious cells. In one embodiment, rare cells (such as CTCs) are enriched from the entire stabilized cell-containing bodily fluid sample. Advantageously, this allows to use the full collected sample volume for the isolation of rare cells (such as CTCs). This is advantageous considering that specific cells such as CTCs are often so rare that it is desirous to process larger sample volumes in order to ensure that comprised rare cells (such as CTCs) can be enriched and detected.

In step (bb) the stabilized cell-containing bodily fluid sample from which target rare cells (or other cell subpopulation of interest) were removed is separated into a cell-containing fraction and a cell-depleted fraction. Accordingly, in case the full collected volume of the stabilized cell-containing bodily fluid sample was used for enriching rare cells in step (aa), the whole stabilized cell-containing bodily fluid sample from which rare cells were removed, or if desired a portion thereof, is processed to provide the cell-containing fraction and the cell-depleted fraction.

In step (cc), the cell-depleted fraction is further processed. Details were described in conjunction with embodiment A above and it is referred to the respective disclosure which also applies here.

Furthermore, intracellular nucleic acids such as genomic DNA may be enriched from the cell-containing fraction in step (dd).

### Embodiment C

According to embodiment C, processing in (C) comprises
(aa) dividing the stabilized cell-containing bodily fluid sample into at least two aliquots and enriching at least one cell population of interest, e.g. comprising rare cells, from at least one of the provided aliquots;
(bb) providing at least one cell-containing fraction and at least one cell-depleted fraction;
(cc) further processing the cell-depleted fraction, wherein further processing the cell-depleted fraction comprises
   (i) enriching extracellular nucleic acids, optionally extracellular DNA, from the cell-depleted fraction; and/or
   (ii) enriching extracellular vesicles from the cell-depleted fraction; and
(dd) optionally enriching intracellular nucleic acids, preferably genomic DNA, from the cell-containing fraction.

In step (aa), the stabilized cell-containing bodily fluid sample is divided into at least two aliquots. At least one aliquot is used for enriching at least one cell population of interest, which may e.g. comprise or essentially consist of the target rare cells (e.g. CTCs). Thereby, at least one aliquot of the stabilized cell-containing bodily fluid sample is provided from which the rare cells were removed. The same applies if another cell subpopulation of interest is enriched. At least one further aliquot corresponds to the original stabilized cell-containing bodily fluid from which the rare cells (or other cell subpopulation of interest) were not removed.

In step (bb) at least one cell-containing fraction and at least one cell-depleted fraction is provided. Step (bb) may comprise separating the stabilized cell-containing bodily fluid sample from which the target cell population (e.g. comprising or essentially consisting of rare cells such as CTCs) was enriched and/or any remaining stabilized cell-containing bodily fluid sample (aliquot) that was not used for enriching the target cell population in step (aa) into a cell-containing fraction and a cell-depleted fraction. In case the stabilized cell-containing bodily fluid sample was divided into at least two aliquots, it is possible to process only the aliquot from which the target cells were not removed in order to provide the cell-containing fraction and the cell-depleted fraction. Alternatively, the at least one aliquot from which target cells were removed may be re-united with the further aliquot of the original stabilized bodily fluid sample from which the target cells were not removed. Such pooling advantageously increases the volume of the obtained cell-depleted and cell-containing fractions which is beneficial for the further processing and analysis of these fractions.

Step (cc) and optional step (dd) correspond to embodiment B and it is referred to the above disclosure.

Exemplary suitable and preferred methods for separating the sample into at least one cell-containing fraction and at least one cell-depleted fraction are also described below. Such methods can be used in step (C), in particular in embodiments A to C.

Exemplary suitable and preferred methods for enriching rare cells such as CTCs and other target cell subpopulations that can be used in step (C), in particular in embodiments A to C, are described below. As disclosed herein, the recovered target cells, such as rare cells, may be further processed in step (D), e.g. in order to isolate intracellular nucleic acids (e.g. RNA), followed by the subsequent detection.

Exemplary suitable and preferred methods for enriching extracellular vesicles such as exosomes that can be used in step (C), in particular in embodiments A to C, are also described below. As disclosed herein, the recovered extracellular vesicles may be further processed in step (D), e.g. in order to isolate nucleic acids (e.g. RNA), followed by the subsequent detection.

Methods for Separating a Cell-Containing Bodily Fluid Sample into a Cell-Containing Fraction and a Cell-Depleted Fraction

Methods for separating a cell-containing bodily fluid into a, i.e. at least one, cell-containing fraction and a, i.e. at least one, cell-depleted fraction are well-known in the art and therefore, do not need to be described in detail. Common methods include, but are not limited to, centrifugation, filtration and density gradient centrifugation. The different methods may also be combined. Such common methods may be advantageously used in conjunction with the stabilization technology according to the present disclosure, which advantageously allows to avoid the use of cross-linking agents for stabilization, so that common, established methods may be used. The methods are performed so that the integrity of the comprised cells is preserved. This is advantageous because cell breakage during separation would contaminate e.g. the extracellular nucleic acids that are comprised in the cell-depleted fraction with cellular nucleic acids that are released from disrupted cells.

According to one embodiment, at least one centrifugation step is performed, in order to separate a cell-containing fraction from a cell-depleted fraction. In embodiments, the centrifugation may be performed e.g. in the range of 800 to 3000×g, such as 1000 to 2500×g or 1500 to 2000×g. The centrifugation duration may be e.g. in the range of 5 to 20 min, such as 10-15 min. Suitable conditions can be chosen by the skilled person. The cell-depleted fraction can be recovered as supernatant. The cell-depleted fraction may be removed from the obtained cellular fraction(s) and subjected to a second centrifugation step, optionally performed at higher speed, in order to ensure that any remaining cells and particulate matter (e.g. cell debris) are removed from the cell-depleted fraction. This may be advantageous for the subsequent purification of extracellular nucleic acids, such as extracellular DNA, from the cell-depleted fraction. It is also within the scope of the present disclosure to perform a filtration step in order to provide the cell-depleted fraction. Such methods are well-known in the art and are e.g. used for obtaining blood plasma from blood samples for subsequent purification of extracellular nucleic acids such as extracellular DNA (see e.g. Chiu et al, 2001 Clinical Chemistry 47:9 1607-1613; Sorber et al, Cancers 2019, 11, 458). In case it is desired to recover exosomes and/or platelets as biological target(s) of interest from the cell-depleted fraction the separation protocol(s) are chosen such that the exosomes and/or platelets remain in the cell-depleted fraction and therefore are available for recovery therefrom. A cellular fraction, e.g. obtained after a first centrifugation step, may be used as cell-containing fraction and further processed as described herein (e.g. in order to isolate intracellular nucleic acids such as genomic DNA and/or enrich target cells (e.g. CTCs) therefrom).

Suitable centrifugation and/or filtration based separating methods may include but are not limited to:
Centrifugation at 1900×g (15 min) to separate a cell-depleted fraction from the cellular fraction(s) and centrifugation of the cell depleted fraction at 1900×g (10 min).
Centrifugation 1600×g (10 min) to separate a cell-depleted fraction from the cellular fraction(s) and centrifugation of the cell depleted fraction at 16000×g (10 min).
Centrifugation 1600×g (10 min) to separate a cell-depleted fraction from the cellular fraction(s) followed by filtration of the cell-depleted fraction, e.g. using a 0.2 μm-0.8 μm filter.
Centrifugation 1600×g (10 min) and 16000 g (10 min) followed by filtration, e.g. using a 0.2 μm-0.8 μm filter.
Centrifugation 1000 rpm (10 min) and 3000 rpm (10 min).

Further combinations and variations are also possible.

The provided cell-depleted fraction is in embodiments substantially cell-free in order to avoid contamination of e.g. comprised biological targets (e.g. extracellular nucleic acids or extracellular vesicles) with cell components. Such cell-free fraction may be obtained using the centrifugation and/or filtration based methods described above. The obtained cell-depleted/cell-free fraction may be transferred into a new vessel. It may be processed directly, e.g. in order to purify extracellular nucleic acids and/or extracellular vesicles therefrom, or may be stored (e.g. cooled or frozen) until use. The obtained cell-containing fraction that is further processed may comprise nucleated cells and target cells (such as e.g. rare cells) and/or intracellular nucleic acids (e.g. genomic DNA) may be isolated therefrom.

According to one core embodiment, the cell-containing bodily fluid is blood. Blood samples are of core interest, because blood samples are widely used for diagnostic purposes. In case the cell-containing bodily fluid is blood, it is preferred that the stabilizing composition comprises an anticoagulant, e.g. a chelating agent such as EDTA. The stabilized blood sample may be processed in order to provide a cell-depleted plasma fraction and a cell-containing cellular fraction, such as buffy coat, which is then further processed. Methods for generating plasma are well known in the art and include but are not limited to centrifugation and filtration and combinations of such methods.

### Subpopulations of Cells and Enrichment of Such Subpopulations, in Particular Rare Cells Such as Circulating Tumor Cells

According to one embodiment, step (C) comprises enriching a cell subpopulation from the stabilized cell-containing bodily fluid sample. The target cell subpopulation may be enriched directly from the stabilized cell-containing bodily fluid, or it may be enriched from a cell-containing and thus cellular fraction of the stabilized cell-containing bodily fluid (which may be obtained by separating the stabilized cell-containing bodily fluid sample into a cell-containing and a cell-depleted fraction). The enriched subpopulation of cells may be processed and analyzed further as described herein (e.g. by analyzing obtained cells and/or isolating intracellular nucleic acids therefrom).

The desired cell subpopulation may be enriched using methods known in the art. Suitable methods are disclosed below in conjunction with the enrichment of rare cells and similar methods may also be used for other cell populations. E.g. specific cells may be enriched based on their cell surface properties using affinity capture based methods. Furthermore, cells may be separated and thus enriched based on their density. E.g. density gradient centrifugation allows to enrich PBMCs and other cell types, in specific layers. Specific cells, respectively a cell population may also be enriched by sorting techniques, such as FACS sorting.

According to one embodiment, step (C) comprises enriching rare cells. Thus, according to one embodiment, the enriched cell subpopulation comprises target rare cells. The enriched cell subpopulation may also essentially consist of the target rare cells. This depends on the used enrichment method.

Rare cells are low-abundant cells in a larger population of background cells. Rare cells are found typically with a concentration of or below 1 in 10⁵ cells. Therefore, the detection, quantification and enrichment of rare cells are challenging. Rare cells are highly important for various applications such as the diagnosis and prognosis of many cancers, prenatal diagnosis, and the diagnosis of viral infections. Typical rare cells are circulating tumor cells (CTCs), circulating fetal cells (e.g. circulating in maternal blood), stem cells, and cells infected by virus or parasites. Such rare cells are e.g. found in blood samples and other bodily fluids and may be enriched therefrom. Further rare cells types that may be enriched are circulating endothelial cells (CECs) and circulating endothelial progenitor cells (EPCs). Circulating mature endothelial cells (CECs), which are potential biomarkers for endothelial dysfunction in cancer, diabetes, cardio-vascular or acute kidney diseases have been observed with a frequency of 10-100 CECs in 10⁶-10⁸ white blood cells. Compared to that, the estimated frequency of CTCs is even lower, ranging from 1 to 10 CTCs in 10⁶-10⁸ white blood cells.

Different methods are known and described in the art for the enrichment of rare cells such as CTCs and the known methods can be used in conjunction with the present invention (see e.g. Neumann et al., Comput Struct Biotechnol J, 2018, Vol. 16: 190-195; Haber et al, Cancer Discov. 2014 June; 4 (6): 650-661 and Chen, Lab Chip: 2014 Feb. 21; 14 (4): 625-645). Enrichment, separation or quantification of rare cells can be done by various methods, e.g. based on physical properties like cell size, density, deformability, shape, electrical polarizability and magnetic susceptibility and/or biological properties of the cells, such as surface properties (e.g. marker gene expression on the cell surface). Gradient-based centrifugation (e.g. using a Ficoll gradient) is one commonly used method to enrich for a specific cell type with a certain density. Filtration enables enrichment of rare cells based on cell size. Another CTC enrichment principle is using microfluidics. In comparison to filtration methods, microfluidic systems allow to harvest a CTC-enriched cell suspension for downstream analysis such as immunofluorescent labelling for single cell isolation. CTCs and also other rare cells can also be separated based on differences in their electrical charge. Overall, CTC enrichment strategies fall broadly within different classes, depending on whether they rely on physical properties of tumor cells, their expression of unique cell surface markers, or the depletion of abundant cells (e.g. normal leukocytes) to enrich untagged CTCs. For enrichment of CTCs, also immunomagnetic methods can be used, e.g. based on antibody-mediated capture of cancer cells.

According to one embodiment, the target rare cells are tumor cells that are comprised in the cell-containing bodily fluid sample. Preferably, circulating tumor cells (CTCs) are obtained as target rare cells from the stabilized bodily fluid sample, such as a stabilized blood sample. As disclosed in the background, circulating tumor cells are well known in the art. Commonly, CTCs are cells that have shed into the vasculature or lymphatic from a primary tumor and are carried around the body in the circulation. CTCs can be shed actively or inactively. They can circulate in the blood and lymphatic system as single cells or as aggregates, so called circulating tumor microemboli. CTCs thus originate from the primary tumor and can constitute living seeds for the subsequent growth of additional tumors (metastases) in vital distant organs. They are considered to be closely related to cancer metastasis which is the leading cause of cancer mortality. CTCs can also originate from metastases. CTCs have been identified in many different cancers and it is widely accepted that CTCs found in peripheral blood originate from solid tumors and are involved in the haematogenous metastatic spread of solid tumors to distant sites. The term CTCs as used herein in particular includes circulating cells derived from all types of tumors, especially of solid tumors, in particular of metastasizing solid tumors. The term CTC as used herein inter alia includes but is not limited to (i) CTCs that are confirmed cancer cells with an intact, viable nucleus that express cytokeratins or epithelial marker molecules like EpCam and have an absence of CD45; (ii) cytokeratin negative (CK−) CTCs that are cancer stem cells or cells undergoing epithelial-mesenchymal transition (EMT) which may lack expression of cytokeratins or epithelial markers like EpCam and CD45; (iii) apoptotic CTCs that are traditional CTCs that are undergoing apoptosis (cell death); (iv) small CTCs that usually are cytokeratin positive and CD45 negative, but with sizes and shapes similar to white blood cells, (v) dormant CTCs, as well as CTC clusters of two or more individual CTCs, e.g. of any of the aforementioned types of CTCs or a mixture of said types of CTCs are bound together. A CTC cluster may contain e.g. traditional, small and/or CK− CTCs.

CTCs are generally very rare cells within a bodily fluid. To provide information on CTCs, the enrichment of tumor cells or the removal of other nucleated cells in blood is required. Any method can be used in conjunction with the present method that is suitable to enrich CTCs from the stabilized cell-containing bodily fluid sample or the obtained cell-containing fraction thereof. Because CTCs are often rare, common CTC enrichment procedures mostly co-isolate other cell types together with the desired CTCs so that the enriched CTCs are comprised to a certain extent in the background of normal cells. Such methods nevertheless enrich CTCs and therefore are methods useful for enriching CTCs for analysis. Methods for enriching CTCs from various biological samples are well known in the art and were also summarized above. Exemplary suitable methods are briefly described in the following.

CTCs may be enriched using various physical and/or affinity capture based methods. CTCs may be enriched by methods that include a positive selection of CTC cells, e.g. by a method directly targeting CTCs, or methods that include a negative selection, e.g. by depleting non-CTC cells (e.g. leukocytes in case of blood). Also feasible are methods that enrich CTCs by size using e.g. filtration based methods, deformability or density or other physical methods. Moreover, a combination of the aforementioned methods can be used.

According to a preferred embodiment, CTCs are enriched by affinity capture. Such affinity based capture methods specifically bind CTCs to a surface (e.g. a bead, membrane or other surface). Specificity for CTCs is achieved by using one or more binding agents (e.g. antibodies) that bind to structures, e.g. epitopes or antigens, present on the CTCs. In embodiments, said one or more binding agents bind tumor-associated markers present on the CTCs. E.g. CTCs may be enriched using antibody-coated solid phase (e.g. magnetic beads) that can capture CTC cells. For CTC capture, a combination of two or more antibodies can be used that bind with high specificity and affinity to epitopes or antigens on the desired CTC cells. Binding agents may also be selected to target epitopes or antigens present on the CTCs depending on the tumor type. E.g. different structures, e.g. epitopes or antigens, may be present on the CTCs that can be targeted by the binding agent (e.g. antibody) depending on the primary tumor type, also taking potential EMT or tumor stemcell phenotype changes into consideration. The use of an according binding agent (e.g. antibody) based capturing platform is advantageous since it may also enrich CTCs which have undergone phenotype changes in the course of e.g. epithelial to mesenchymal transition (EMT) or display tumor-stemness. According to a preferred embodiment, the epitopes targeted by the binding agent are epithelial- and/or tumor-associated antigens, such as e.g. EpCAM, EGFR and HER2. A commercially available system for enriching circulating tumor cells is the AdnaTest (QIAGEN).

Another method that is based on positive selection and therefore represents a suitable CTC enrichment method for obtaining CTCs is based on the enumeration of epithelial cells that are separated from blood by antibody-magnetic nanoparticle conjugates that target epithelial cell surface markers, EpCAM, and the subsequent identification of the CTCs with fluorescently labeled antibodies against cytokeratin (CK 8, 18, 19) and a fluorescent nuclear stain. An according method is used in the commercially available system of CellSearch (Menarini/Veridex LLC). Other known methods for CTC enrichment and thus CTC isolation include but are not limited to Epic sciences method, the ISET Test, the use of a Microfluidic cell sorter (μFCS which employs a modified weir-style physical barrier to separate and capture CTCs e.g. from unprocessed whole blood based on their size difference), ScreenCell (a filtration based device that allows sensitive and specific isolation of CTCs e.g. from human whole blood), Clearbridge, Parsortix and IsoFlux.

According to one embodiment, the stabilized sample is a blood sample and step (C) comprises enriching PBMCs from the stabilized sample, optionally using a density gradient centrifugation based enrichment method. Suitable methods are described below. As disclosed in the background, the genomic and/or epigenomic profiling of peripheral mononuclear blood cells (PMBCs) represents a biomarker of interest for early diagnosis and monitoring of immunosurveillance in cancer patients. Furthermore, it may be used for the analysis of comprised CTCs, e.g. by isolating intracellular nucleic acids such as RNA and detecting CTC specific target nucleic acid molecules. Furthermore, the enriched PBMC fraction may be used for further enriching and thus purifying specific cell types therefrom, such as CTCs.

According to one embodiment, the cell-containing bodily fluid sample is blood and step (C) comprises enriching target lymphocytes as cell subpopulation from the stabilized sample. According to one embodiment, the lymphocytes are selected from T4 and/or T8 lymphocytes. According to one embodiment, the stabilized blood sample was obtained from a patient with immune deficiency. Analysis of T4 and T8 lymphocytes in such samples is of particular diagnostic value.

According to one embodiment, the cell-containing bodily fluid sample is blood and step (C) comprises enriching platelets as cell subpopulation from the stabilized sample, optionally wherein step (D) is performed and comprises isolating RNA from the enriched platelets. Methods for enriching platelets from a blood sample are known in the art and may be used in conjunction with the present invention. In embodiments, a platelet-rich plasma (PRP) is obtained from the stabilized (anticoagulated) blood sample by centrifugation. Suitable methods for obtaining platelet-rich plasma are described in the art (see also Sorber et al, 2019) and can be used and/or adapted to the present disclosure. The platelet-rich plasma is depleted from other white and red blood cells. The platelets may then be isolated from the obtained platelet-rich plasma, respectively a portion thereof, using methods known in the art. In embodiments, the remaining plasma portion that was not used for isolating the platelets may be further processed for isolating extracellular nucleic acids (e.g. ccfDNA) and/or exosomes therefrom. In embodiments, the remaining plasma portion is again centrifuged and/or filtrated in order to remove remaining cells or cell debris, prior to isolating extracellular nucleic acids and/or exosomes from the obtained supernatant.

According to one embodiment, the cell-containing bodily fluid sample is blood and step (C) comprises enriching blast cells as a target cell subpopulation from the stabilized sample. The blast cells are enriched by affinity capture, optionally using magnetic particles. Blast cells may be e.g. enriched by targeting cell surface markers, optionally CD34 and/or CD117. Analysis of blast cells is e.g. useful where the stabilized blood sample was obtained from a patient with acute myeloid leukemia.

As noted above, further rare cells types that may be enriched from the stabilized cell-containing bodily fluid sample are circulating endothelial cells (CECs) and circulating endothelial progenitor cells (EPCs). Such target cells may be identified and enriched on the basis of specific markers, including but not limited to CD31, CD34, CD105, CD133 and CD146.

### Density Gradient Centrifugation Step

According to one embodiment, processing step (C) comprises subjecting the stabilized blood sample or a cellular fraction thereof to a density gradient centrifugation step. Performing a density gradient centrifugation step allows to separate the stabilized cell-containing bodily fluid sample into a cell-depleted plasma fraction (or cell-depleted liquid in case of processing a cellular fraction that was obtained from the stabilized cell-containing bodily fluid sample as input material) and different cell-containing fractions. In embodiments, the stabilized cell-containing bodily fluid sample is first processed in step (C), in order to obtain a cell-containing fraction and a cell-depleted fraction. Methods as described above (e.g. centrifugation and/or filtration) may be used for this purpose. E.g. a stabilized blood sample may be separated into a plasma fraction and a cellular fraction. The obtained plasma fraction may then be used for the enrichment of (i) extracellular nucleic acids and/or (ii) extracellular vesicles, as described elsewhere. The obtained cellular fraction may then be subjected to density gradient centrifugation. For this purpose, the cellular fraction may be diluted using a dilution solution. The diluted cellular fraction is then subjected to density gradient centrifugation. The density gradient centrifugation procedure may then be performed as it is known and described for the cell-containing bodily fluid, such as e.g. blood.

Embodiments of density gradient centrifugation are described in the following, by way of example with a stabilized blood sample. However, also other types of stabilized cell-containing bodily fluid samples may be processed accordingly.

The stabilized blood sample (or the cellular fraction thereof) is contacted with a density gradient medium. Suitable density gradient mediums are commercially available and include but are not limited to Ficoll®, Ficoll®-Paque and Lymphopure. Density gradient centrifugation techniques (such as Ficoll® Paque, OncoQuick®) can be used to separate peripheral blood mononuclear cells from other components of whole blood, including red blood cells and polymorphonuclear cells (e.g., granulocytes), based on differential cell densities. The stabilized blood sample (or the cellular fraction thereof) is diluted with a dilution solution prior to performing the density gradient centrifugation step, preferably prior to contacting the stabilized blood sample (or the cellular fraction thereof) with the density gradient medium. Dilution may be at a ratio of at least 1:1. The diluted stabilized blood sample (or the diluted cellular fraction thereof) may be layered on top of the density gradient medium (preferred) or beneath it and is centrifuged to separate distinct cell populations from blood plasma, usually causing erythrocytes and granulocytes to pellet to the bottom of the tube and mononuclear cells (including rare cells such as CTCs), due to their lower density, to remain above the gradient-medium layer in an interphase layer where they are accessible for collection and analysis. However, as described herein and known in the art, the density of cell populations may be artificially altered to achieve that they settle in different cell-containing layers. E.g. use of the RosetteSep™ CTC Enrichment Cocktail (StemCell Technologies) in combination with Ficoll® separation allows for CTC enrichment by utilizing tetrameric antibody complexes which crosslink CD45-expressing leukocytes to red blood cells, thus artificially altering the density of labeled leukocytes and causing them to pellet to the bottom in order to enrich the interphase layer for CTCs.

As shown in the examples, the stabilization composition used according to the present disclosure in order to stabilize the blood sample may in embodiments wherein the stabilization agents (a) to (c) are used in combination result in that an altered layer pattern is provided after density gradient centrifugation. To avoid handling errors, it is advantageous to pre-treat the stabilized blood sample (or the cellular fraction thereof) to ensure that the stabilized blood sample (or the cellular fraction thereof) provides upon density gradient centrifugation a layer pattern that resembles the layer pattern of a common EDTA stabilized blood sample (or of a cellular fraction thereof). It was found that this can be achieved if the stabilized blood sample (or the cellular fraction thereof) is diluted with a dilution solution that is different from PBS which is commonly used. The dilution solution used may be a hypotonic solution or an isotonic solution as described herein. Dilution may be performed at a ratio of at least 1:1.

In one embodiment said dilution solution comprises a tonicity modifier. Tonicity modifiers are known in the art, and include compounds such as salts (e.g., sodium chloride, potassium chloride, calcium chloride, sodium phosphate, potassium phosphate, sodium bicarbonate, calcium carbonate, sodium lactate) and polyols, such as sugars (e.g., glucose, dextran, dextrose, lactose, trehalose) and sugar alcohols (e.g., glycerol, mannitol, sorbitol, xylitol). The dilution solution may comprise a polyol. The term “polyol” as used herein refers to a substance with multiple hydroxyl groups, and includes sugars (reducing and nonreducing sugars) and sugar alcohols. The polyol may comprise at least three, at least four or at least five hydroxyl groups. In certain embodiments, polyols have a molecular weight that is 600 Da (e.g., in the range from 120 to 400 Da). A “reducing sugar” is one that contains a free aldehyde or ketone group and can reduce metal ions or react covalently with lysine and other amino groups in proteins. A “nonreducing sugar” is one that lacks a free aldehyde or ketone group and is not oxidised by mild oxidising agents such as Fehling's or Benedict's solutions. Examples of reducing and nonreducing sugars are known to the skilled person. In embodiments, the comprised compound (tonicity modifier/polyol) is able to penetrate the cell membrane.

In embodiments, the comprised polyol that may act as tonicity modifier is a sugar or a sugar alcohol. Combinations of sugars and/or sugar alcohols may also be used. The sugar may be a reducing sugar or non-reducing sugar. In embodiments, the sugar is a reducing sugar. In embodiments, the dilution solution comprises glucose. In one embodiment, the dilution solution comprises a reducing sugar, optionally glucose, in a concentration that lies in a range of 2-10%, 3-7% or 4-6% (w/v). In further embodiments, the dilution solution comprises a sugar alcohol, optionally glycerol. In embodiments, the dilution solution comprises a salt. The salt may act as tonicity modifier. The salt may be an alkali metal salt, optionally a chloride salt such as sodium chloride. In embodiments the dilution solution comprises a sugar alcohol (such as glycerol) and a salt, optionally an alkali metal salt (such as sodium chloride). In one embodiment, the dilution solution comprises up to 0.5M glycerol and up to 2% sodium chloride, optionally wherein the dilution solution comprises 0.7-1.2% sodium chloride and 0.075-0.15M glycerol. In embodiments, the dilution solution is selected from (i) 5% (w/v) glucose, (ii) 0.9% NaCl+0.1 M glycerol, and (iii) a dilution solution comprising at least one tonicity modifier and having a osmolality that corresponds to the osmolality of the dilution solution defined in (i) or (ii), or wherein the osmolality is within a range of +/−20%, +/−15% or +/−10% of the osmolality of the solution as defined in (i) or (ii).

According to one embodiment, the dilution solution comprises DMSO. The dilution solution may comprise DMSO in a concentration of 1%-10% (v/v), e.g. 1%-5% (v/v).

In embodiments, the stabilized blood sample is incubated no longer than 10 min, no longer than 5 min or no longer than 3 min in the dilution solution before contacting the diluted stabilized blood sample (or cellular fraction thereof) with the density gradient medium. Preferably, the diluted stabilized blood sample (or cellular fraction thereof) is directly processed after dilution and contacted with the density gradient medium.

As is demonstrated in the examples, the use of such dilution solution advantageously restores the density of the stabilized blood cells and thereby ensures that after density gradient centrifugation, essentially the same layer types may be formed as are formed in EDTA-stabilized blood samples. After density gradient centrifugation, different layers are formed, wherein a distinct PBMC layer is formed. The formed layers may comprise (from top to bottom): a top layer (e.g. comprising plasma in case of a stabilized blood sample or comprising predominantly the dilution solution when processing the cellular fraction of a stabilized blood sample), a PBMC layer (also comprises CTCs, if present in the stabilized sample), a density gradient medium layer and furthermore the granulocytes and erythrocytes. A further layer may form below the granulocyte/erythrocyte layer. Important is the distinct formation of a PBMC layer, as this layer may be further processed as cell-subpopulation, e.g. for CTC analysis. In one embodiment, the method thus comprises collecting the formed PBMC layer thereby providing a PBMC fraction. The collected PBMC fraction may be washed. Washing may be performed using a buffer, optionally a PBS buffer or other suitable solution. The collected PBMC layer may be further processed and/or analysed. As disclosed in the background, the genomic and/or epigenomic profiling of peripheral mononuclear blood cells (PMBCs) represents a biomarker of interest for early diagnosis and monitoring of immunosurveillance in cancer patients. Furthermore, it may be used for enriching specific cell types therefrom, such as CTCs. The plasma fraction that may form on top of the PBMC layer in case a stabilized blood sample is subjected to density gradient centrifugation may also be further processed or may be discarded. Embodiments for processing plasma are described elsewhere herein.

In one embodiment, the method comprises using the collected PBMC fraction for enriching or detecting circulating tumor cells.

The so enriched biological targets may be further processed and analysed in step (D). E.g. genomic DNA may be purified from the collected PBMC fraction, from which circulating tumor cells were optionally depleted in advance. Furthermore, at least a fraction of the PBMC cells may be subjected to white blood cell counting or other analysis. Furthermore, specific cell types may be enriched from the collected PBMC fraction.

### Extracellular Nucleic Acids and Enrichment Extracellular Nucleic Acids

According to one embodiment, step (C) comprises obtaining a cell-depleted fraction from the stabilized cell-containing bodily fluid sample and enriching, in particular purifying, extracellular nucleic acids from the obtained cell-depleted fraction.

“Extracellular nucleic acids” or “extracellular nucleic acid” as used herein, in particular refers to nucleic acids that are not contained in cells but are comprised in the extracellular fraction of the cell-containing bodily fluid sample. Respective extracellular nucleic acids are also often referred to as cell-free nucleic acids. These terms are used as synonyms herein. Cell-free nucleic acids obtained from a circulating bodily fluid (such as blood) are also referred to as circulating cell-free nucleic acids, e.g. ccfDNA or ccfRNA. Extracellular nucleic acids may be enriched from the cell-depleted fraction that may be obtained from the cell-containing bodily fluid (e.g. blood plasma or serum, preferably plasma). The term “extracellular nucleic acids” refers e.g. to extracellular RNA as well as to extracellular DNA. Examples of typical extracellular nucleic acids that are found in the cell-free fraction of body fluids include but are not limited to mammalian extracellular nucleic acids such as e.g. extracellular tumor-associated or tumor-derived DNA and/or RNA, other extracellular disease-related DNA and/or RNA, epigenetically modified DNA, fetal DNA and/or RNA, small interfering RNA such as e.g. miRNA and siRNA, and non-mammalian extracellular nucleic acids such as e.g. viral nucleic acids, pathogen nucleic acids released into the extracellular nucleic acid population e.g. from prokaryotes (e.g. bacteria), viruses, eukaryotic parasites or fungi. The extracellular nucleic acid population usually comprises certain amounts of intracellular nucleic acids that were released from damaged or dying cells. E.g. the extracellular nucleic acid population present in blood usually comprises intracellular globin mRNA that was released from damaged or dying cells. This is a natural process that occurs in vivo. Such intracellular nucleic acid present in the extracellular nucleic acid population can even serve the purpose of a control in a subsequent nucleic acid detection method. The stabilization method described herein in particular reduces the risk that the amount of intracellular nucleic acids, such as genomic DNA, that is comprised in the extracellular nucleic acid population is significantly increased after the cell-containing bodily fluid was collected due to the ex vivo handling of the sample. Thus, alterations of the extracellular nucleic acid population because of the ex vivo handling are significantly reduced or even prevented with the stabilization technology according to the present disclosure.

The enriched, preferably purified, extracellular nucleic acids may preferably comprises or essentially consist of extracellular DNA. Extracellular DNA, such as ccfDNA (circulating cell-free DNA) obtained from a circulating bodily fluid, is a valuable tool for diagnostic applications and therefore widely used in the art for diagnostic and prognostic purposes.

In one embodiment, the isolated extracellular nucleic acids comprises or essentially consists of extracellular RNA. It is well-known and described in the art that the cell-depleted fraction obtained from a cell-containing bodily fluid sample (such as plasma in case of a stabilized blood sample) comprises extracellular RNA.

Suitable methods and kits for purifying extracellular nucleic acids are known in the art and also commercially available such as the QIAamp® Circulating Nucleic Acid Kit (QIAGEN), the QIAsymphony DSP Circulating DNA Kit, the Chemagic Circulating NA Kit (Chemagen), the NucleoSpin Plasma XS Kit (Macherey-Nagel), the Plasma/Serum Circulating DNA Purification Kit (Norgen Biotek), the Plasma/Serum Circulating RNA Purification Kit (Norgen Biotek), the High Pure Viral Nucleic Acid Large Volume Kit (Roche) and other commercially available kits suitable for extracting and purifying extracellular nucleic acids. It is furthermore referred to the methods disclosed in WO 2013/045432 and WO2016/198571. The described methods are particularly suitable for purifying extracellular nucleic acids, such as extracellular DNA, from plasma that was obtained from a blood sample that was stabilized using the stabilization method described herein.

In one embodiment, the extracellular nucleic acids are not isolated from pre-enriched extracellular vesicles, but from the cell-depleted fraction such as plasma or serum (preferably plasma) in case of blood.

In one embodiment, subsequent step (D) is performed and comprises detecting one or more target molecules within the extracellular nucleic acids that were purified in step (C).

### Extracellular Vesicles and Enrichment of Extracellular Vesicles

According to one embodiment, step (C) comprises enriching extracellular vesicles from a cell-depleted fraction obtained from the stabilized cell-containing bodily fluid sample.

The term extracellular vesicle (EV) as used herein in particular refers to any type of secreted vesicle of cellular origin. EVs may be broadly classified into exosomes, microvesicles (MVs) and apoptotic bodies. EVs such as exosomes and microvesicles are small vesicles secreted by cells. EVs have been found to circulate through many different body fluids including blood and urine which makes them easily accessible. Due to the resemblance of EVs composition with the parental cell, circulating EVs are a valuable source for biomarkers. Circulating EVs are likely composed of a mixture of exosomes and MVs. They contain nucleic acids (e.g. mRNA, miRNA, other small RNAs), DNA and protein, protected from degradation by a lipid bilayer. The contents are accordingly specifically packaged, and represent mechanisms of local and distant cellular communications. They can transport RNA between cells. EVs such as exosomes are an abundant and diverse source of circulating biomarkers. The cell of origin may be a healthy cell or a cancer cell. EVs such as exosomes are often actively secreted by cancer cells, especially dividing cancer cells. As part of the tumor microenvironment, EVs such as exosomes seem to play an important role in fibroblast growth, desmoplastic reactions but also initiation of epithelial-mesenchymal transition (EMT) and SC as well as therapy resistance building and initiation of metastases and therapy resistance. Exosomes are smaller than CTCs and comprise a lower number of copies per biomarker. Compared to CTCs, EVs are easier accessible because they are present in very large numbers in body fluids such as for example approx. 10⁹-10¹² vesicles per ml of blood plasma.

As discussed above, the present method comprises in one embodiment the enrichment of extracellular vesicles. Any method can be used in conjunction with the present method that is suitable to isolate and thus enrich extracellular vesicles from the stabilized cell-containing bodily fluid sample. As disclosed herein, the stabilized cell-containing bodily fluid sample may be first processed in order to provide a cell-depleted fraction, e.g. plasma in case of a stabilized blood sample. Different options for providing a cell-depleted fraction are disclosed herein. The extracellular vesicles may then be enriched from the cell-depleted fraction, such as the blood plasma. The term “enrichment” is again used in a broad sense and covers the enrichment or purification of extracellular vesicles. Extracellular vesicles can be enriched from virtually any biofluid after removing cellular components. Suitable methods for enriching extracellular vesicles such as exosomes are known in the art and therefore, need no detailed description herein. Exemplary suitable methods for enriching extracellular vesicles are briefly described herein.

Extracellular vesicles, including exosomes, can be enriched from the cell-depleted fraction of the stabilized bodily fluids, such as for example blood plasma or serum. E.g. extracellular vesicles may be enriched by ultracentrifugation, ultrafiltration, gradients and affinity capture or a combination of according methods. Numerous protocols and commercial products are available for extracellular vesicle/exosome isolation, and are known to the skilled person. Exemplary, non-limiting isolation methods are described in the following.

Extracellular vesicles and in particular exosomes can be enriched e.g. by methods involving ultracentrifugation. An exemplary ultracentrifugation isolation method is described by Thery et al. (Isolation and Characterization of Exosomes from Cell Culture Supernatants and Biological Fluids. Unit 3.22, Subcellular Fractionation and Isolation of Organelles, in Current Protocols in Cell Biology, John Wiley and Sons Inc., 2006). Hence according to one embodiment, extracellular vesicles are enriched by ultracentrifugation.

To increase the purity of the enriched extracellular vesicles, cells and cell fragments, and optionally apoptotic bodies if desired, can be removed prior to enriching the extracellular vesicles, e.g. by centrifugation or filtration. E.g. filtration methods can be used which exclude particles ≥0.8 μm, ≥0.7 μm or ≥0.6 μm.

According to one embodiment, extracellular vesicles are enriched by affinity capture to a solid phase. According to one embodiment, extracellular vesicles, such as exosomes, are enriched by immuno-magnetic capture using magnetic beads coated with antibodies directed against proteins exposed on extracellular vesicles, e.g. on exosomal membranes.

According to one embodiment, extracellular vesicles are captured by passing the cell-depleted sample through a vesicle capture material. Bound extracellular vesicles can be washed and subsequently eluted. Commercial systems that are based on affinity capture such as the exoEasy Kit (QIAGEN) are available for extracellular vesicle purification and can be used in conjunction with the present invention.

Methods based on the use of volume-excluding polymers, such as PEG, have also been described for the isolation of EVs. Therein, the polymers work by tying up water molecules and forcing less-soluble components such as extracellular vesicles out of solution, allowing them to be collected by a short, low-speed centrifugation. Commercial products that make use of this principle are ExoQuick (System Biosciences, Mountain View, USA) and Total Exosome Isolation Reagent (Life Technologies, Carlsbad, USA). Hence according to one embodiment, extracellular vesicles are enriched by precipitation with a volume-excluding polymer. Also, extracellular vesicles, such as exosomes, can be enriched based on their density, e.g. by layering the sample onto discontinuous sucrose or iodixanol gradients and subjecting to high speed centrifugation. Thus according to one embodiment, extracellular vesicles, such as exosomes, are enriched by density gradient centrifugation.

According to one embodiment, the extracellular vesicles comprise or predominantly consist of exosomes and/or microvesicles. According to one embodiment, the extracellular vesicles comprise or predominantly consist of exosomes. Thus, in embodiments, the enriched biological target essentially consists of exosomes.

As disclosed herein, the recovered extracellular vesicles may be further processed in step (D), e.g. in order to isolate nucleic acids, such as RNA, therefrom. RNA can thus be purified from the enriched extracellular vesicles, such as in particular enriched exosomes. Relevant molecular information may thus be obtained by analyzing RNA molecules present in extracellular vesicles such as exosomes. EVs have been shown to contain various small RNA species, including miRNA, piRNA, tRNA (and fragments thereof), vault RNA, Y RNA, fragments of rRNA, as well as long non-coding RNA, and also mRNA.

Exemplary and preferred methods for RNA isolation are described herein.

### Intracellular Nucleic Acids and Enrichment of Intracellular Nucleic Acids

According to one embodiment, step (C) comprises enriching, e.g. purifying, intracellular nucleic acids as biological target from the stabilized cell-containing bodily fluid sample. The intracellular nucleic acid may be purified from an aliquot of the stabilized cell-containing biological sample, or the stabilized cell-containing biological sample may be separated into a cell-containing and a cell-depleted fraction and intracellular nucleic acids may be purified from the cell-containing fraction, respectively an aliquot/portion thereof. Optionally, a target cell population, e.g. comprising or essentially consisting of rare cells may have been removed in advance, and intracellular nucleic acids may thus be enriched from the stabilized cell-containing bodily fluid and/or or a concentrated cell-containing fraction thereof, from which e.g. rare target cells have been depleted.

Furthermore, a subpopulation of cells may be first enriched from the stabilized cell-containing bodily fluid and intracellular nucleic acids are enriched from the sub-population. Suitable embodiments are described herein.

As disclosed herein, the cells may be enriched and thus concentrated in the cell-containing fraction. The intracellular nucleic acids may be selected from RNA and genomic DNA. According to one embodiment, genomic DNA is enriched as biological target. Thus, according to one embodiment, the method comprises obtaining a cellular fraction from the stabilized cell-containing bodily fluid sample and enriching genomic DNA from the cellular fraction, wherein the cellular fraction is stored, optionally frozen, prior to genomic DNA isolation.

Suitable method for purifying intracellular nucleic acids such as RNA and genomic DNA are well-known in the art and are also briefly described herein.

According to one embodiment, step (C) comprises enriching as biological targets at least circulating tumor cells, genomic DNA and circulating cell-free DNA.

### Step (D)

Step (D) comprises processing the enriched three or more biological targets for analysis. In particular, the analysis may comprise detection of one or more biomarker molecules.

According to one embodiment, step (C) comprises enriching a cell subpopulation, e.g. comprising or essentially consisting of rare cells (e.g. CTCs) and wherein subsequent step (D) comprises analysing the enriched cell subpopulation. Cell analysis may be important for fundamental cellular studies, drug discovery, diagnostics, and prognostics. The analysis may be conducted at the molecular level (DNA, RNA, protein, secreted molecules, etc.) or at the cellular level (cell metabolism, cell morphology, cell-cell interactions, etc.). Accordingly, subsequent step (D) may comprise analysing the enriched cell subpopulation (e.g. comprising or essentially consisting of rare cells such as CTCs) on a cellular level and/or enriching intracellular nucleic acids, e.g. RNA, from the enriched cell subpopulation. As disclosed herein, enriched rare cells preferably are circulating tumor cells.

Step (D) may accordingly comprise lysing the enriched cell subpopulation (e.g. comprising or essentially consisting of rare cells), in order to release intracellular nucleic acids for the subsequent purification. Suitable methods for purifying genomic DNA as well as RNA are known in the art and therefore, do not need to be described in detail.

According to one embodiment, step (D) comprises detecting one or more target molecules within the extracellular nucleic acids enriched in step (C).

According to one embodiment, step (C) comprises enriching extracellular vesicles from a cell-depleted fraction obtained from the stabilized cell-containing bodily fluid sample and wherein subsequent step (D) comprises enriching RNA from the enriched extracellular vesicles. As disclosed herein, the extracellular vesicles may comprise or essentially consist of exosomes.

According to one embodiment, step (D) comprises enriching RNA from cells, preferably from enriched rare cells, and/or from enriched extracellular vesicles. The enriched RNA may comprise or consist of mRNA and/or non-coding RNA. In embodiments, the purified RNA comprises miRNA or essentially consists of small RNA up to 350 nt in length, up to 300 nt in length or up to 250 nt length, which includes miRNA.

According to one embodiment, step (C) comprises, enriching as biological targets at least circulating tumor cells and circulating cell-free DNA and furthermore genomic DNA and/or extracellular vesicles and step (D) comprises
analysing the enriched circulating tumor cells, wherein analysing comprises enriching RNA from the enriched rare cells and detecting one or more target nucleic acid molecules within the enriched RNA (this e.g. allows to detect and/or characterize the enriched circulating tumor cells); and
detecting one or more target nucleic acid molecules within the circulating cell-free DNA.

Furthermore, in case genomic DNA was additionally enriched one or more target nucleic acid molecules may be detected within the genomic DNA. In case extracellular vesicles were additionally enriched, nucleic acids such as RNA may be enriched from the extracellular vesicles and one or more target nucleic acid molecules may be detected within the enriched nucleic acids.

In case platelets were enriched in step (C), nucleic acids such as RNA may be purified from the platelets and one or more target nucleic acid molecules may be detected within the purified nucleic acids in step (D).

Hence, according to a preferred embodiment, step (D) comprises detecting one or more target nucleic acid molecules within the isolated nucleic acids. Step (D) may comprise reverse transcribing isolated RNA to provide cDNA. Step (D) may furthermore comprise performing at least one amplification step (e.g. polymerase chain reaction, isothermal amplification, whole genome amplification etc.). According to one embodiment, step (D) comprises performing a qualitative or quantitative polymerase chain reaction. According to one embodiment, step (D) comprises performing a sequencing reaction. According to one embodiment step (D) comprises analyzing one or more intact cells, optionally wherein the cells are circulating tumor cells.

According to one embodiment, the at least one target nucleic acid molecule that is detected in step (D) has one or more of the following characteristics:
it is a cancer-associated tumor marker;
it is a diagnostic, prognostic and/or predictive biomarker;
it is a prognostic or predictive biomarker;
it is associated with a solid cancer, optionally a metastatic cancer;
it is associated with breast cancer or prostate cancer, in particular metastatic breast and metastatic prostate cancer;
it is a positive or negative response marker; and/or
it is a therapeutic marker.

According to one embodiment, the at least one target nucleic acid molecule forms part of a panel of target nucleic acid molecules. Therefore, step (D) may comprise detecting a panel of target nucleic acid molecules. A panel may comprise at least 5, at least 10, at least 15, at least 20, at least 25 or at least 50 target nucleic acid molecules. Detecting a panel of target nucleic acid molecules (e.g. using a corresponding panel of primers and optionally probes) is advantageous, e.g. in order to characterize enriched CTCs.

According to one embodiment, step (D) comprises isolating RNA from the circulating tumor cells and detecting biomarker RNA molecules in the isolated RNA.

In embodiments, step (D) comprises immunofluorescent staining of enriched cells. The enriched cells may be target cells, such as target rare cells. In embodiments, CTCs are analysed by immunofluorescent staining. Staining may be performed using e.g. mono- or polyclonal antibodies against markers specific for the target cells of interest to be stained. E.g. in case of CTCs, the cells may be stained for cytokeratins, Epcam, EGFR, E-cadherin, HER2, PSA, PSMA and/or other CTC markers. Furthermore, staining may involve staining of exclusion markers to exclude myeloid origin. Such markers may include CD45 and/or CD14.

### Enrichment of RNA

In embodiments, the present method comprises the enrichment, e.g. purification, of RNA from cells, such as rare cells (e.g. CTCs). The method may also comprise the isolation of RNA from extracellular vesicles. The term “enrichment” is again used in a broad sense and encompasses e.g. the isolation and purification of RNA. Suitable RNA isolation methods are known to the skilled person and therefore, do not need detailed description herein. Exemplary embodiments are briefly illustrated in the following.

Methods, e.g. based on the use of phenol and/or chaotropic salts, can be used for RNA isolation. Examples of suitable methods include, but are not limited to, extraction, solid-phase extraction, polysilicic acid-based purification, magnetic particle-based purification, phenol-chloroform extraction, anion-exchange chromatography (using anion-exchange surfaces), electrophoresis, precipitation and combinations thereof. According methods are well known in the art. In case DNA is enriched together with the RNA, DNA can be removed e.g. by DNase digestion. Methods are also known in the art that specifically isolate RNA, essentially free from DNA contaminations. As discussed, remaining DNA can moreover be removed by DNase digestion and/or intron spanning primers can be used in case expression of the biomarker RNA molecule is detected by amplification.

An example of a phenol/chloroform-based organic extraction method for the isolation of RNA is the Chomczynski method (Chomczynski and Sacchi, 1987: Single-step method of RNA isolation by acid guanidinium thiocyanate-phenol-chloroform extraction. Anal. Biochem. (162): 156-159) and variations thereof. An example of a phenol/chloroform based commercial product is the miRNeasy Mini kit (QIAGEN). It provides high quality and high yields of total RNA including small RNA from various different biological samples.

According to one embodiment, RNA isolation comprises binding RNA to a solid phase and eluting the RNA from the solid phase. The RNA may be washed prior to elution. Suitable solid phases and compatible chemistries to achieve RNA binding to the solid phase are known to the skilled person and include but are not limited to silica solid phases and solid phases with anion exchange moieties.

According to one embodiment, RNA isolation comprises binding RNA to a solid phase, such as in particular a silica solid phase, wherein at least one chaotropic agent (e.g. a guanidinium salt) and/or at least one alcohol (e.g. isopropanol or ethanol) are used for RNA binding. Suitable embodiments concentrations of chaotropic agents and alcohols are known to the skilled person. The bound RNA may optionally be washed and the RNA is eluted.

According to one embodiment, RNA isolation comprises binding RNA to a solid phase with anion exchange moieties and eluting the RNA from the solid phase. In particular, isolation methods that are based on the charge-switch principle may be used. Examples of suitable solid phases with anion exchange moieties comprise, but are not limited to, materials, such as particulate materials or columns, that are functionalized with anion exchange groups. Examples of anion exchange moieties are monoamines, diamines, polyamines, and nitrogen-containing aromatic or aliphatic heterocyclic groups. The RNA is bound to the solid phase at binding conditions that allow binding of the RNA to the anion exchange moieties. To that end, suitable pH and/or salt conditions can be used, as is known to the skilled person. The bound RNA can optionally be washed. Any suitable elution method can be used and suitable embodiments are known to the skilled person. Elution can e.g. involve changing the pH value. Thus, elution can e.g. occur at an elution pH which is higher than the binding pH. Likewise, ionic strength can be used to assist or effect the elution. Elution can also be assisted by heating and/or shaking.

The cells (e.g. the enriched CTCs) and/or the enriched extracellular vesicles can be lysed/digested to liberate the RNA from the cells or the extracellular vesicles for RNA isolation. Suitable lysis methods are well-known in the prior art. The cells and/or the extracellular vesicles can be contacted for disruption, respectively lysis, with one or more lysing agents. These can be contained in a disruption reagent such as a lysis buffer. RNA should be protected during lysis from degradation by nucleases. Generally, the lysis procedure may include but it is not limited to mechanical, chemical, physical and/or enzymatic actions on the sample. Furthermore, reducing agents such as beta-mercaptoethanol or DTT can be added for lysis to assist denaturation of e.g. nucleases. According to one embodiment, at least one chaotropic agent, such as preferably at least one chaotropic salt, is used for lysing and hence disruption. Suitable chaotropic agents and in particular suitable chaotropic salts are known to the skilled person.

According to one embodiment, an RNA fraction enriched in step (D) comprises or consists of mRNA. Step (D) encompasses the purification of RNA that comprises mRNA (among other RNA types) as well as the selective purification of mRNA. Essentially pure mRNA can be obtained e.g. by using RNA isolation methods which selectively isolate mRNA (but not other RNA types) from the digested sample. Purified mRNA can also be isolated sequentially, e.g. by first enriching total RNA, followed by selectively enriching mRNA from the isolated total RNA. Suitable methods for selective mRNA isolation are known to the skilled person and therefore, do not need detailed description. A well-established method is based on oligo(dT) capture to a solid phase (e.g. a column or magnetic beads), which allows to specifically isolates mRNA via its poly(A) tail. According to one embodiment, mRNA is isolated from the obtained cell lysate, e.g. from the rare cell lysate (such as a CTC lysate). According to one embodiment, mRNA is directly isolated from the obtained cell lysate, such as the CTC lysate as it is also shown in the examples. mRNA may be captured from the lysate using a solid phase (e.g. magnetic beads or a column) comprising oligo d(T) moieties (e.g. oligo d(T)₂₅ moieties). According to a further embodiment, total RNA is first isolated and mRNA is then isolated from the total RNA, e.g. by oligo d(T) capture or other suitable methods. According to one embodiment, total RNA is isolated from the obtained extracellular vesicle lysate/digest. According to one embodiment, mRNA is then isolated from the total vesicular RNA, e.g. by oligo d(T) capture or other suitable methods.

According to one embodiment, the RNA isolated in step (D) comprises miRNA or essentially consists of small RNA up to 350 nt in length, up to 250 nt length or up to 200 nt in length, which includes miRNA. Step (D) may thus encompass the purification of RNA that comprises miRNA (among other RNA types) as well as the specific purification of small RNA molecules that comprise miRNA but is depleted of large RNA molecules (e.g. having a length of 400 nt or larger). Suitable methods for enriching specifically small RNA molecules separately from large RNA molecules are well-known in the prior art and therefore, do not need to be described herein.

As disclosed herein, isolated RNA (such as mRNA) may be reverse transcribed into cDNA, followed by amplification. The amplification provides amplicons corresponding to the one or more target nucleic acid molecules tested for. Suitable primers for amplification can be determined by the skilled person. According to one embodiment, expression of two or more target nucleic acid molecules (e.g. biomarker RNAs) is determined in parallel by performing a multiplex-PCR using obtained cDNA as template. Suitable primers for amplification can be determined by the skilled person. Moreover, the reverse transcription step can be combined with an amplification step by performing e.g. a reverse transcription polymerase chain reaction. According to one embodiment, determining the expression of the at least one biomarker RNA molecule in the isolated RNA comprises performing a quantitative polymerase chain reaction. In one embodiment, a semi-quantitative PCR is performed. In another embodiment, the method is not semi-quantitative. Performing a quantitative PCR (qPCR) is advantageous because it allows to determine whether the biomarker RNA molecule is for example overexpressed in CTCs and/or EVs. Suitable methods for performing a quantitative PCR are well-known to the skilled person and therefore, need no detailed description herein. The Ct values obtained in the quantitative PCR for the individual one or more marker RNA molecules analysed can then be recorded and used for providing an expression profile. According to one embodiment, a pre-amplification step is performed after the reverse transcription step and prior to performing a quantitative PCR reaction. Such pre-amplification step can improve the sensitivity. This can be advantageous considering that CTCs are often rare. By pre-amplifying the cDNA molecules that correspond to the analyzed target nucleic acid molecule(s) (e.g. one or more biomarker RNA molecules) more DNA material is provided for the subsequent amplification step, which preferably is a qPCR. This can improve the results.

### Cell-Containing Bodily Fluid Samples

Advantageously, the cell-containing bodily fluid sample may be a liquid biopsy sample. The cell-containing bodily fluid is in one embodiment a circulating bodily fluid. The cell-containing bodily fluid may be selected from blood, urine, saliva, synovial fluids, amniotic fluid, lachrymal fluid, lymphatic fluid, liquor (cerebrospinal fluid), sweat, ascites, milk, bronchial lavage, peritoneal effusions and pleural effusions, bone marrow aspirates and nipple aspirates, semen/seminal fluid, body secretions or body excretions. The cell-containing bodily fluid may also be a product of diagnostic leukapheresis. In one embodiment, the cell-containing bodily fluid is selected from blood and urine. In one embodiment, it is blood. In one embodiment, the blood is peripheral blood.

The present method can be performed as in vitro method using a biological sample that has been obtained from a subject, e.g. a human subject such as a cancer patient. In one embodiment where at least one biological target is rare cells (e.g. tumor cells, such as CTCs), the cell-containing bodily fluid comprises or is suspected of comprising such rare cells.

As is demonstrated by the examples, rare cells, such as circulating tumor cells, extracellular nucleic acids (such as ccfDNA), extracellular vesicles such as exosomes and intracellular nucleic acids of the cellular fraction or a specific subpopulation thereof can be enriched from the same stabilized sample (e.g. blood sample) and analyzed with the present method. The described workflows enable the parallel analysis of multiple different biological targets that may be enriched from the same stabilized cell-containing bodily fluid.

### The Stabilization Technology Used According to the Present Disclosure

As disclosed above, step (A) comprises contacting a cell-containing bodily fluid with a stabilizing composition which comprises one or more, two or more, or preferably all three of the following stabilizing agents:

(a) at least one primary, secondary or tertiary amide,
(b) at least one poly(oxyethylene) polymer, and/or
(c) at least one apoptosis inhibitor.

Thereby, a stabilized cell-containing bodily fluid sample is provided.

Suitable embodiments and concentrations for the stabilizing agents (a) to (c) as well as advantageous embodiments of the stabilizing composition are disclosed e.g. in WO2015/140218, herein incorporated by reference. Suitable embodiments are also briefly described below.

### The at Least One Primary, Secondary or Tertiary Amide

According to one embodiment, the stabilization composition comprises at least one primary, secondary or tertiary amide. As disclosed herein, the amide may be a carboxylic acid amide, a thioamide or a selenoamide. Preferably, it is a carboxylic acid amide.

According to one embodiment, the composition accordingly comprises one or more compounds according to formula 1

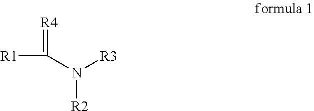

wherein R1 is a hydrogen residue or an alkyl residue, preferably a C1-C5 alkyl residue, a C1-C4 alkyl residue or a C1-C3 alkyl residue, more preferred a C1-C2 alkyl residue, R2 and R3 are identical or different and are selected from a hydrogen residue and a hydrocarbon residue, preferably an alkyl residue, with a length of the carbon chain of 1-20 atoms arranged in a linear or branched manner, and R4 is an oxygen, sulphur or selenium residue, preferably R4 is oxygen.

Also a combination of one or more compounds according to formula 1 can be used. In embodiments, wherein R1 is an alkyl residue, a chain length of 1 or 2 is preferred for R1. R2 and/or R3 of the compound according to formula 1 are identical or different and are selected from a hydrogen residue and a hydrocarbon residue, which preferably is an alkyl residue. According to one embodiment, R2 and R3 are both hydrogen. According to one embodiment, one of R2 and R3 is a hydrogen and the other is a hydrocarbon residue. According to one embodiment, R2 and R3 are identical or different hydrocarbon residues. The hydrocarbon residues R2 and/or R3 can be selected independently of one another from the group comprising alkyl, including short chain alkyl and long-chain alkyl, alkenyl, alkoxy, long-chain alkoxy, cycloalkyl, aryl, haloalkyl, alkylsilyl, alkylsilyloxy, alkylene, alkenediyl, arylene, carboxylates and carbonyl (regarding these residues see e.g. WO 2013/045457, p. 20 to 21, herein incorporated by reference). The chain length n of R2 and/or R3 can in particular have the values 1, 2, 3, 4, 5, 6, 7, 8, 9, 10, 11, 12, 13, 14, 15, 16, 17, 18, 19 and 20. According to one embodiment, R2 and R3 have a length of the carbon chain of 1-10, preferably 1 to 5, more preferred 1 to 2. According to one embodiment, R2 and/or R3 are alkyl residues, preferably C1-C5 alkyl residues. Preferably, the compound according to formula 1 is a carboxylic acid amide so that R4 is oxygen. It can be a primary, secondary or tertiary carboxylic acid amide.

According to one embodiment, the compound according to formula 1 is a N,N-dialkyl-carboxylic acid amide. Preferred R1, R2, R3 and R4 groups are described above. According to one embodiment, the compound according to formula 1 is selected from the group consisting of N,N-dimethylacetamide, N,N-diethylacetamide, N,N-dimethylformamide and N,N-diethylformamide. Also suitable are the respective thio analogues, which comprise sulphur instead of oxygen as R4. Preferably, at least one compound according to formula 1 is used which is not a toxic agent according to the GHS classification. According to one embodiment, the compound according to formula 1 is a N,N-dialkylpropanamide, such as N, N-dimethylpropanamide.

The stabilizing composition may comprise one or more compounds according to formula 1′

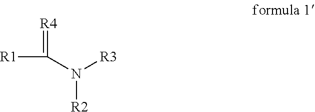

wherein R1 is a hydrogen residue or an alkyl residue, preferably a C1-C5 alkyl residue, more preferred a methyl residue, R2 and R3 are identical or different hydrocarbon residues with a length of the carbon chain of 1-20 atoms arranged in a linear or branched manner, and R4 is an oxygen, sulphur or selenium residue. Formula 1′ is encompassed by Formula 1 discussed above and is compared thereto limited in that R2 and R3 are identical or different hydrocarbon residues (not hydrogen). Otherwise, the residues R1 to R4 correspond to the ones discussed above for Formula 1 and it is referred to the above disclosure which also applies here.

Preferably, the composition comprises butanamide and/or a N,N-dialkylpropanamide, more preferably N,N-dimethlypropanamide.

According to one embodiment, the stabilization composition comprises one or more primary, secondary or tertiary amides in a concentration selected from 0.4% to 38.3%, 0.8% to 23.0%, 2.3% to 11.5%, 3.8% to 9.2%, 5% to 15% and 7.5% to 12.5%. The aforementioned concentrations refer to (w/v) or (v/v) depending on whether the primary, secondary or tertiary amide is a liquid or not. The use of at least one primary, secondary or tertiary carboxylic acid amide is preferred. According to one embodiment, the cell-containing bodily fluid sample is contacted with the stabilizing composition which comprises the one or more primary, secondary or tertiary amide (and optionally further additives used for stabilization) and the resulting mixture/stabilized cell-containing bodily fluid sample comprises said amide (or combination of amides) in a concentration range that lies in a range of 0.25% to 5%, such as 0.3% to 4%, 0.4% to 3%, 0.5% to 2% or 0.75% to 1.5%.

### The at Least One Poly(Oxyethylene) Polymer

According to one embodiment, the stabilization composition comprises at least one poly(oxyethylene) polymer. As it is described in detail in WO2015/140218 to which it is referred, poly(oxyethylene) polymers exhibit advantageous stabilization properties. Therefore, it is advantageous that the stabilization composition includes a poly(oxyethylene) polymer.

The poly(oxyethylene) polymer is preferably a polyethylene glycol. Unsubstituted polyethylene glycol may be used. All disclosures described in this application for the poly(oxyethylene) polymer in general, specifically apply and particularly refer to the preferred embodiment polyethylene glycol even if not explicitly stated. The poly(oxyethylene) polymer can be used in various molecular weights. The polyethylene glycol may be of the formula HO—(CH₂CH₂O)ₙ—H, wherein n is a whole integer and depends on the molecular weight.

A correlation was found between the stabilization effect of the poly(oxyethylene) polymer and its molecular weight. Higher molecular weight poly(oxyethylene) polymers were found to be more effective stabilizing agents than lower molecular weight poly(oxyethylene) polymers. To achieve an efficient stabilization with a lower molecular weight poly(oxyethylene) polymer, generally higher concentrations are recommendable compared to a higher molecular weight poly(oxyethylene) polymer. However, for several applications it is preferred though to keep the amount of additives used for stabilization low. Therefore, in embodiments, a higher molecular weight poly(oxyethylene) polymer is used as stabilizing agent, as it allows to use lower concentrations of the poly(oxyethylene) polymer while achieving a strong stabilization effect on the cell-containing bodily fluid sample and the biological targets of interest comprised therein.

According to one embodiment, the stabilizing composition comprises a poly(oxyethylene) polymer which is a high molecular weight poly(oxyethylene) polymer having a molecular weight of at least 1500. The comprised high molecular weight poly(oxyethylene) polymer may have a molecular weight that lies in a range selected from 1500 to 50000, 1500 to 40000, 2000 to 30000, 2500 to 25000, 3000 to 20000, 3500 to 15000 and 4000 to 12500. Alternatively or additionally, the stabilizing composition comprises at least one poly(oxyethylene) polymer having a molecular weight below 1500, preferably a low molecular weight poly(oxyethylene) polymer having a molecular weight of 1000 or less. In one embodiment, the molecular weight of the low molecular weight poly(oxyethylene) polymer lies in a range selected from 100 to 1000, 200 to 800, 200 to 600 and 200 to 500.

According to one embodiment, the stabilization composition that is contacted with the cell-containing bodily fluid in step (A) comprises a high molecular weight poly(oxyethylene) polymer which preferably is a polyethylene glycol in a concentration selected from 0.4% to 35% (w/v), such as 0.8% to 25% (w/v), 1.5% to 20% (w/v), 2.5% to 17.5% (w/v), 3% to 15% (w/v), 4% to 10% (w/v) or 3% to 5% (w/v). Suitable concentrations can be determined by the skilled person and may inter alia depend on whether the high molecular weight poly(oxyethylene) glycol is used as alone or in combination with a further poly(oxyethylene) polymer such as a low poly(oxyethylene) polymer and the amount, e.g. the volume, of the stabilization composition used to stabilize a certain amount of cell-containing bodily fluid sample. The high molecular weight poly(oxyethylene) polymer alone may be used in a concentration within a range of 2.2% to 33.0% (w/v). Suitable concentration ranges may be selected from 4.4% to 22.0 (w/v) %, 6.6% to 16.5% (w/v) and 8.8% to 13.2% (w/v). When using a high molecular weight poly(oxyethylene) polymer in combination with a low molecular weight poly(oxyethylene) polymer the concentration may be within a range of 0.4% to 30.7% (w/v). Suitable concentration ranges may be selected from 0.8% to 15.3% (w/v), 1% to 10% (w/v), 1.5% to 7.7% (w/v), 2.5% to 6% (w/v), 3.1% to 5.4% (w/v) and 3% to 4% (w/v).

According to one embodiment, the cell-containing bodily fluid sample is contacted with the stabilizing composition which comprises a high molecular weight poly(oxyethylene) polymer (and optionally further additives used for stabilization) and the resulting mixture/stabilized cell-containing bodily fluid sample comprises the high molecular weight poly(oxyethylene) polymer in a concentration range that lies in a range of 0.05% to 4% (w/v), such as 0.1% to 3% (w/v), 0.2% to 2.5% (w/v), 0.25% to 2% (w/v), 0.3% to 1.75% (w/v) and 0.35% to 1.5% (w/v). The concentration of the high molecular weight poly(oxyethylene) polymer in the stabilized cell-containing bodily fluid sample may be in a range of 0.25% to 1.5% (w/v), such as in the range of 0.3% to 1.25% (w/v), 0.35% to 1% (w/v) or 0.4% to 0.75% (w/v).

According to one embodiment, the stabilization composition comprises a low molecular weight poly(oxyethylene) polymer, which preferably is a polyethylene glycol, in a concentration within a range of 0.8% to 92.0%, such as 3.8% to 76.7%, 11.5% to 53.7%, 19.2% to 38.3%, 20% to 30% or 20% to 27.5%. According to one embodiment, the concentration is from 11.5% to 30%. The aforementioned concentrations refer to (w/v) or (v/v) depending on whether the low molecular weight poly(oxyethylene) polymer is a liquid or not.

According to one embodiment, the cell-containing bodily fluid sample is contacted with the stabilizing composition which comprises a low molecular weight poly(oxyethylene) polymer (and optionally further additives used for stabilization) and the resulting mixture/stabilized cell-containing bodily fluid sample comprises the low molecular weight poly(oxyethylene) polymer in a concentration range that lies in a range of 0.5% to 10%. The concentration of the low molecular weight poly(oxyethylene) polymer in the stabilized cell-containing bodily fluid sample may be in a range of 1.5% to 9%, such as in the range of 2% to 8%, 2 to 7%, 2.5% to 7% and 3% to 6%.

In one embodiment, the stabilizing composition comprises a poly(oxyethylene) polymer which is a high molecular weight poly(oxyethylene) polymer having a molecular weight of at least 1500 and comprises a low molecular weight poly(oxyethylene) polymer having a molecular weight of 1000 or less. In one embodiment, the stabilizing composition comprises a poly(oxyethylene) polymer which is a high molecular weight poly(oxyethylene) polymer and a poly(oxyethylene) polymer which is a low molecular weight poly(oxyethylene) polymer, wherein said high molecular weight poly(oxyethylene) polymer has a molecular weight that lies in a range selected from 1500 to 50000, 2000 to 40000, 3000 to 30000, 3000 to 25000, 3000 to 20000 and 4000 to 15000 and wherein said low molecular weight poly(oxyethylene) polymer has a molecular weight that lies in a range selected from 100 to 1000, 200 to 800, 200 to 600 and 200 to 500. Suitable concentrations were described above.

### The at Least One Apoptosis Inhibitor

According to one embodiment, the stabilization composition comprises at least one apoptosis inhibitor. Preferably, the apoptosis inhibitor is a caspase inhibitor. Suitable apoptosis inhibitors and caspase inhibitors are described in WO 2013/045457 A1 and WO 2013/045458 A1. The caspase inhibitors disclosed therein are incorporated herein by reference. Advantageous stabilizing compositions comprising one or more caspase inhibitors that can be used in the method according to the present disclosure are also disclosed in WO 2014/146780 A1, WO 2014/146782 A1, WO 2014/049022 A1, WO 2014/146781 A1, WO2015/140218 and WO 2017/085321.

Preferably, the caspase inhibitor is cell-permeable. Members of the caspase gene family play a significant role in apoptosis. The substrate preferences or specificities of individual caspases have been exploited for the development of peptides that successfully compete caspase binding. It is possible to generate reversible or irreversible inhibitors of caspase activation by coupling caspase-specific peptides to e.g. aldehyde, nitrile or ketone compounds. E.g. fluoromethyl ketone (FMK) derivatized peptides such as Z-VAD-FMK act as effective irreversible inhibitors with no added cytotoxic effects. Inhibitors synthesized with a benzyloxycarbonyl group (BOC) at the N-terminus and O-methyl side chains exhibit enhanced cellular permeability. Further suitable caspase inhibitors are synthesized with a phenoxy group at the C-terminus. An example is Q-VD-OPh which is a cell permeable, irreversible broad-spectrum caspase inhibitor that is even more effective in preventing apoptosis and thus supporting the stabilization than the caspase inhibitor Z-VAD-FMK.

According to one embodiment, the caspase inhibitor is a pancaspase inhibitor and thus is a broad spectrum caspase inhibitor. According to one embodiment, the caspase inhibitor comprises or consists of peptides or proteins. According to one embodiment, the caspase inhibitor comprises a modified caspase-specific peptide. Preferably, said caspase-specific peptide is modified by an aldehyde, nitrile or ketone compound. According to one embodiment, the caspase specific peptide is modified, preferably at the carboxyl terminus, with an O-Phenoxy (OPh) or a fluoromethyl ketone (FMK) group. Suitable caspase inhibitors comprising or consisting of proteins or peptides, and caspase inhibitors comprising modified caspase-specific peptides are disclosed in Table 1 of WO 2013/045457, and are incorporated herein by reference. The table provides examples of caspase inhibitors. In one embodiment, the caspase inhibitor is a peptidic caspase inhibitor that is modified, preferably at the carboxyl terminus, with an O-Phenoxy (OPh) group and/or is modified, preferably at the N-terminus, with a glutamine (Q) group. In one embodiment, the comprised caspase inhibitor is Q-VD-OPh.

According to one embodiment, the caspase inhibitor is selected from the group consisting of Q-VD-OPh, Z-VAD(OMe)-FMK and Boc-D-(OMe)-FMK. According to one embodiment, the caspase inhibitor is selected from the group consisting of Q-VD-OPh and Z-VAD(OMe)-FMK. In a preferred embodiment, Q-VD-OPh, which is a broad spectrum inhibitor for caspases, is used for stabilization. Q-VD-OPh is cell permeable and inhibits cell death by apoptosis. Q-VD-OPh is not toxic to cells even at extremely high concentrations and comprises a carboxy terminal phenoxy group conjugated to the amino acids valine and aspartate. It is equally effective in preventing apoptosis mediated by the three major apoptotic pathways, caspase-9 and caspase-3, caspase-8 and caspase-10, and caspase-12 (Caserta et al., 2003).

The stabilization composition that is used in step (A) may comprises one or more caspase inhibitors, in particular a caspase inhibitor comprising a modified caspase-specific peptide such as Q-VD-OPh, in an amount sufficient to yield a stabilization effect on the extracellular nucleic acid population that is contained in the biological sample. According to one embodiment, the stabilization composition comprises the caspase inhibitor in a concentration to yield a final concentration of 0.1 μM to 25 μM, 0.5 μM to 20 μM, 1 μM to 17 μM, 2 μM to 16 μM, more preferred 3 μM to 15 μM of caspase inhibitor after the stabilization composition has been contacted with the intended volume of the cell-containing biological bodily fluid to be stabilized. Final concentrations of in the range of 5 μM to 15 μM are well suitable e.g. for the stabilization of blood samples.

According to one embodiment, the stabilization composition and hence the stabilization reagent comprises the caspase inhibitor in a concentration selected from 0.35 μg/ml to 70 μg/ml, 0.7 μg/ml to 63 μg/ml, 1.74 μg/ml to 59 μg/ml, 10.5 μg/ml to 56 μg/ml, or 15 μg/ml to 50 μg/ml, 20 μg/ml to 45 μg/ml, 25 μg/ml to 40 μg/ml and 30 μg/ml to 38 μg/ml. The concentration can be selected from 0.7 μg/ml to 45 μg/ml and 1.74 μg/ml to 40 μg/ml. According to one embodiment, the stabilization composition and hence the stabilization reagent comprises the caspase inhibitor in a concentration selected from 0.68 μM to 136 μM, 1.36 μM to 122.5 μM, 3.38 μM to 114.72 μM, 20.4 μM to 109 μM, or 29.2 μM to 97.2 μM, 38.9 μM to 87.5 μM, 48.6 μM to 77.8 μM and 58.3 μM to 74 μM. The concentration can be selected from 20.4 μM to 97.2 μM and 29.2 μM to 87.5 μM.

The above mentioned concentrations of the caspase inhibitor in the mixture comprising the stabilization composition (reagent) and the cell-containing bodily fluid to be stabilized and the stabilization composition (reagent) as such apply to the use of a single caspase inhibitor as well as to the use of a combination of caspase inhibitors. The aforementioned concentrations are in particular suitable when using a pancaspase inhibitor, in particular a modified caspase specific peptide such as Q-VD-OPh and/or Z-VAD(OMe)-FMK. A further example of a modified caspase specific peptide is Boc-D-(OMe)-FMK. The above mentioned concentrations are e.g. suitable for stabilizing blood samples. Suitable concentration ranges for individual caspase inhibitors and/or for other cell-containing biological samples can be determined by the skilled person, e.g. by testing different concentrations of the respective caspase inhibitor in the test assays described in the examples.

### Further Components of the Stabilizing Composition

The cell-containing bodily fluid may also be contacted with further additives, which are preferably comprised in the stabilizing composition.

According to one embodiment, a further additive is a chelating agent. A chelating agent is an organic compound that is capable of forming coordinate bonds with metals through two or more atoms of the organic compound. Chelating agents include, but are not limited to ethylenedinitrilotetraacetic acid (EDTA), diethylenetriaminepentaacetic acid (DTPA), ethylene glycol tetraacetic acid (EGTA) and N,N-bis(carboxymethyl)glycine (NTA) and furthermore, salts of carboxylic acids such as citrate or oxalate. According to a preferred embodiment, EDTA is used as chelating agent. As used herein, the term “EDTA” indicates inter alia the EDTA portion of an EDTA compound such as, for example, K₂EDTA, K₃EDTA or Na₂EDTA. Using a chelating agent such as EDTA also has the advantageous effect that nucleases such as DNases and RNases are inhibited, thereby e.g. preventing a degradation of extracellular nucleic acids by nucleases. EDTA used/added in higher concentrations supports the stabilizing effect.

In case the cell-containing bodily fluid sample is blood, an anticoagulant is used as further additive. Anticoagulants include but are not limited to heparin, chelating and salts of carboxylic acids such as citrate or oxalate. In an advantageous embodiment, the anticoagulant is a chelating agent such as EDTA. E.g. K₂EDTA may be used. This embodiment is particularly useful in case the bodily fluid to be stabilized is blood.

According to one embodiment, a further additive is at least one compound selected from a thioalcohol that is N-acetyl-cysteine or glutathione, a water-soluble vitamin, and a water-soluble vitamin E derivate. As disclosed in WO 2017/085321 this can be advantageous in case the stabilizing composition additionally comprises a caspase inhibitor and is provided in sterilized form.

According to one embodiment, the used stabilization technology has one or more of the following characteristics:
(i) the stabilization of the cell-containing body fluid sample does not involve the use of additives in a concentration wherein said additives would induce or promote lysis of nucleated cells;
(ii) the stabilization does not induce protein-nucleic acids or protein-protein cross-links;
(iii) the stabilization does not involve the use of a cross-linking agent that induces protein-nucleic acid and/or protein-protein crosslinks, such as formaldehyde, formaline, paraformaldehyde or a formaldehyde releaser;
(iv) the stabilization does not involve the use of toxic agents; and/or
(v) the stabilizing agents are contained in an stabilization composition comprising water.

In particular, it is preferred that the stabilizing composition used for providing the stabilized cell-containing bodily fluid sample does not comprise a cross-linking agent that induces protein-DNA and/or protein-protein crosslinks. A cross-linking agent that induces protein-DNA and/or protein-protein crosslinks is e.g. formaldehyde, formalin, paraformaldehyde or a formaldehyde releaser. Crosslinking reagents cause inter- or intra-molecular covalent bonds between nucleic acid molecules or between nucleic acids and proteins. This effect can lead to a reduced recovery of such stabilized and partially crosslinked nucleic acids after a purification or extraction from a complex biological sample. As, for example, the concentration of circulating nucleic acids in a whole blood samples is already relatively low, any measure which further reduces the yield of such nucleic acids should be avoided. This may be of particular importance when detecting and analyzing very rare nucleic acid molecules derived from malignant tumors or from a developing fetus in the first trimester of pregnancy. Therefore, it is preferred that no formaldehyde releaser is comprised in the sterilized stabilizing composition, respectively is not additionally used for stabilization. Thus, according to one embodiment, no cross-linking agents such as formaldehyde or formaldehyde releasers are comprised in the stabilizing composition, respectively are not additionally used for stabilization. Furthermore, as described, the stabilizing composition does preferably not comprise any additives that would induce the lysis of nucleated cells or cells in general, such as e.g. chaotropic salts. As is demonstrated in the examples, this is an important advantage over known state-of-the-art stabilization reagents and methods which involve the use of cross-linking reagents, such as formaldehyde, formaldehyde releasers and the like, as it allows the efficient recovery of biological targets of interest (such as CTCs, extracellular nucleic acids, cell subpopulations and intracellular nucleic acids) from the stabilized cell-containing bodily fluid sample.

To use a stabilization composition that does not contain a component that is capable of releasing an aldehyde is advantageous. This can avoid impairment of the subsequent nucleic acid isolation from the stabilized sample.

### Advantageous Combinations of Stabilizing Agents in the Stabilizing Composition

According to one embodiment, the used stabilizing composition comprises:
(a) at least one primary, secondary or tertiary amide, and
(b) at least one poly(oxyethylene) polymer, preferably a high molecular weight polyethylene glycol and a low molecular weight polyethylene glycol; and
(c) optionally at least one apoptosis inhibitor, preferably a caspase inhibitor.

According to one embodiment, the used stabilizing composition comprises:
(a) at least one primary, secondary or tertiary amide;
(b) optionally at least one poly(oxyethylene) polymer;
(c) at least one apoptosis inhibitor, preferably a caspase inhibitor.

According to one embodiment, the used stabilizing composition comprises:
(a) optionally at least one primary, secondary or tertiary amide;
(b) at least one poly(oxyethylene) polymer;
(c) at least one apoptosis inhibitor, preferably a caspase inhibitor.

According to one embodiment, the used stabilizing composition comprises:
(a) at least one primary, secondary or tertiary amide;
(b) at least one poly(oxyethylene) polymer;
(c) at least one caspase inhibitor.

Suitable and preferred embodiments for the individual stabilizing agents (a) to (c) as well as suitable and preferred concentrations are described above.

According to one embodiment, the cell-containing bodily fluid, which preferably is blood, is contacted with:
a) one or more compounds according to formula 1 above;
b) at least one high molecular weight poly(oxyethylene) polymer having a molecular weight of at least 3000 and optionally at least one low molecular weight poly(oxyethylene) polymer having a molecular weight of 1000 or less;
c) at least one caspase inhibitor; and
d) optionally a chelating agent, preferably EDTA.

According to one embodiment, blood is contacted with:
a) one or more compounds according to formula 1 above;
b) at least one high molecular weight poly(oxyethylene) polymer having a molecular weight that lies in a range of 3000 to 40000, such as in a range of 3000 to 30000 or 3500 to 25000 and at least one low molecular weight poly(oxyethylene) polymer having a molecular weight of 1000 or less, such as in a range of 100 to 800, 200 to 800 or 200 to 500;
c) at least one caspase inhibitor, preferably a pancaspase inhibitor, optionally Q-VD-OPh; and
d) an anticoagulant which preferably is a chelating agent, preferably EDTA,
   wherein after the blood sample has been contacted with said additives and optionally further additives used for stabilization the resulting mixture/stabilized blood sample comprises
the one or more compounds according to formula 1 in a concentration that lies in a range of 0.3% to 4%, such as 0.5 to 3%, 0.5 to 2% or 0.75 to 1.5%,
the high molecular weight poly(oxyethylene) polymer in a concentration that lies in a range of 0.2% to 1.5% (w/v), such as 0.25% to 1.25% (w/v), 0.3% to 1% (w/v) or 0.4% to 0.75% (w/v),
the low molecular weight poly(oxyethylene) polymer in a concentration that lies in the range of 1.5% to 10%, such as 2% to 6%, and
the caspase inhibitor in a concentration that lies in a range of 1 μM to 10 μM, such as 3 μM to 7.5 μM.

The stabilization composition can be a liquid. The indicated concentrations are particularly preferred for the stabilisation of blood samples. E.g. a liquid stabilisation composition of 0.5 ml to 2.5 ml, 0.5 ml to 2 ml, preferably 1 ml to 2 ml or 1 ml to 1.5 ml can be used. Such stabilization composition comprising the stabilizing agents in the concentrations indicated below, can be used for stabilizing e.g. 10 ml blood.

### SPECIFIC EMBODIMENTS

Further embodiments of the present invention are described again in the following. The present invention in particular also provides for the following items:

1. A method for stabilizing and enriching multiple biological targets comprised in a cell-containing bodily fluid, said method comprising
(A) contacting a cell-containing bodily fluid with a stabilizing composition comprising one or more of the following stabilizing agents:
   (a) at least one primary, secondary or tertiary amide,
   (b) at least one poly(oxyethylene) polymer, and/or
   (c) at least one apoptosis inhibitor, thereby providing a stabilized cell-containing bodily fluid sample;
(B) keeping the stabilized cell-containing bodily fluid sample for a stabilization period; and
(C) processing the stabilized cell-containing bodily fluid sample in order to enrich three or more biological targets selected from the group consisting of
   at least one cell subpopulation,
   extracellular nucleic acids,
   extracellular vesicles, and
   intracellular nucleic acids
   from the stabilized cell-containing bodily fluid.

2. The method according to embodiment 1, wherein the enriched cell subpopulation comprises target rare cells.

3. The method according to embodiment 1 or 2, wherein the cell subpopulation essentially consists of the target rare cells.

4. The method according to any one of embodiments 1 to 3, wherein the target rare cells are selected from the group consisting of tumor cells, in particular circulating tumor cells (CTCs), fetal cells, stem cells, cells infected by a virus or parasite, circulating endothelial cells (CECs) and circulating endothelial progenitor cells (EPCs).

5. The method according to any one of embodiments 1 to 4, wherein the target rare cells are circulating tumor cells.

6. The method according to one or more of embodiments 1 to 5, wherein intracellular nucleic acids are isolated from the stabilized bodily fluid sample or a cell-containing fraction thereof, optionally wherein the intracellular nucleic acids is genomic DNA.

7. The method according to one or more of embodiments 1 to 6, wherein step (C) comprises processing the stabilized cell-containing bodily fluid sample in order to enrich three or more biological targets selected from the group consisting of
rare cells, preferably circulating tumor cells,
extracellular nucleic acids,
extracellular vesicles and
intracellular nucleic acids
   from the stabilized cell-containing bodily fluid.

8. The method according to one or more of embodiments 1 to 7, wherein step (C) comprises obtaining at least one cell-containing fraction and at least one cell-depleted fraction from the stabilized bodily fluid sample, optionally wherein a cell-depleted fraction is separated from at least one cellular fraction by a separation method involving centrifugation and/or filtration.

9. The method according to any one of embodiments 1 to 8, wherein processing in (C) comprises
(aa) separating the stabilized cell-containing bodily fluid sample into at least one cell-containing fraction and at least one cell-depleted fraction;
(bb) further processing the cell-containing fraction, wherein further processing the cell-containing fraction comprises
   (i) enriching a cell subpopulation, preferably comprising target rare cells, from the cell-containing fraction; and/or
   (ii) enriching intracellular nucleic acids (e.g. genomic DNA) from the cell-containing fraction;
(cc) further processing the cell-depleted fraction, wherein further processing the cell-depleted fraction comprises
   (i) enriching extracellular nucleic acids, optionally extracellular DNA, from the cell-depleted fraction; and/or
   (ii) enriching extracellular vesicles from the cell-depleted fraction.

10. The method according to any one of embodiments 1 to 8, wherein processing in (C) comprises
(aa) enriching a cell subpopulation, preferably comprising target rare cells, from the stabilized cell-containing bodily fluid sample;
(bb) separating the stabilized cell-containing bodily fluid sample from which the target cell subpopulation was removed into a cell-containing fraction and a cell-depleted fraction;
(cc) further processing the cell-depleted fraction, wherein further processing the cell-depleted fraction comprises
   (i) enriching extracellular nucleic acids, optionally extracellular DNA, from the cell-depleted fraction; and/or
   (ii) enriching extracellular vesicles from the cell-depleted fraction; and
(dd) optionally enriching intracellular nucleic acids, preferably genomic DNA, from the cell-containing fraction.

11. The method according any one of embodiments 1 to 8, wherein processing in (C) comprises
(aa) dividing the stabilized cell-containing bodily fluid sample into at least two aliquots and enriching a cell subpopulation, preferably comprising rare cells, from at least one of the provided aliquots;
(bb) providing at least one cell-containing fraction and at least one cell-depleted fraction;
(cc) further processing the cell-depleted fraction, wherein further processing the cell-depleted fraction comprises
   (i) enriching extracellular nucleic acids, optionally extracellular DNA, from the cell-depleted fraction; and/or
   (ii) enriching extracellular vesicles from the cell-depleted fraction; and
(dd) optionally enriching intracellular nucleic acids, preferably genomic DNA, from the cell-containing fraction.

12. The method according to any one of embodiments 1 to 11, further comprising (D) processing the enriched three or more biological targets for analysis.

13. The method according to embodiment 12, wherein step (C) comprises enriching target rare cells and wherein subsequent step (D) comprises analysing the enriched rare cells, optionally wherein analysing the enriched rare cells comprises analysing the enriched rare cells on a cellular level and/or by enriching intracellular nucleic acids, preferably RNA, from the enriched rare cells.

14. The method according to embodiment 13, wherein step (D) comprises detecting enriched intracellular nucleic acids, optionally wherein detection comprises amplification and/or sequencing.

15. The method according to embodiment 13 or 14, wherein the intracellular nucleic acid comprises mRNA.

16. The method according to one or more of embodiments 1 to 15, wherein step (C) comprises obtaining a cell-depleted fraction from the stabilized cell-containing bodily fluid sample and enriching extracellular nucleic acids from the obtained cell-depleted fraction.

17. The method according to embodiment 16, wherein the extracellular nucleic acids comprises or essentially consists of extracellular DNA.

18. The method according to embodiment 16 or 17, wherein the extracellular nucleic acids comprises or essentially consists of extracellular RNA.

19. The method according to one or more of embodiments 12 to 18, wherein step (D) comprises detecting one or more target molecules within extracellular nucleic acids obtained in step (C).

20. The method according to one or more of embodiments 6 to 19, wherein step (C) comprises enriching extracellular vesicles from a cell-depleted fraction obtained from the stabilized cell-containing bodily fluid sample and wherein subsequent step (D) comprises enriching RNA from the isolated extracellular vesicles.

21. The method according to one or more of embodiments 1 to 20, wherein the extracellular vesicles comprise or essentially consist of exosomes.

22. The method according to one or more of embodiments 1 to 21, comprising enriching, preferably purifying, RNA, optionally wherein RNA enrichment comprises binding RNA to a solid phase and eluting the bound RNA from the solid phase.

23. The method according to one or more of embodiments 12 to 22, wherein step (D) comprises enriching, preferably purifying, RNA from cells, preferably from enriched target rare cells, and/or from enriched extracellular vesicles.

24. The method according to embodiment 22 or 23, having one or more of the following characteristics:
(i) the enriched RNA comprises or essentially consists of mRNA;
(ii) the enriched RNA comprises miRNA or essentially consists of small RNA up to 350 nt in length, up to 300 nt in length or up to 250 nt length, which includes miRNA.

25. The method according to one or more of embodiments 12 to 24, wherein step (D) comprises detecting one or more target nucleic acid molecules within enriched, preferably purified, nucleic acids.

26. The method according to embodiment 25, wherein the at least one target nucleic acid molecule has one or more of the following characteristics:
it is a cancer-associated tumor marker;
it is a diagnostic, prognostic and/or predictive biomarker;
it is a prognostic or predictive biomarker;
it is associated with a solid cancer, optionally a metastatic cancer;
it is associated with breast cancer or prostate cancer, in particular metastatic breast and metastatic prostate cancer;
it is a positive or negative response marker;
it is a therapeutic marker; and/or
it forms part of a panel of target nucleic acid molecules, optionally wherein a panel comprises at least 5, at least 10, at least 15, at least 20, at least 25 or at least 50 target nucleic acid molecules.

27. The method according to one or more of embodiments 12 to 26, wherein step (D) comprises one or more of the following:
(i) it comprises reverse transcribing purified RNA to provide cDNA;
(ii) it comprises performing at least one amplification step;
(iii) it comprises performing a quantitative polymerase chain reaction; and/or
(iv) it comprises analyzing intact cells, optionally wherein the cells are circulating tumor cells.

28. The method according to one or more of embodiments 1 to 27, comprising enriching target rare cells and/or extracellular vesicles by affinity capture.

29. The method according to one or more of embodiments 1 to 28, wherein the cell-containing bodily fluid has one or more of the following characteristics:
it is a circulating bodily fluid;
it is selected from blood, urine, saliva, synovial fluids, amniotic fluid, lachrymal fluid, lymphatic fluid, liquor, cerebrospinal fluid, sweat, ascites, milk, bronchial lavage, peritoneal effusions and pleural effusions, bone marrow aspirates and nipple aspirates, semen/seminal fluid, body secretions or body excretions;
it is selected from blood and urine; and/or
it is blood.

30. The method according to one or more of embodiments 1 to 21, wherein the stabilization composition comprises at least one primary, secondary or tertiary amide.

31. The method according to embodiment 30, wherein the stabilizing composition comprises at least one primary, secondary or tertiary amide according to formula 1

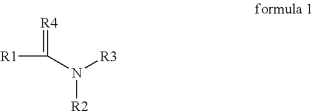

wherein R1 is a hydrogen residue or an alkyl residue, preferably a C1-C5 alkyl residue, a C1-C4 alkyl residue or a C1-C3 alkyl residue, more preferred a C1-C2 alkyl residue, R2 and R3 are identical or different and are selected from a hydrogen residue and a hydrocarbon residue, preferably an alkyl residue, with a length of the carbon chain of 1-20 atoms arranged in a linear or branched manner, and R4 is an oxygen, sulphur or selenium residue, preferably R4 is oxygen.

32. The method according to embodiment 31, wherein the at least one compound according to formula 1 is a primary, secondary or tertiary carboxylic acid amide.

33. The method according to embodiment 30 or 31, wherein the stabilizing composition comprises a N,N-dialkylpropanamide, preferably N,N-dimethlypropanamide and/or butanamide.

34. The method according to one or more of embodiments 1 to 33, wherein the stabilization composition comprises at least one poly(oxyethylene) polymer.

35. The method according to embodiment 34, wherein the poly(oxyethylene) polymer is a polyethylene glycol.

36. The method according to embodiment 34 or 35, wherein the stabilizing composition has one or more of the following characteristics:
a) the comprised poly(oxyethylene) polymer is an unsubstituted polyethylene glycol;
b) the composition comprises a poly(oxyethylene) polymer which is a high molecular weight poly(oxyethylene) polymer having a molecular weight of at least 1500;
c) the composition comprises at least one poly(oxyethylene) polymer having a molecular weight below 1500, preferably a low molecular weight poly(oxyethylene) polymer having a molecular weight of 1000 or less, optionally wherein the molecular weight lies in a range selected from 100 to 1000, 200 to 800, 200 to 600 and 200 to 500;
d) the composition comprises a poly(oxyethylene) polymer which is a high molecular weight poly(oxyethylene) polymer having a molecular weight of at least 1500 and comprises a low molecular weight poly(oxyethylene) polymer having a molecular weight of 1000 or less; and/or
e) the composition comprises a poly(oxyethylene) polymer which is a high molecular weight poly(oxyethylene) polymer and a poly(oxyethylene) polymer which is a low molecular weight poly(oxyethylene) polymer having a molecular weight of 1000 or less, wherein said high molecular weight poly(oxyethylene) polymer has a molecular weight that lies in a range selected from 1500 to 50000, 2000 to 40000, 3000 to 30000, 3000 to 25000, 3000 to 20000 and 4000 to 15000 and/or wherein said low molecular weight poly(oxyethylene) polymer has a molecular weight that lies in a range selected from 100 to 1000, 200 to 800, 200 to 600 and 200 to 500.

37. The method according to one or more of embodiments 1 to 36, wherein the stabilization composition comprises at least one apoptosis inhibitor, preferably a caspase inhibitor.

38. The method according to embodiment 37, wherein the apoptosis inhibitor wherein the caspase inhibitor has one or more of the following characteristics:
a) the caspase inhibitor is a pancaspase inhibitor;
b) the caspase inhibitor comprises a caspase-specific peptide;
c) the caspase inhibitor comprises a modified caspase-specific peptide that is modified, preferably at the carboxyl terminus, with an O-Phenoxy (OPh) group;
d) the caspase inhibitor comprises a modified caspase-specific peptide that is modified, preferably at the N-terminus, with a glutamine (Q) group;
e) the caspase inhibitor is selected from the group consisting of Q-VD-OPh, Boc-D-(OMe)-FMK and Z-Val-Ala-Asp(OMe)-FMK;
f) the caspase inhibitor is selected from the group consisting of Q-VD-OPh and Z-Val-Ala-Asp(OMe)-FMK; and/or
g) the caspase inhibitor is Q-VD-OPh.

39. The method according to one or more of embodiments 1 to 38, wherein the stabilizing composition comprises:

per variant A
(a) at least one primary, secondary or tertiary amide, preferably as defined in any one of embodiments 31 to 33, and
(b) at least one poly(oxyethylene) polymer, preferably as defined in embodiment 35 or 36, and
(c) optionally at least one apoptosis inhibitor, preferably a caspase inhibitor as defined in embodiment 38;

per variant B
(a) at least one primary, secondary or tertiary amide, preferably as defined in any one of embodiments 31 to 33,
(b) optionally at least one poly(oxyethylene) polymer, preferably as defined in embodiment 35 or 36, and
(c) at least one apoptosis inhibitor, preferably a caspase inhibitor as defined in embodiment 38;

per variant C
(a) optionally at least one primary, secondary or tertiary amide, preferably as defined in any one of embodiments 31 to 33,
(b) at least one poly(oxyethylene) polymer, preferably as defined in embodiment 35 or 36, and
(c) at least one apoptosis inhibitor, preferably a caspase inhibitor as defined in embodiment 38.

40. The method according to embodiment 39, wherein the stabilizing composition comprises:
(a) at least one primary, secondary or tertiary amide, preferably as defined in any one of embodiments 31 to 33,
(b) at least one poly(oxyethylene) polymer, preferably as defined in embodiment 35 or 36, and
(c) at least one apoptosis inhibitor, preferably a caspase inhibitor as defined in embodiment 38.

41. The method according to one or more of embodiments 1 to 40, having one or more of the following characteristics:
(i) the stabilization of the cell-containing body fluid sample does not involve the use of additives in a concentration wherein said additives would induce or promote lysis of nucleated cells;
(ii) the stabilization does not induce protein-nucleic acids or protein-protein cross-links;
(iii) the stabilization does not involve the use of a cross-linking agent that induces protein-nucleic acid and/or protein-protein crosslinks, such as formaldehyde, formaline, paraformaldehyde or a formaldehyde releaser;
(iv) the stabilization does not involve the use of toxic agents; and/or
(v) the stabilizing agents are contained in an stabilization composition comprising water.

42. The method according to one or more of embodiments 1 to 41, wherein the stabilizing composition comprises a chelating agent, optionally EDTA.

43. The method according to one or more of embodiments 1 to 42, wherein the cell-containing bodily fluid is blood and wherein the stabilizing composition comprises an anticoagulant, preferably a chelating agent.

44. The method according to one or more of embodiments 1 to 43, wherein the cell-containing bodily fluid, preferably blood, is contacted with:
a) one or more compounds according to formula 1 above;
b) at least one high molecular weight poly(oxyethylene) polymer having a molecular weight of at least 3000 and optionally at least one low molecular weight poly(oxyethylene) polymer having a molecular weight of 1000 or less;
c) at least one caspase inhibitor; and
d) optionally a chelating agent, preferably EDTA.

45. The method according to one or more of embodiments 1 to 44, wherein the cell-containing bodily fluid is blood and the blood is contacted with:
a) one or more compounds according to formula 1 above;
b) at least one high molecular weight poly(oxyethylene) polymer having a molecular weight that lies in a range of 3000 to 40000, such as in a range of 3000 to 30000 or 3500 to 25000 and at least one low molecular weight poly(oxyethylene) polymer having a molecular weight of 1000 or less, such as in a range of 100 to 800, 200 to 800 or 200 to 500;
c) at least one caspase inhibitor, preferably a pancaspase inhibitor, optionally Q-VD-OPh; and
d) an anticoagulant which preferably is a chelating agent, preferably EDTA,

wherein after the blood sample has been contacted with said additives and optionally further additives used for stabilization the resulting mixture/stabilized blood sample comprises
the one or more compounds according to formula 1 in a concentration that lies in a range of 0.3% to 4%, such as 0.5 to 3%, 0.5 to 2% or 0.75 to 1.5%,
the high molecular weight poly(oxyethylene) polymer in a concentration that lies in a range of 0.2% to 1.5% (w/v), such as 0.25% to 1.25% (w/v), 0.3% to 1% (w/v) or 0.4% to 0.75% (w/v),
the low molecular weight poly(oxyethylene) polymer in a concentration that lies in the range of 1.5% to 10%, such as 2% to 6%, and
the caspase inhibitor in a concentration that lies in a range of 1 μM to 10 μM, such as 3 μM to 7.5 μM.

46. The method according to any one of embodiments 1 to 45, wherein processing step (C) comprises subjecting the stabilized cell-containing bodily fluid sample or a cell-containing fraction obtained from the stabilized cell-containing bodily fluid sample to a density gradient centrifugation step, optionally wherein the cell-containing bodily fluid sample is blood.

47. The method according to embodiment 46, wherein the stabilized blood sample or a cell-containing fraction obtained from the stabilized blood sample is contacted with a density gradient medium.

48. The method according to embodiment 46 or 47, wherein the stabilized blood sample or a cell-containing fraction obtained from the stabilized blood sample is diluted with a dilution solution prior to performing the density gradient centrifugation step, preferably prior to contacting the diluted sample with the density gradient medium.

49. The method according to embodiment 48, wherein the stabilized blood sample or a cell-containing fraction obtained from the stabilized blood sample is diluted using a dilution solution that has one or more of the following characteristics:

(a) it is a hypotonic solution or an isotonic solution;
(b) it comprises a tonicity modifier;
(c) it comprises a polyol, optionally a sugar or sugar alcohol;
(d) it comprises a sugar, optionally glucose;
(e) it comprises a sugar alcohol, optionally glycerol; and/or
(f) it comprises a salt, optionally an alkali metal salt, optionally a chloride salt.

50. The method according to embodiment 48 or 49, wherein the dilution solution comprises a reducing sugar, optionally glucose, in a concentration that lies in a range of 2-10%, 3-7% or 4-6% (w/v).

51. The method according to any one of embodiments 48 to 50, wherein the dilution solution comprises a sugar alcohol, optionally glycerol and a salt, optionally an alkali metal salt.

52. The method according to embodiment 51, wherein the dilution solution comprises up to 0.5M glycerol and up to 2% sodium chloride, optionally wherein the dilution solution comprises 0.7-1.2% sodium chloride and 0.075-0.15M glycerol.

53. The method according to any one of embodiments 48 to 52, wherein the dilution solution achieves that after density gradient centrifugation at least 60% or at least 70% of white blood cells can be recovered from the stabilized sample, compared to an EDTA stabilized blood sample.

54. The method according to any one of embodiments 48 to 53, wherein the dilution solution is selected from
(i) 5% (w/v) glucose,
(ii) 0.9% NaCl+0.1 M glycerol, and
(iii) a dilution solution comprising at least one tonicity modifier and having a osmolality that corresponds to the osmolality of the dilution solution defined in (i) or (ii), or wherein the osmolality is within a range of +/−20%, +/−15% or +/−10% of the osmolality of the solution as defined in (i) or (ii).

55. The method according to one or more of embodiments 48 to 54, wherein the stabilized blood sample or a cell-containing fraction obtained from the stabilized blood sample is incubated no longer than 10 min, no longer than 5 min or no longer than 3 min in the dilution solution before contacting the diluted sample with the density gradient medium, wherein preferably, the diluted sample is directly processed after dilution by contacting the diluted sample with the density gradient medium.

56. The method according to one or more of embodiments 46 to 55, wherein after density gradient centrifugation, different layers are formed, wherein the formed layers comprise a PBMC layer.

57. The method according to embodiment 56, comprising collecting the formed PBMC layer thereby providing a PBMC fraction.

58. The method according to embodiment 56 or 57, comprising isolating circulating tumor cells from the collected PBMC fraction.

59. The method according to any one of embodiments 46 to 58, comprising isolating genomic DNA from the collected PBMC fraction, from which circulating tumor cells were optionally deleted in advance.

60. The method according to any one of embodiments 46 to 59, comprising washing the collected PBMC fraction using a buffer, optionally using a PBS buffer.

61. The method according to any one of embodiments 46 to 59, wherein at least a portion of the PBMC cells are subjected to white blood cell counting.

62. The method according to any one of embodiments 1 to 61, comprising obtaining a cellular fraction from the stabilized cell-containing bodily fluid sample and isolating genomic DNA from the cellular fraction, wherein the cellular fraction is stored, optionally frozen, prior to genomic DNA isolation.

63. The method according any one of the preceding embodiments, comprising enriching a cell population or individual cells using cell sorting.

64. The method according to any one of the preceding embodiments, wherein the cell-containing bodily fluid sample is blood and step (C) comprises enriching target lymphocytes as cell subpopulation from the stabilized sample.

65. The method according to embodiment 64, wherein the lymphocytes are selected from T4 and/or T8 lymphocytes.

66. The method according to embodiment 64 or 65, wherein the stabilized blood sample was obtained from a patient with immune deficiency.

67. The method according to any one of the preceding embodiments, wherein the cell-containing bodily fluid sample is blood and step (C) comprises enriching platelets as cell subpopulation form the stabilized sample, optionally wherein step (D) is performed and comprises isolating RNA from the enriched platelets.

68. The method according to any one of the preceding embodiments, wherein the cell-containing bodily fluid sample is blood and step (C) comprises enriching blast cells as cell subpopulation from the stabilized sample.

69. The method according to embodiment 68, wherein the blast cells are enriched by affinity capture, optionally using magnetic particles.

70. The method according to embodiment 68 or 69, wherein blast cells are enriched by targeting cell surface markers, optionally CD34 and/or CD117.

71. The method according to any one of embodiments 68 to 70, wherein the stabilized blood sample was obtained from a patient with acute myeloid leukemia.

72. The method according to any one of embodiments 1 to 71, wherein step (B) comprises transporting and/or storing the stabilized cell-containing bodily fluid sample prior to (C).

73. The method according to embodiment 72, wherein storing comprises transferring the stabilized cell-containing bodily fluid sample from the site of collection and stabilization to a distinct site for processing.

74. The method according to any one of embodiments 1 to 73, wherein the stabilized cell-containing bodily fluid sample is kept for up to 12 h or up to 24 h prior to processing step (C).

75. The method according to any one of embodiments 1 to 74, wherein the stabilized cell-containing bodily fluid sample is kept for up to 36 h or up to 48 h prior to processing step (C).

76. The method according to any one of embodiments 1 to 75, wherein the stabilized cell-containing bodily fluid sample is kept for up to 60 h or up to 72 h prior to processing step (C).

77. The method according to any one of embodiments 1 to 76, comprising keeping the stabilized cell-containing body fluid sample for at least 6 h, at least 8 h or at least 12 h prior to processing step (C).

78. The method according to any one of embodiments 1 to 77, comprising keeping the stabilized cell-containing body fluid sample for at least 16 h, at least 24 h or at least 48 h prior to processing step (C).

79. The method according to one or more of embodiments 1 to 79, wherein step (C) comprises isolating as biological targets at least circulating tumor cells, genomic DNA and circulating cell-free DNA.

80. The method according to embodiment 79, wherein step (D) is performed and comprises isolating RNA from the circulating tumor cells and detecting biomarker RNA molecules in the isolated RNA.

81. The method according to embodiment 81, wherein the isolated RNA is mRNA. 82. Use of a dilution solution as defined in any one of embodiments 49 to 54, for treating a stabilized blood sample or a cell-containing fraction thereof, wherein the blood sample was stabilized with a stabilization composition comprising (a) at least one primary, secondary or tertiary amide, (b) at least one poly(oxyethylene) polymer, and/or at least one apoptosis inhibitor, optionally a stabilization composition as defined in any one of embodiments 30 to 44.

83. Use according to embodiment 83, for restoring the density of comprised mononucleated cells, preferably for a gradient density centrifugation.

84. Use according to embodiment 82 or 83, wherein the dilution solution is contacted with the stabilized blood sample or a cell-containing fraction thereof prior to contacting with the gradient density medium.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification and claims, the singular forms “a”, “an” and “the” include plural aspects unless the context clearly dictates otherwise. The terms “include,” “have,” “comprise” and their variants are used synonymously and are to be construed as non-limiting. Throughout the specification, where compositions are described as comprising components or materials, it is contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

The term “enriching” “enrichment” and similar terms is used herein a broad sense and encompasses e.g. any form of enrichment such as in particular the isolation and purification of the target (e.g. nucleic acids such as DNA and/or RNA, rare cells such as circulating tumor cells, extracellular vesicles, etc. from a sample).

### EXAMPLES

The following examples demonstrate that the method according to the present disclosure has important advantages, thereby allowing to perform multimodal analyses based on a single cell-containing bodily fluid sample collected and stabilized using the stabilization technology according to the present disclosure:
1) The antigenic makeup of comprised cells stabilized with the stabilization technology of the present disclosure is preserved.
2) The stabilization technology of the present disclosure can be used in conjunction with different rare cell enrichment techniques (e.g. density gradient centrifugation, Parsortix device, AdnaTest technology, CellSearch).
3) The stabilization technology of the present disclosure can be used for analysis of cellular transcriptome (e.g. RNA content of comprised cells, such as rare cells and/or abundant cells).
4) The stabilization technology of the present disclosure can be used for analysis of circulating transcriptome (e.g. RNA from extracellular vesicles).
5) The stabilization technology of the present disclosure allows multimodality testing (e.g. the analysis of CTCs, ccfDNA and leukocyte derived genomic DNA (gDNA) from a single stabilized blood sample).

The below examples show that the stabilization technology used in the present method advantageously achieves the stabilization of spiked tumor cells and moreover preserves their core surface structures, transcriptome and genome. Immunocytochemical staining of MCF7 tumor cell line cells stabilized in PAXgene Blood ccfDNA solution (a stabilizing composition according to the present invention) demonstrated comparable results with unstabilized MCF7, indicating preservation of cellular antigenic makeup and morphology. Moreover, the cell density could be restored by adding specific solutions to the stabilized sample, such as a blood sample, thereby allowing cell separation using gradient density centrifugation. This approach enables the classical density-based separation of blood fractions, e.g. in order to enrich and thus concentrate PBMCs and CTCs in one layer.

The compatibility of collected blood stabilized with the stabilization technology of the present disclosure as front-end solution for different CTC analyzing workflows is demonstrated, based on label-independent enrichment and cellular read-out (Parsortix, ANGLE plc) and label-dependent enrichment with molecular read-out (AdnaTest ProstateCancerPanel AR-V7, QIAGEN GmbH). The results show that both approaches are compatible with the stabilization technology according to the present disclosure with high level of CTC stabilization and recovery. Moreover, enriched CTC could be advantageously used for RNA based analysis. These data provide evidence for sufficient transcriptome stabilization of cells collected into collection tubes comprising the stabilization composition according to the present disclosure. The below examples furthermore demonstrate that not only cellular RNA, but also circulating RNA (packaged in extracellular vesicles, EVs) is available for analysis. The cell-containing bodily fluid samples stabilized with the stabilization technology according to the present disclosure are thus suitable for multimodality testing of different biological targets comprised in a cell-containing bodily fluid sample such as blood. As shown exemplary based on the established workflow based on AdnaTest ProstateCancerPanel AR-V7 test, a single stabilized blood sample can be used for the analysis of CTCs, ccfDNA and leukocyte derived genomic DNA.

The performed examples are explained in the following:

### 1. Example 1: Evaluation of Antigenic Makeup Preservation on Cells Stabilized Using the Stabilization Technology According to the Present Disclosure. Immunocytochemical Staining of Untreated and Stabilized MCF7 Cancer Cell Line Cells

### Preparation of MCF7 Cytospins

Human breast cancer cell line cells (MCF7) were used as CTC model for evaluation of the impact of PAXgene Blood ccfDNA stabilizing solution (PAXccfDNA) on antigen preservation and accessibility. The PAXgene Blood ccfDNA stabilizing solution is a commercially available stabilizing composition according to the present invention which comprises the stabilizing agents (a) to (c) and an anticoagulant. It is comprised (1.5 ml) in the commercially available PAXgene Blood ccfDNA tubes (PreAnalytiX).

Cultured MCF7 cells were trypsinised, washed in PBS and incubated either in PBS or in PAXccfDNA solution for 30 min at RT. Subsequently, cytospins were prepared, dried at RT overnight and stored at +4C until being stained.

### Immunocytochemical Staining

Cells on the cytospins were fixed, permeabilized, treated against unspecific binding of antibodies (blocking step) and stained with fluorescently labeled antibodies against human pan-cytokeratin and DAPI for nuclear staining for 1 hour at RT. Subsequently cytospins were washed, covered and analysed by fluorescent microscopy within 1 week.

### Results

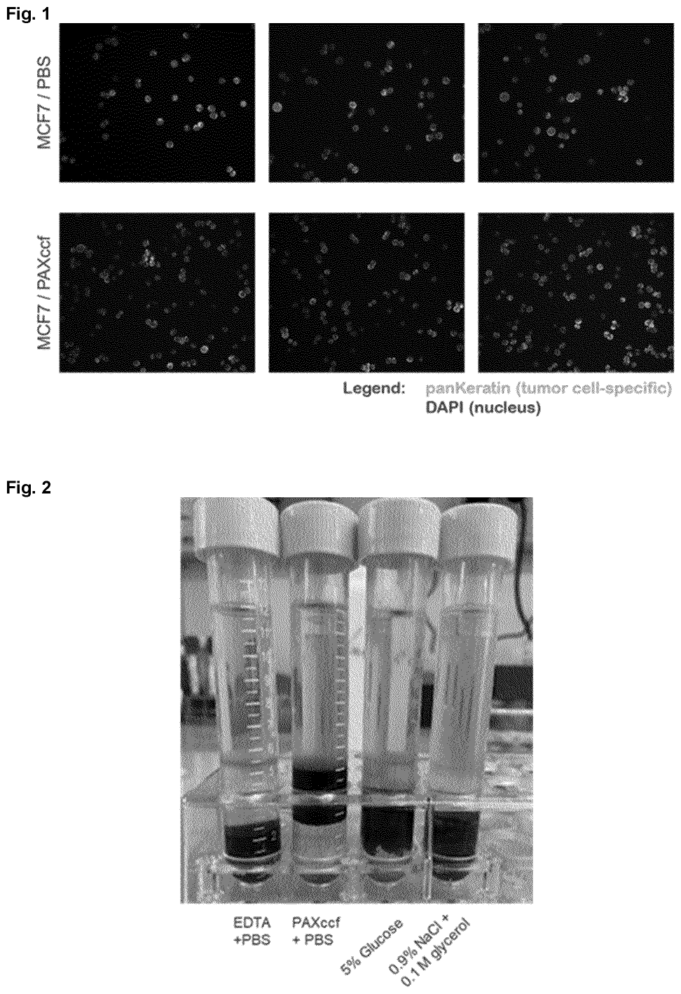

Presence of specific signal from fluorescently-labelled anti-human pan cytokeratin antibodies on unstabilized and stabilized (in the stabilization solution) cancer cells demonstrates feasibility of antigenic makeup assessment on the cells stabilized in PAXgene Blood ccfDNA tubes (FIG. 1).

Pan-Cytokeratin was well-detected on the cell-surface in the stabilized samples, demonstrating that the cell surface antigens are preserved. Nuclei staining confirmed cytosplasmatic staining of cytokeratins and preserved nuclei (i.e. morphology) of stained cells.

### 2. Example 2: Use of the Stabilization Technology for CTC Enrichment and Analysis

2.1. Combination of the Stabilization Technology with Ficoll-Density Centrifugation for CTC Enrichment

Ficoll-density centrifugation is a commonly used method for separation of blood fractions and thus cell populations into fractions based on their density. Nucleated blood cells have a density of approximately 1.062 g/ml and can be effectively separated from red blood cells (1.092 g/ml) and platelets (1.030 g/ml) when centrifuged on a ficoll layer (1.077 g/ml) or similar density gradient medium. The resulting interphase contains PBMC fraction, including CTCs and other rare nucleated cells.

It was observed that blood stabilized with the PAXgene Blood ccfDNA stabilizing solution does not form plasma/PBMC/red blood cell layers as those typically observed for EDTA-preserved blood (taken as reference), if diluted with a common PBS buffer (see FIG. 2).

Based on these observations, inter alia different slightly hypo- and isotonic dilution solutions were tested in order to restore density of the PAXccfDNA-stabilized blood cells. Isotonic 0.9% NaCl was considered as reference. Next, isoosmolar solutions containing substances able to penetrate the cell membrane (e.g. glycerol) were included into the dilution solution for testing. The aim was to obtain a typical layer formation suitable for obtaining different density based fractions that are of interest, in particular for a multimodal analysis. The efficacy was measured as number of recovered white blood cells (WBCs) after ficoll-density centrifugation.

### Processing of Blood Samples

Whole blood collected into EDTA (BD) or PAXgene Blood ccfDNA tubes (PreAnalytiX) was used. For obtaining EDTA stabilized blood, the BD Vacutainer was used (EDTA concentration in the stabilized blood is approx. 1.8 mg/ml).

4 ml of whole blood was taken, diluted with 4 ml of respective dilution solution for the indicated time (see below) and layered over 4 ml of Ficoll-Paque PLUS (GE Healthcare, density 1.077 g/ml). Samples were immediately centrifuged at 400×g for 40 min without acceleration and brake. After centrifugation the upper plasma fraction was discarded and only the PBMC ring was transferred into a new 15 ml tubes, filled up with PBS and centrifuged at 300×g for 10 min (acceleration and brake at maximum). After removal of the supernatant, the pellet was resuspended in 200 μl PBS and used for WBC counting (Beckman Coulter). Amount of WBC per ml of whole blood was calculated with consideration of 1.15 dilution factor for the PAXccfDNA tube.

### Test Settings

The following dilution solutions comprising different tonicity modifiers and concentrations were tested (Table 1):

TABLE 1
Substances and incubation times tested for density-based MNC (mononuclear cells) enrichment

| | | | | | | | | | | | NaCl | | |
|---|---|---|---|---|---|---|---|---|---|---|---|---|---|
| Solution, | | | Glucose | | Glucose | | NaCl | | NaCl | | 0.9% + 0.1M | | Glycerol |
| in PBS | PBS | | 5% | | 6.7% | | 1.0% | | 0.9% | | Glycerol | | 25% |
| Co-incubation | 0 | min; | 0 | min; | 10 | min; | 5 | min; | 0 | min; | 0 | min; | 10 min |
| time with whole | 5 | min; | 5 | min; | 15 | min | 10 | min | 5 | min | 5 | min | |
| blood, at RT | 10 | min | 10 | min | | | | | | | | | |

### Results

EDTA blood diluted with PBS without incubation was taken as reference for WBC counting.

Among the most effective dilution solutions for whole blood to obtain a classic gradient density centrifugation layer pattern essentially corresponding to EDTA-stabilized blood were: 5% glucose (glucose is taken up by blood cells) and 0.9% NaCl+0.1 M glycerol. The dilution solution comprising 0.9% NaCl+0.1 M glycerol has also a normalizing effect on shrunken cells apparently due to penetration of cell membrane by glycerol. In the final experiments very good and comparable results on WBC recovery were obtained for 5% glucose and 0.9% NaCl+0.1 M glycerol (79% and 80% from reference, respectively) added without extended incubation time (see Table 2, FIG. 2). Different dilution solutions comprising at least one tonicity modifier and having a similar osmolality as the lead dilution solutions identified in this experiment (e.g. +/−20%, +/−15% or +/−10%) may also be used and their positive effects on achieving the desired layer pattern and a WBC recovery rate of at least 50%, at least 60% and preferably at least 65% can be determined by routine experiments.

TABLE 2

| | Blood tube | | | | | |
|---|---|---|---|---|---|---|
| | | Stabilization according to the present disclosure | | | | |
| | | | | | 0.9% | 0.9% |
| | EDTA | | 5% | 5% | NaCl + 0.1M | NaCl + 0.1M |
| Buffer | PBS | PBS | Glucose | Glucose | Glycerol | Glycerol |
| Co-incubation, min | 0 | 0 | 0 | 5 | 0 | 5 |
| WBC × 10\hat{ }3 in 1 ml WB | 3150.0 | 378.4 | 2487.5 | 2020.6 | 2511.6 | 2213.8 |
| Recovery | 100% | 12% | 79% | 64% | 80% | 70% |

2.2. Combination of the Stabilization Technology with AdnaTest ProstateCancerPanel AR-V7 for CTC Detection.

AdnaTest CTC enrichment relies on immunomagnetic separation of cells, captured based on expression of target proteins on their surface. Detection of the enriched cells relies on detection of tumor cell specific transcripts. According to the manufacturer's recommendations, freshly collected EDTA blood (within 4 hours post blood draw) or blood collected into ACD-A tubes and stored at +4° C. for up to 30 hours can be used.

### (a) Materials and Methods

### Cell Culture

LNCaP95 cells were cultured in phenol red-free RPMI 1640 with 10% charcoal stripped serum and 10% penicillin/streptomycin in monolayer at 37° C. and 5% CO2.

### Blood Collection and Sample Preparation

In total blood from 21 healthy volunteers was collected upon given written informed consent into PAXgene Blood ccfDNA Tubes (PreAnalytiX, Switzerland) by venepuncture of the cubital vein and the tubes were inverted 8 times immediately after blood draw according to manufacturer instructions.

For the comparison study (see (c) below) blood was collected from healthy donors into PAXgene Blood ccfDNA Tubes and BCTs of the Provider Streck according to the manufacturer instructions.

Blood samples were pooled per donor and blood collection tube (BCT), 5 ml aliquoted into 15 ml conical tubes within 30 min upon blood draw and immediately spiked. After being manually spiked with 20 LNCaP95 or 20 μl PBS cells per sample, blood samples were stored at 2-8C or RT until being processed according to the study design.

### Enrichment and Detection of Tumor Cells Using AdnaTest ProstateCancerPanel AR-V7

AdnaTest ProstateCancerPanel AR-V7 utilizes a CTC enrichment step that is covered by the AdnaTest ProstateCancerSelect procedure. For the CTC detection, cDNA from the CTC-enriched fraction is generated.

AdnaTest ProstateCancerPanel AR-V7 relies on real-time PCR-based read-out for detection of prostate-specific PSA, PSMA, AR and AR-V7 transcripts, GAPDH as housekeeper and CD45 as leukocyte marker. Test was considered positive if at least one of the cancer specific transcripts was detected.

The AR-V7 assay includes unspecific cDNA pre-amplification step, increasing the sensitivity of the assay. Due to the pre-amplification step (18 cycles) the amplification is not linear anymore and quantification of expression of the target genes is not feasible. AR-V7 tests were performed according to the manufacturer recommendations.

### Data Evaluation

LNCaP95 cells are known to be positive for PSMA, AR and AR-V7 and have unstable expression of PSA. Therefore all tests were evaluated based on detection of PSMA, AR and AR-V7 transcripts, whereas PSA was excluded from the analyses.

Statistic evaluation of ccfDNA yield and gDNA yield was done with the use of unpaired two-tailed T-test (R-statistics version 3.5.1 using ggplot2 and ggpubr packages).

(b) Compatibility of the Stabilizing Composition According to the Present Disclosure with CTC Detection

In the first set of experiments compatibility of blood collected and stabilized with the stabilization technology according to the present disclosure with AdnaTest ProstateCancerPanel AR-V7 for detection of spiked tumor cells was evaluated. Whole blood samples from 10 donors collected into tubes comprising the stabilizing composition according to the present disclosure were pooled for each donor and aliquoted in 5 ml samples into 15 ml conical tubes. Blood samples were manually spiked with 20 LNCaP95 cells each or with 20 μl PBS as no spike control. This setup allowed to evaluate whether CTCs are detectable in the collected stabilized blood and whether stabilization reagent itself has any impact on the test performance (spiked samples and no spike control, respectively). All samples were stored at 2-8° C. until being processed 3 hrs, 24 hrs, 30 hrs, and 48 hrs after spiking.

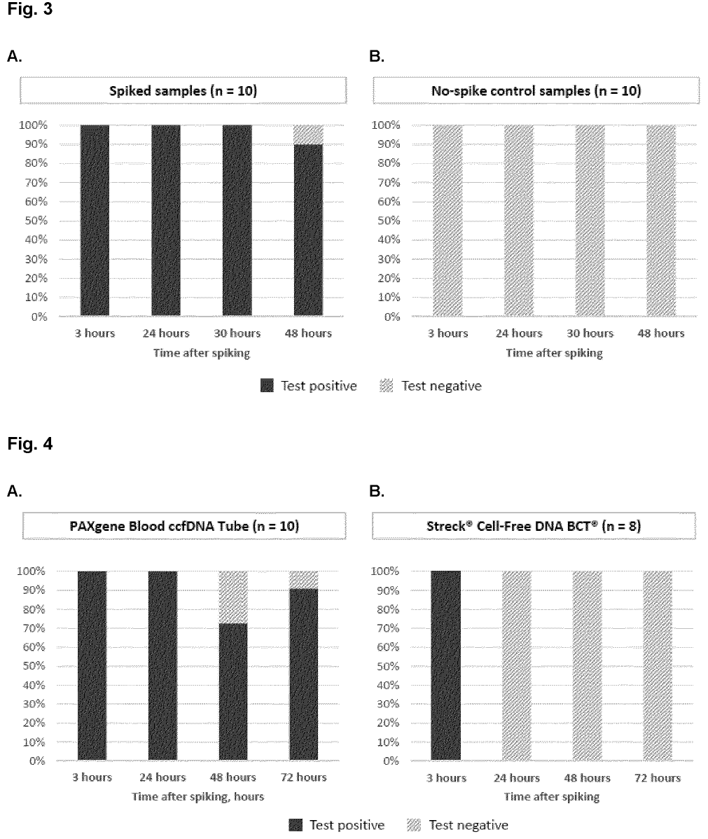

The data demonstrate positivity of the test in samples spiked with tumor cells at all experimental time points (3 hrs, 24 hrs, 30 hrs, and 48 hrs after spiking) (see FIG. 3A), whereas all no spike control tests were negative (see FIG. 3B). Thus, this established workflow demonstrates compatibility of PAXgene Blood ccfDNA Tubes comprising a stabilizing composition according to the present disclosure with AdnaTest ProstateCancerPanel AR-V7 for isolation and detection of CTCs. The stabilization solution according to the present disclosure itself does not cause any unspecific false-positive results.

Currently, the commercially available AdnaTest is recommended to be used with either EDTA or ACD-A collected blood within 4 and 30 hours after blood draw, respectively, if stored at 2-8C (14). Sensitivity of the assay is reported to be 90%. The data presented herein demonstrate 100% sensitivity within 30 hours on blood collected into tubes comprising the stabilizing composition according to the present disclosure and 90% sensitivity after 48 hours storage at 2-8 C.

### (c) Comparison of the Stabilization Technology of the Present Disclosure and Other Commercially Available Stabilization Technologies for CTC Preservation and Detection

Next efficiency of CTC detection from samples stored for up to 72 hours and collected into tubes comprising the stabilization composition according to the present disclosure (PAXgene Blood ccfDNA tubes) and Cell-Free DNA BCTs of the Supplier Streck (also intended for CTC preservation) was evaluated.

Similar to the previous experiments 20 LNCaP95 cells per 5 ml blood were used as CTC model. PAXgene Blood ccfDNA—stabilized samples (n=11) were stored at 2-8C, samples collected into Cell-Free DNA BCT (n=8)—at RT (according to the manufacturer recommendations) before being processed by AdnaTest ProstateCancerPanel AR-V7 as described above at 3 hrs, 24 hrs, 48 hrs, and 72 hrs after spiking.

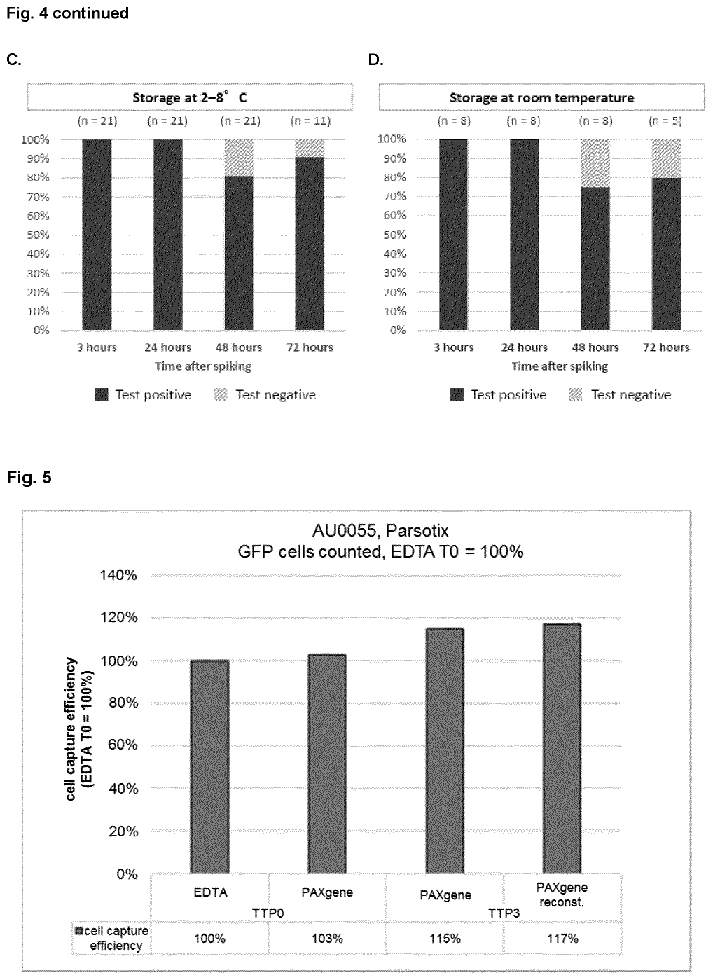

Spiked tumor cells could be efficiently detected in PAXgene Blood ccfDNA stabilized samples in 91% of cases after 72 hours storage (see FIG. 4A). In contrast, detection of spiked tumor cells was positive in blood collected into BCT of the Supplier Streck within 3 hours of storage only (see FIG. 4B). In contrast to non-crosslinking blood stabilization chemistry of PAXgene Blood ccfDNA Tubes, Cell-Free DNA BCT of the supplier Streck relies on crosslinker based cell preservation. Consequently, RNA detection is hampered. The obtained data is in line with observations made on these BCTs by others (see CTC-mRNA (AR-V7) Analysis from Blood Samples-Impact of Blood Collection Tube and Storage Time. Luk et al, Int J Mol Sci. 2017 May 12; 18(5).).

Further experiments moreover demonstrated that CTCs could also be enriched after storage at room temperature (see FIGS. 4C and 4D).

2.3 Compatibility Testing of PAXgene Blood ccfDNA Tube with Parsortix Device for CTC Enrichment in Context of all-from-One Solution.

### Study Design

The general compatibility of PAXgene ccfDNA stabilized blood with the Parsortix (Angle plc, Guildford, UK) enrichment instrument and the capture efficiency of spiked cells from (un-) stabilized blood was tested in this experiment. The Parsortix technology enriches larger and less deformable cells (e.g. CTCs) from the blood cellular components by capturing the cells in a disposable microscope-sized cassette. The cells can be stained and counted in the cassette and harvested using a reverse flow system.

In this experiment, a model system approach for CTC enrichment was used. Blood was collected from one healthy donor in EDTA and PAXgene Blood ccfDNA tubes. The blood was first either aliquoted into 5 ml (EDTA) or 6 ml (PAXgene) samples to consider the additional liquid in the PAXgene ccfDNA tube (the comprised stabilizing solution). Then all samples were spiked with 2000 cells that stably express a green fluorescent protein (purchased as MCF7-GFP cells). The advantage of this cell line is that captured cells can be detected and counted under a fluorescence microscope within the enrichment cassette without further staining or treatment.

EDTA and PAXgene stabilized blood samples were processed with the Parsortix instrument at day of collection (TTP0) and the number of GFP cells trapped in the cassette was counted. EDTA blood served as reference since capturing CTCs from unstored EDTA blood is the recommended workflow by the instrument provider and still the main sample quality used in clinical research.

After three days of blood storage at room temperature the cells were enriched either from PAXgene-stabilized whole blood (PAXgene) or whole blood was centrifuged once (15 min, 1900×g), plasma was discarded and blood was reconstituted with 3 ml PBS to recover viscosity (PAXgene reconst) before Parsortix processing. The number of GFP cells captured in the cassette was again counted using a fluorescence microscope.

### Results

At day of collection, the number of cells captured and counted was similar in blood collected in a PAXgene ccfDNA tube to the EDTA control (103% for PAXgene). After three days of storage, a comparable although slightly higher number of cells could be captured and counted in the cassette, independent of a centrifugation step before the blood processing (see FIG. 5).

### Conclusions

Blood collected in a PAXgene Blood ccfDNA tube is compatible with the Parsortix cell enrichment workflow and can be processed even after three days of storage at room temperature and plasma separation.

An all-from-one-solution to both obtain ccfDNA as well as CTCs from a blood sample collected and stabilized using the stabilization technology according to the present disclosure is therefore advantageously feasible.

### 3. Example 3: PAXgene Blood ccfDNA Tubes can be Used for Analysis of Cellular Transcriptome (RNA Content of the Cells)

Proof-of-principle experiments for CTC enrichment and RNA analysis is provided in section 2.2. AdnaTests rely on RNA based CTC detection using RT-PCR. The successful detection of spiked tumor cells as demonstrated in section 2.2. above demonstrates, that RNA content of individual cells is preserved for at least 72 hours if blood was collected into PAXgene Blood ccfDNA Tubes.

### 4. Example 4: PAXgene Blood ccfDNA Tubes can be Used for Analysis of Circulating Transcriptome (RNA from Extracellular Vesicles)

### Study Design

Compatibility of blood stabilized with the stabilization technology of the present disclosure with subsequent EV analysis was demonstrated in the following study. PAXgene Blood ccfDNA tubes were again used for blood stabilization.

Whole blood from 4 healthy donors was collected into three different blood collection tubes each, a 10 ml K2-spray dried EDTA tube (BD Vacutainer), a 10 ml Streck cfDNA BCT and a 10 ml PAXgene Blood ccfDNA tube.

From each tube 5 ml blood was processed after collection. Plasma was generated by double centrifugation and filtrated with 0.8 μm filter. RNA was isolated according to exoRNeasy Serum/Plasma Maxi Kit (QIAGEN) and eluted with 20 μl water.

Purified RNA was analysed with RT-qPCR beta-actin assay for amplification of a 294 bp fragment. The analyses was performed with a Quantitect Primer/Probe RT PCR Master Mix and 2 μl eluate.

### Quantitative, Real Time PCR Assay for Determination of Relative Difference on Beta-Actin Copies

To measure the amount of ccfDNA a real time PCR assay on RGQ (QIAGEN) was performed with 2 μl of eluate on a Rotor-Gene Q instrument (Table 3). In a 20 μl assay volume using QuantiTect Multiplex PCR Kit reagents (QIAGEN GmbH) a 294 bp fragment of the human beta-actin gene is amplified.

TABLE 3
Primers’ and probe’s sequences for the beta actin assay

| | amplicon | | |
|---|---|---|---|
| | size | Primer/Probes | |
| target | [bp] | position | sequence 5-3' |
| βactin | 294 | forward | TCA CCC ACA CTG TGC CCA TCT ACG A |
| | | reverse | CAG CGG AAC CGC TCA TTG CCA ATG G |
| | | Probe | FAM-ATG CCC TCC CCC ATG CCA TCC TGC GT- |
| | | | BHQ |

### Results

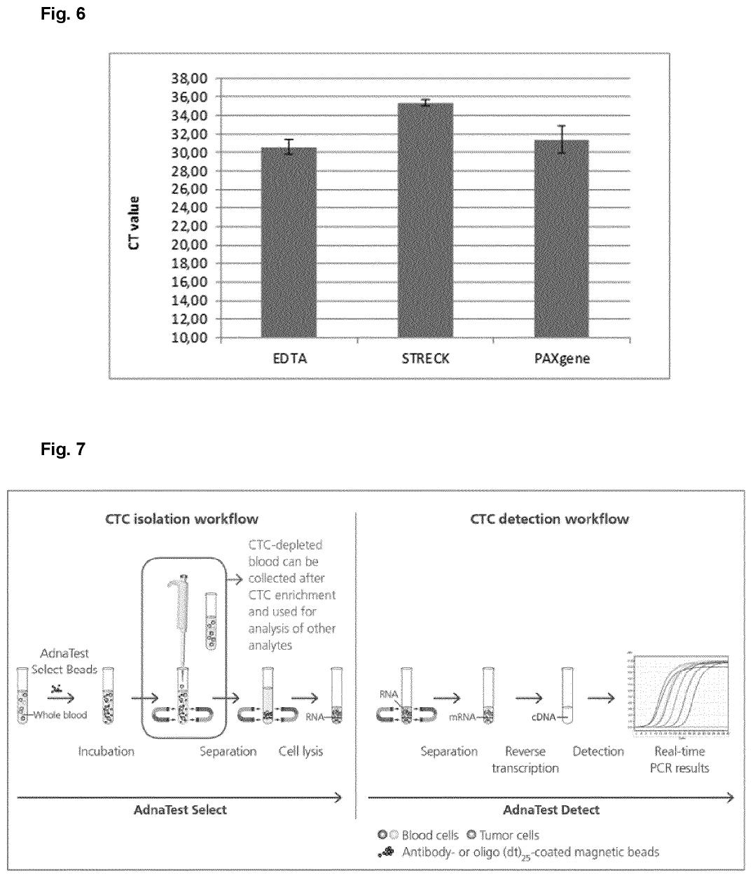

Extracellular vesicles (EVs) can be enriched from plasma generated from whole blood collected into blood collection tubes containing a stabilization composition according to the present disclosure. RNA obtained from the purified EVs could be analysed by RT-qPCR without inhibition (see FIG. 6).

In contrast, analysis of RNA isolated from EVs from whole blood collected into Streck cfDNA BCT led to increased Ct values and thus disadvantageous results, most likely because of inhibition of RT-qPCR due to crosslinks on the RNA molecules that are induced by the formaldehyde releaser based stabilization technology.

### 5. Example 5: Samples Stabilized in PAXgene Blood ccfDNA Tubes can be Used for Multimodality Testing

The examples herein demonstrate that multimodality testing of different biological targets comprises in a stabilized bodily fluid sample is feasible, as subsequently further demonstrated by way of example using a 3 from 1 workflow for the analysis of (1) CTCs, (2) ccfDNA and (3) leukocyte derived genomic DNA (gDNA) obtained from a single blood sample that was collected and stabilized with the stabilization technology according to the present disclosure.

AdnaTest Select procedure enables collection of whole blood residues after retrieval of bead bound CTCs (CTC depleted blood) (see FIG. 7). Accordingly, CTC depleted blood from all above mentioned experiments was collected in order to demonstrate feasibility of multimodality testing on blood collected into PAXgene Blood ccfDNA Tubes. PAXgene Blood ccfDNA Tubes allow for simultaneous ccfDNA and leukocyte gDNA analyses. It is subsequently demonstrated that CTC depleted blood from the experiments listed in section 2.2. can be used for ccfDNA isolation and that the yields were advantageously not affected by CTC depletion. Control samples collected in parallel from respective donors, aliquoted in 5 ml samples, spiked with 20 LNCaP95 cells and stored for the same time at 2-8 C, but not used for CTC enrichment were used as reference for ccfDNA and gDNA yield.

CTC depleted blood samples together with respective control samples were centrifuged at 1900×g for 15 min. Resulting blood fractions (plasma and cellular fraction) were used for ccfDNA extraction (after second centrifugation at 1900×g for 10 min) and gDNA isolation, respectively.

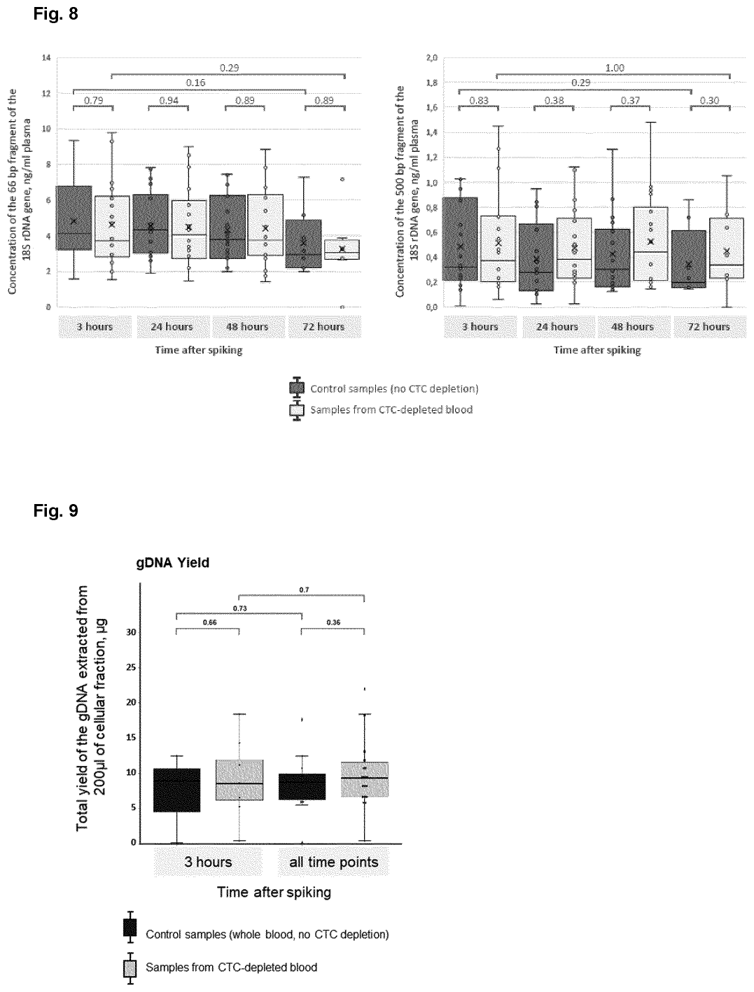

CcfDNA yield from CTC-depleted blood samples and blood used for plasma generation alone are presented in Table 4. Statistical analysis did not reveal any significant differences in ccfDNA yield neither between the arms, nor between the first and the last test time points within the same experimental arm (see FIG. 8). Thus, CTC depletion did not have a significant impact on ccfDNA yield in terms of yield and in situ stability.

TABLE 4
ccfDNA yield determined as concentration (in ng) of 66 bp and 500 bp fragments
of 18S rDNA gene, normalized to 1 ml of the utilized plasma.

| | | | | |
|---|---|---|---|---|
| Test time points | | | | |
| after spiking | 0 hrs | 24 hrs | 48 hrs | 72 hrs |

Concentration of the 66 bp fragment of 18S rDNA gene, ng/1 ml plasma

| | | | | |
|---|---|---|---|---|
| Plasma from | 4.60 ± 2.33 | 4.50 ± 2.18 | 4.41 ± 2.17 | 3.70 ± 1.46 |
| CTC-depleted | | | | |
| samples | | | | |
| Plasma generated | 4.81 ± 2.15 | 4.55 ± 1.79 | 4.31 ± 1.80 | 3.58 ± 1.72 |
| from whole blood | | | | |

Concentration of the 500 bp fragment of 18S rDNA gene, ng/1 ml plasma

| | | | | |
|---|---|---|---|---|
| Plasma from | 0.51 ± 0.40 | 0.47 ± 0.31 | 0.53 ± 0.36 | 0.51 ± 0.29 |
| CTC-depleted | | | | |
| samples | | | | |
| Plasma generated | 0.48 ± 0.34 | 0.38 ± 0.29 | 0.42 ± 0.30 | 0.34 ± 0.26 |
| from whole blood | | | | |

Similar, yield of gDNA extracted from cellular fraction obtained after centrifugation of the CTC depleted blood samples (n=8) was in range of the values reported for blood stabilized in PAXgene ccfDNA Tubes. On average 10.3 μg gDNA could be isolated from 200 μl of cellular fraction from CTC depleted samples (range 5.31-21.97 μg) in comparison to 9.43 μg gDNA from samples without CTC depletion (range 7.66-11.23 μg). There was no statistically significant difference between yield of gDNA extracted from CTC-depleted and blood used for plasma generation alone neither 3 hours after spiking and processing nor in total (all time points 3-72 hrs) (see FIG. 9). Purity of the extracted gDNA was 1.86±0.05 and 1.85±0.06 for the CTC-depleted and control samples (i.fe. generated from whole blood), respectively (average at all time points), which is in range of expected values (1.7-1.9).

### Materials and Methods

### Generation of Plasma and Cellular Fraction

Plasma from PAXgene Blood ccfDNA Tubes was generated according to the manufactures instructions. In brief, blood was centrifuged at 1900×g for 15 min. The cellular fraction and the plasma fraction were separated. The plasma containing fraction was further centrifuged at 1900×g for 10 min, plasma was collected without disturbing the respective pellet and stored at −20° C. The cellular fraction obtained after the first spin was frozen immediately at −20 C until being processed for gDNA extraction.

ccfDNA Workflow
Automated Purification of ccfDNA on the QIAsymphony

CcfDNA from 1.6-2.0 ml PAXgene plasma was isolated with the magnetic bead based extraction protocol using the QIAsymphony PAXgene Blood ccfDNA Kit (both PreAnalytiX) on the QIAsymphony instrument (QIAGEN).

### Quantitative, Real Time PCR Assay for Determination of Absolute Difference on 18S Ribosomal DNA Copies

Absolute quantification of 66 and 500 bp fragments of human 18S rDNA gene was done with the use of standard curves in ccfDNA samples from CTC-depleted and unspiked blood samples (see FIG. 8 and the workflow illustrated in FIG. 11). Real time PCR assay was performed with 8 μl of eluate in a 20 μl assay volume using QuantiTect Multiplex PCR Kit reagents (QIAGEN) on ABI 7900HT Fast Real-Time PCR-System (ThermoFisher). Calculated amounts of the 66 bp and 500 bp fragments were normalized to the volume of used plasma.

gDNA Workflow
Automated Purification of gDNA on the QIAsymphony

Genomic DNA from 200 μl of the separated cellular fraction obtained after plasma separation was isolated with the magnetic bead based extraction protocol using the QIASymphony DSP DNA Mini Kit on the QIAsymphony instrument (QIAGEN). Elution volume was 200 μl per sample.

Quantification of gDNA and Evaluation of gDNA Purity Absorbance of the gDNA was measured on NanoDrop8000 (Thermo Scientific). Absorbance was measured at 260 nm, 280 nm and 320 nm. Concentration of gDNA (μg/ml) was calculated as “50×(A260-A320)” and total amount—as concentration multiplied by the volume of the sample. Purity of the extracted gDNA was calculated as ratio of the corrected absorbance at 260 nm to corrected absorbance at 280 nm, i.e. (A260-A320)/(A280-A320). Pure DNA is characterized by A260/A280 ration of 1.7-1.9.

### Overall Conclusions—Examples 1 to 5

Cells, including CTCs and other rare cells degrade rapidly in unstabilized blood. The stabilization technology used in the method of the present disclosure (here demonstrated based on the PAXgene Blood ccfDNA Tube) allows for effective stabilization and analysis of ccfDNA levels, CTCs and extracellular vesicles, thereby enabling the parallel analysis of multiple different biological targets that can be enriched from the stabilized sample. As demonstrated herein, the stabilization technology according to the present disclosure allows to stabilize cellular antigenic makeup, genomic and transcriptomic levels as well as circulating transcriptome.

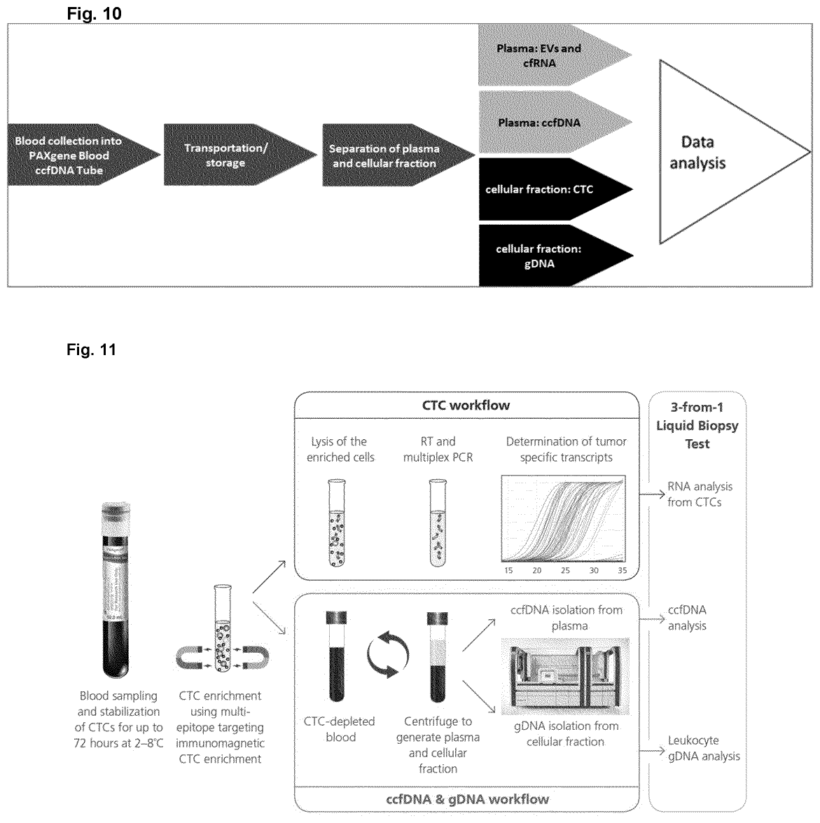

The workflow according to the present invention is thus suitable for analysis of individual liquid biopsy analytes (such as CTCs and other rare cells, ccfDNA, ctDNA, EVs, leukocyte derived gDNA, cell subpopulations) and a combination of such analytes from the same blood sample, collected into a single collection tube comprising the stabilizing composition according to the present invention (see FIG. 10). An illustrative workflow is also shown in FIG. 11.

According to one embodiment, the blood sample based workflow according to the present disclosure comprises:
blood collection in a collection tube comprising a stabilizing composition according to the present disclosure (e.g. PAXgene Blood ccfDNA tube, blood draw volume e.g. at least 5 ml, e.g. 10 ml; volume including stabilizing solution e.g. 11.5 ml), transportation into a laboratory.
A portion of stabilized blood is used for CTC enrichment (e.g. 5 ml). Untreated blood (e.g. 6.5 ml) and residual blood after CTC enrichment (approx. 4.5 ml) may be used for plasma generation (cell-depleted fraction). Plasma generation may be performed using a 2 step centrifugation protocol.
   A cellular fraction, e.g. obtained after first centrifugation is used for total gDNA extraction from PBMCs or FACS-sorting for DNA extraction from a target PBMC subpopulation.
   The generated plasma is further centrifuged in the second centrifugation step. The obtained plasma may be further aliquoted for ccfDNA and/or EV isolation.
The enriched CTCs may be further processed. E.g. the enriched CTCs may be lysed and intracellular nucleic acids (e.g. RNA, in particular mRNA) may be isolated therefrom for analysis (e.g. detection of CTC transcripts). Furthermore, intracellular nucleic acids obtained from the enriched CTCs may be sequenced.

According to one embodiment, the blood sample based workflow according to the present disclosure comprises:
blood collection in a collection tube comprising a stabilizing composition according to the present disclosure (e.g. PAXgene Blood ccfDNA tube, blood draw volume e.g. at least 5 ml, e.g. 10 ml; volume including stabilizing solution e.g. 11.5 ml), transportation into a laboratory.
separation of the stabilized blood sample into a plasma and cellular fraction by centrifugation (e.g. using a 2 step centrifugation protocol).
   An aliquot of the obtained plasma is used for direct purification of ccfDNA. A further aliquot of plasma is used for concentration of EV and subsequent isolation of RNA from the EVs.
One aliquot of the cellular fraction may be used for isolation of gDNA. Alternatively or additionally, an aliquot (preferably the majority) of the cellular fraction is used to capture CTCs and for subsequent gDNA isolation from residual PBMCs from which CTCs were depleted.
Again, the enriched CTCs may be processed further as described above.

### 6. Example 6: Further Uses of the Stabilization Technology for CTC Enrichment and Analysis According to the Invention

6.1. Further Experiments Regarding the Combination of the Stabilization Technology with Ficoll-Density Centrifugation for CTC Enrichment

The Ficoll-density centrifugation has been described above in conjunction with Example 2 and it is referred thereto for conciseness. Using the same methodology as previously described, further experiments were conducted aiming at optimization of mononuclear cells (MNCs) enrichment from blood collected and stored in PAXgene Blood ccfDNA Tubes. Resulting interphase contains PBMC fraction, including CTCs and other rare nucleated cells.

In Example 2 it was observed, that PAXccfDNA-stabilized blood does not form plasma/PBMC/red blood cell layers as those typically observed for EDTA-preserved blood (taken as reference). Hence, in relative comparison of MNC recovery to EDTA samples, PAX-stored blood samples often demonstrated only 75% of MNC recovery achievable for EDTA samples (FIG. 12).

In order to improve MNC recovery when processing samples stabilized with the technology of the invention, further and also different solvents aiming at restoring of cellular density, were evaluated.

### Results

In addition to Example 2, further concentrations were tested as well as other supplements. Comparison was done to PAX samples diluted with PBS only. The results are present in Table 5 below.

TABLE 5
Results of the MNC recovery in comparison to PAX + PBS
for different supplements (all diluted with PBS) - representation
of MNC recovery rates (% relative to PBS + EDTA).

| | | Average MNC |
|---|---|---|
| | Incubation | recovery |
| Solution | time, min | rate, % |
| 3% Glucose | 0 | 92 |
| 5% Glucose | 0 | >150 |
| 5% Glucose | 5 | >150 |
| 0.8% NaCl | 0 | >150 |
| 0.8% NaCl + 0.1M Glycerol | 0 | >150 |
| 0.9% NaCl | 0 | 126 |
| 0.9% NaCl | 5 | 91 |
| 1.0% NaCl | 5 | 95 |
| 1% DMSO | 0 | 102 |
| 2% DMSO | 0 | 94 |
| 3% DMSO | 0 | 108 |
| 0.9% NaCl + 0.1M Glycerol | 0 | 109 |
| 1.0% NaCl + 0.1M Glycerol | 0 | >150 |
| 1.0% NaCl + 0.1M Glycerol | 5 | >150 |
| 1.0% NaCl + 0.15M Glycerol | 0 | 123 |
| 1.1% NaCl + 0.15M Glycerol | 0 | 111 |

Based on these observations, different hyper- and isotonic solutions were tested in order to restore density of the PAXccfDNA-stabilized blood cells. Sufficient and outperforming recovery rates were observed for tested solutions, demonstrating success of the approach.

Different dilution solutions having a similar osmolality as the lead dilution solutions identified in Table 5 (e.g. +/−20%, +/−15% or +/−10%) may also be used and their positive effects on achieving the desired layer pattern and a WBC recovery rate of at least 50%, at least 60% and preferably at least 65% can be determined by routine experiments.

6.2. Further Experiments Concerning the Combination of PAXgene Blood ccfDNA Tube with AdnaTest ProstateCancerPanels for CTC Detection.

The AdnaTest CTC enrichment and the associated materials and methods have been described above in conjunction with Example 2 and it is here referred thereto for conciseness. Detection of the enriched cells relies on detection of tumor cell specific transcripts. According to the manufacturer's recommendations, freshly collected EDTA blood (within 4 hours post blood draw) or blood collected into ACD-A tubes and stored at +4° C. for up to 30 hours can be used.

In multiple experiments it was evaluated whether blood collected into and stored in PAXgene Blood ccfDNA Tube is compatible with the three different AdnaTests and to which extent: time of blood storage, storage conditions (room temperature, RT vs 2-8 C) and what LOD (20 tumor cells/5 ml blood vs 5 cells/5 ml blood).

A. Combination of the PACgene Blood ccfDNA Tubes with the AdnaTest ProstateCancerPanel AR-V7

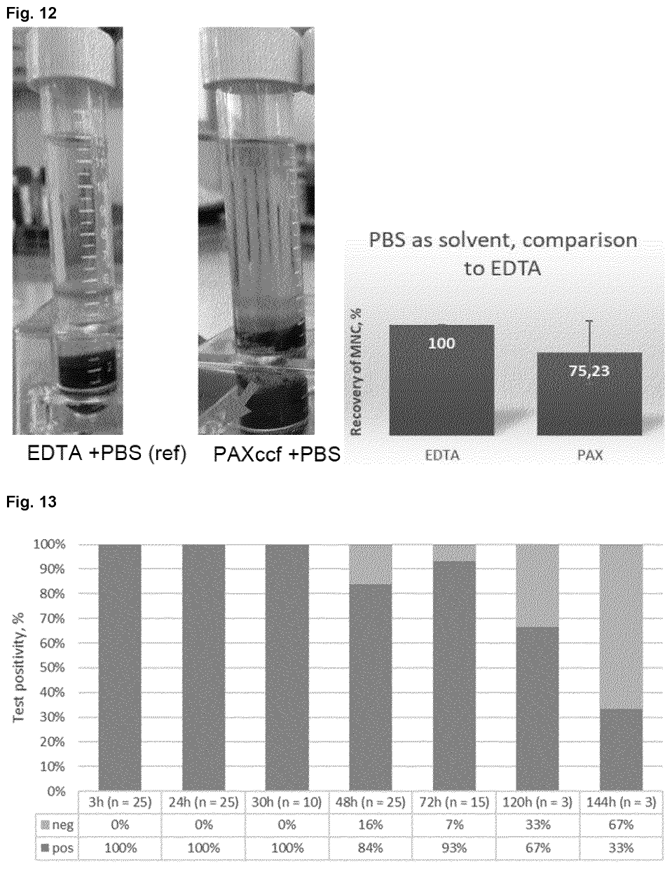

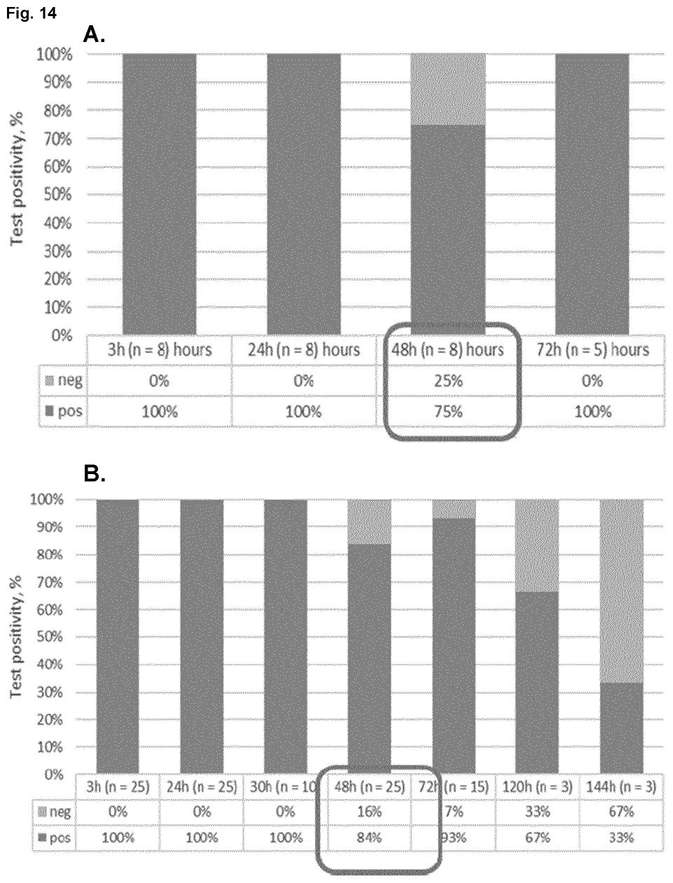

In this set of experiments, the above found compatibility of the blood collected and stabilized with the stabilizing technology according to the present disclosure with the AdnaTest ProstateCancerPanel AR-V7 for detection of spiked tumor cells was further evaluated and confirmed. Hence, in multiple experiments using the Adnatest ProstateCancerPanel AR-V7 it was shown that tumor cell detection rate in mock samples (20 LNCaP95 cells/5 ml blood) was 100% within 30 h storage at 2-8 C and decreased to 93% after 72 h (see FIG. 13). Even after 120 hrs 67% were still detected. Again, this confirms that the stabilization solution according to the present disclosure itself does not cause any unspecific false-positive results and therefore can be well integrated into the workflow described herein.

When performance of the test was evaluated in regard to storage conditions (RT vs 2-8 C), a slight decrease in test performance was observed (75% test positivity for RT-stored samples vs 84% for samples stored at 2-8 C) (see FIGS. 14A and 14B). However, overall CTCs could also be enriched after storage at room temperature.

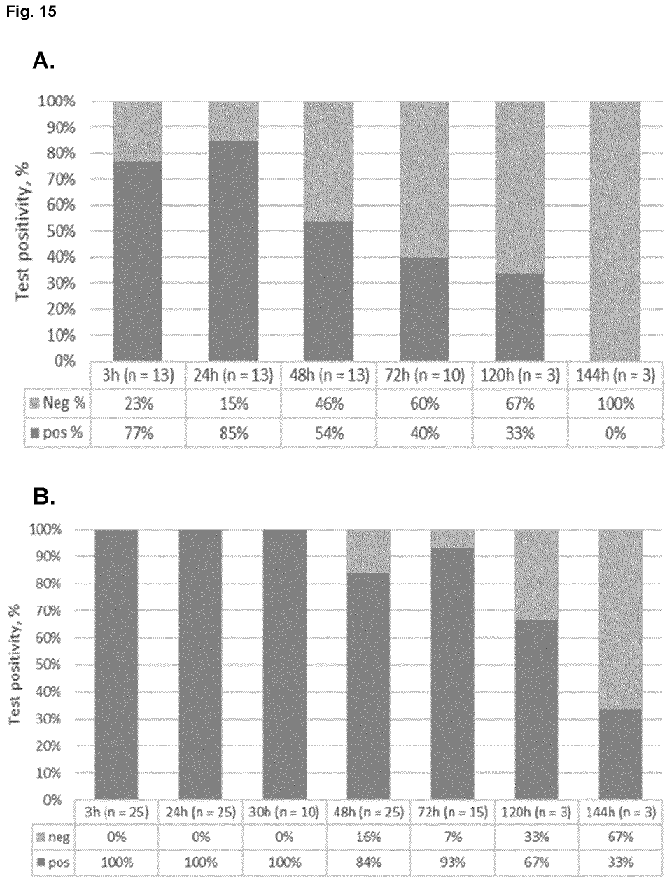

Next, limit of detection (LOD) of the test was evaluated. Samples collected into PAX ccfDNA Tubes were spiked with either 5 or 20 cells/5 ml blood. The results show that the samples spiked with 20 cell/5 ml blood (see FIG. 15B) were better detected indicating that 5 cells/5 ml blood (see FIG. 15A) are sufficient only for shorter storage times. Preferably higher cell numbers such as 20 cells/5 ml are used achieving a high sensitivity of (>90%) of the whole workflow (see FIGS. 15A and 15B).

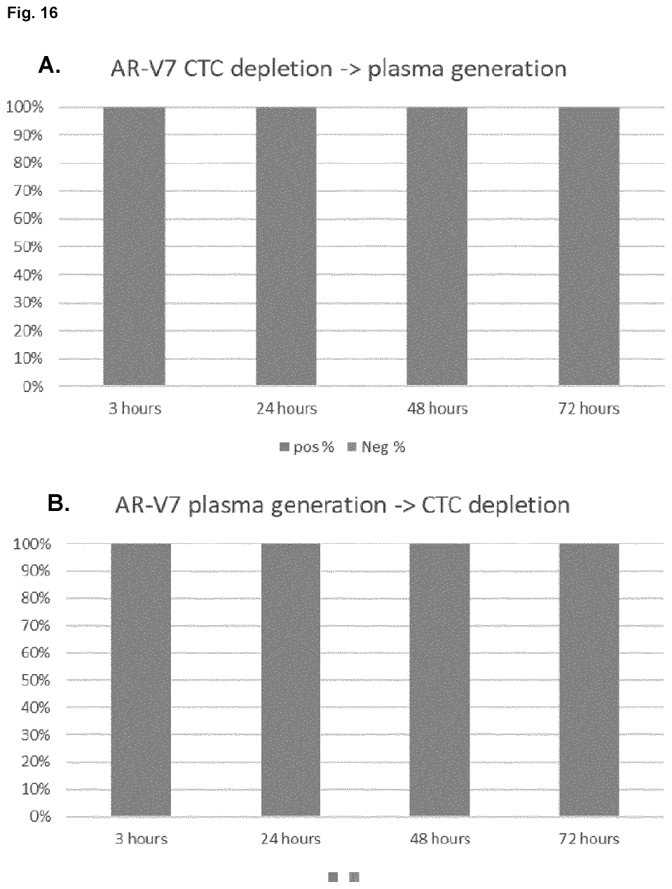

Finally, different regimens of plasma generation were tested. In the workflow used throughout the examples of the present invention blood samples were first used for CTC enrichment and CTC-depleted blood was used for plasma generation for further multimodality testing (see FIG. 16A). In an alternative plasma generation method, plasma was generated as the first step (at 1900 g for 15 min) and the cellular fraction was then reconstituted with PBS up to the initial volume and used for CTC enrichment (see FIG. 16B). The results of the detected tumor cells are shown in FIG. 16. In particular, in both plasma generation methods 100% of the spiked tumor cells were detected for storage time point up to 72 h, demonstrating that the sample stabilized by the method according to the present invention can be used for both types of plasma generation methods without negatively affecting CTC enrichment and detection.

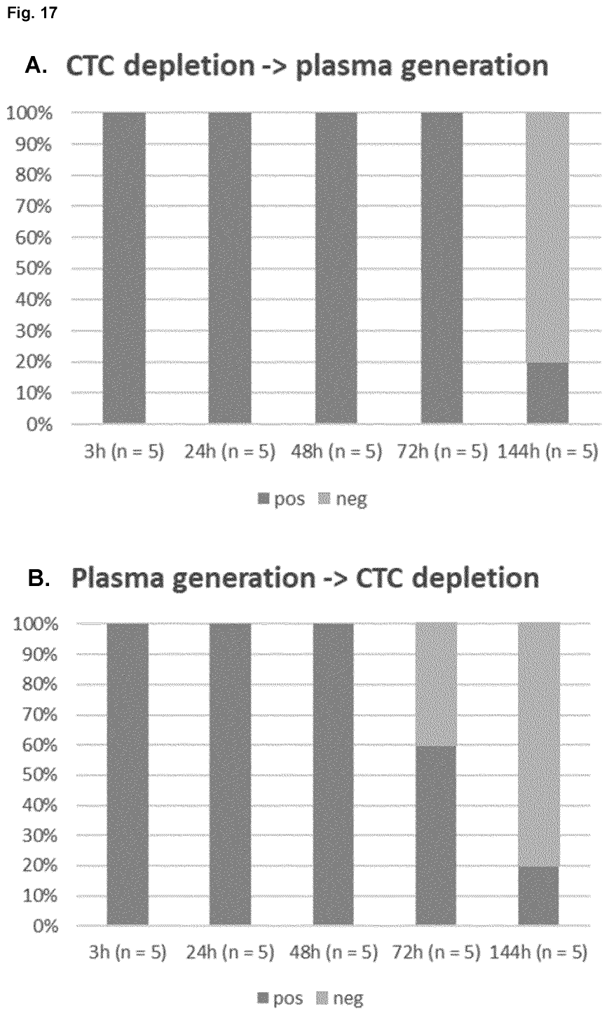

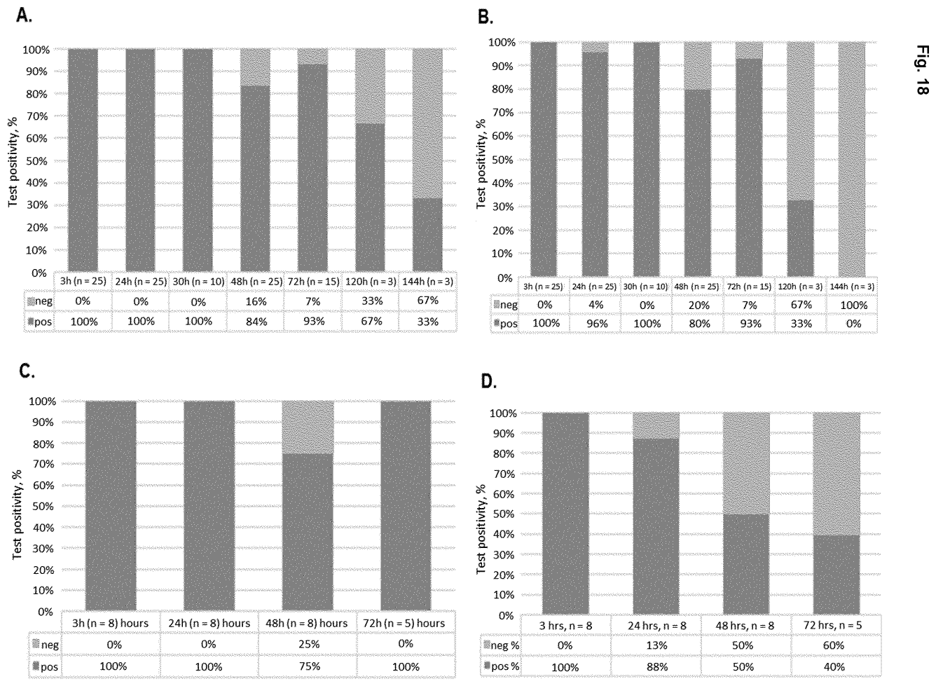

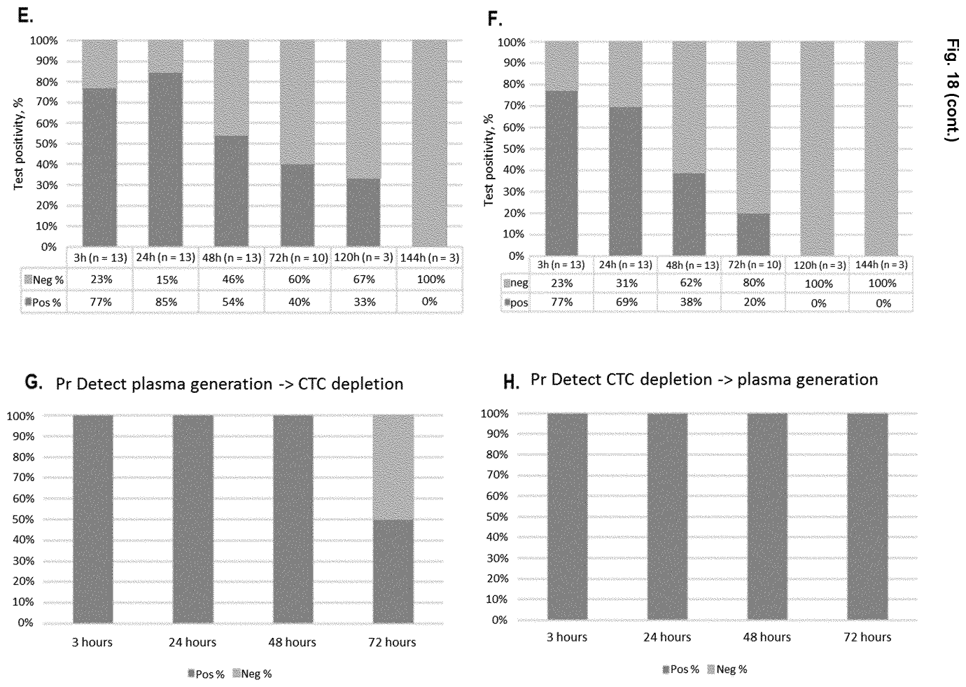

In line, similar results were observed when the same experiment regarding the plasma generation method comparison was conducted on EZ1 instrument (automated solution) with a prototype AdnaTest for EZ1 (see FIGS. 17A and 17B).

The results of the present Example indicate that either way of plasma generation (i.e. multimodality usage) is applicable.

B. Combination of the PAXgene Blood ccfDNA Tubes with the AdnaTest ProstateCancer

In this example the AdnaTest ProstateCancer (also referred to as “ProstateDirect”) is compared to the AdnaTest ProstateCancerPanel AR-V7. The AdnaTest ProstateCancer is a less sensitive test than the AdnaTest ProstateCancerPanel AR-V7 and relies on end-point PCR evaluation (whereas AR-V7 test is an RT-PCR test).

In this comparison, samples utilized in experiments described above were used for AdnaTest ProstateCancer evaluation too. It is therefore referred to the respective section above for conciseness.

The results of the comparison are shown in FIG. 18 and confirm the findings made with the AdnaTest ProstateCancer Panel AR-V7. In particular, following results were obtained:
It was shown that tumor cell detection rate in mock samples (20 LNCaP95 cells/5 ml blood) was 100% within 30 h storage at 2-8 C and decreased to 93% after 72 h (see FIG. 18A for the AdnaTest ProstateCancerPanel AR-V7 and FIG. 18B for the AdnaTest ProstateCancer).
When performance of the test was evaluated in regard to storage conditions (RT vs 2-8° C.), a slight decrease in test performance was observed (AdnaTest ProstateCancerPanel AR-V7: 75% test positivity for RT-stored samples vs 84% for samples stored at 2-8 C; AdnaTest ProstateCancer 50% test positivity for RT-stored samples vs 80% for samples stored at 2-8 C; see FIGS. 18C and 18D, respectively). Also here, overall CTCs could also be enriched after storage at room temperature.
As above, the limit of detection (LOD) was evaluated by spiking either with 5 cells/5 ml blood and testing with the AdnaTest ProstateCancerPanel (see FIG. 18E) or AdnaTest ProstateCancer (see FIG. 18F). The results confirm that the samples spiked with 20 cell/5 ml blood (see above) led to better detection indicating that 5 cells/5 ml blood are sufficient only for very short storage times. Preferably higher cell numbers such as 20 cells/5 ml are detected for both tests.
Finally, a different plasma generation method was tested. In particular, the alternative plasma generation method was used, wherein plasma was generated as the first step and the cellular fraction was used for CTC enrichment. The enriched CTC fraction was used for the AdnaTest ProstateCancerPanel AR-V7 (see FIG. 18G) or the AdnaTest ProstateCancer (see FIG. 18H). The alternative plasma generation method allowed for detection of 100% of the spiked tumor cells for storage time point up to 48 h, demonstrating that the sample stabilized by the method according to the present invention can be used for both types of plasma generation methods without negatively affecting CTC enrichment and detection. Therefore, an advantageous workflow can be provided.
   6.3. Combination of the PAXgene Blood ccfDNA Tubes with the AdnaTest ColonCancer

Performance of the AdnaTest ColonCancer was tested on a similar spike-in system as discussed above in conjunction with the AdnaTest ProstateCancer and the AdnaTest ProstateCancerPanel AR-V7. In particular, 20 T84 cells were spiked per 5 ml healthy donor blood. Samples were stored at 2-8 C using the PAXgene Blood ccfDNA Tubes compared to the test performance with samples collected into ACD-A BCTs and similarly spiked. The performance was tested at time points of 3 h, 24 h, 48 h, and 72 h after spiking.

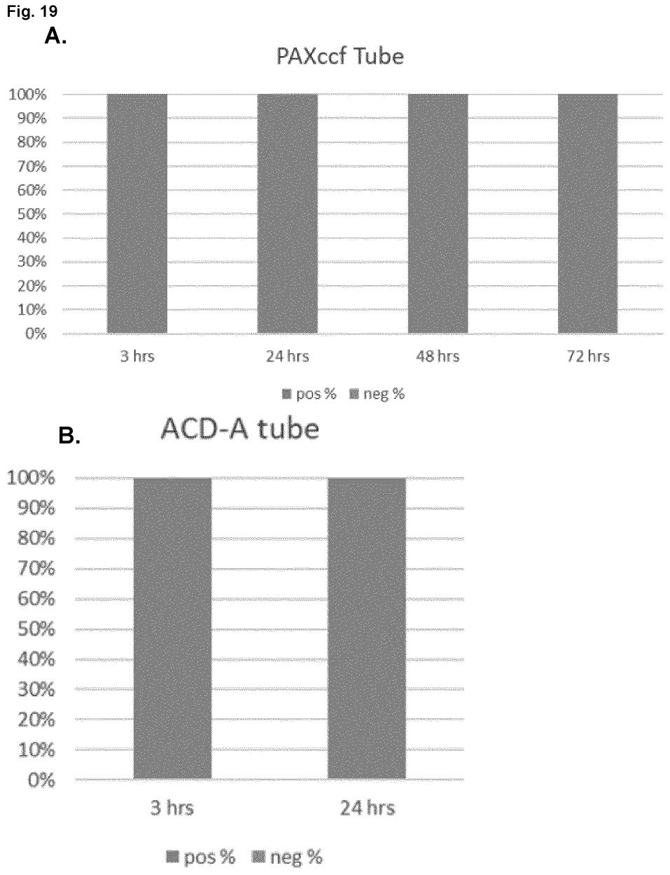

The results show that the PAXgene Blood ccfDNA Tubes that are preferably used in the workflow described herein are compatible with the AdnaTest ColonCancer and allow for detection of tumor cells upon storage of samples within 72 h (100% sensitivity) (see FIG. 19A). Moreover, comparable results as with the ACD-A BCTs at 3 and 24 hrs were obtained (see FIG. 19B).

### 7. Example 7: Compatibility Testing of PAXgene Blood ccfDNA Tube with Parsortix Device for CTC Enrichment in Context of all-from-One Solution

In the context of Parsortix-based CTC (spiked tumor cells as a spike-in model) detection which was already tested in Example 2, we further evaluated the following options:

A. Detection of tumor cells based on immunofluorescent detection of tumor cells—staining of epithelial tumor-specific antigens.

B. Detection of spiked tumor cells based on their transcriptomic signatures (RT-PCR via AdnaTest AR-V7 panel).

For further information on the Parsonix device and the associated materials and methods we refer to Example 2 for conciseness.

### A. Detection of Tumor Cells Based on Immunofluorescent (IF) Detection of Tumor Cells—Staining of Epithelial Tumor-Specific Antigens

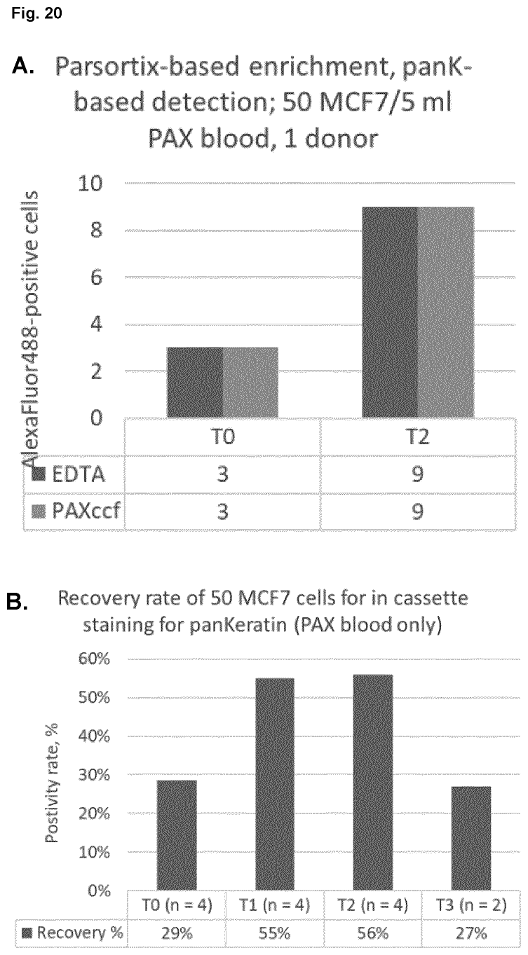

The Parsortix instrument (Angle PLC) offers two modes for quantitative (IF-based) detection of tumor cells. After the CTC enrichment program is done, the CTC enriched fraction can be harvested and is supplied as approx. 100 μl concentrate. This concentrate is placed on microscopy slides for further IF staining and microscopic evaluation. Alternatively, antibody staining can be performed in the separation cassette directly. The later approach is more efficient as diminishes potential losses of CTCs due to harvesting, centrifugation and staining steps. Spiked tumor cells (50 MCF7 cells) were detected via immunofluorescent staining of pan-cytokeratin either after harvest of CTC-enriched fraction or in-cassette staining (see FIGS. 20A and 20B, respectively). Storage of spiked blood has no impact on stainability of the cells (neither for in cassette staining nor for harvested cells). Spiked tumor cells seem to be stainable without any restrictions (see FIG. 21). Hence, the cells can be easily enriched and stained and therefore, are useful for the multimodal workflows described herein.

B. Detection of Spiked Tumor Cells Based on their Transcriptomic Signatures (RT-PCR Via AdnaTest AR-V7 Panel)

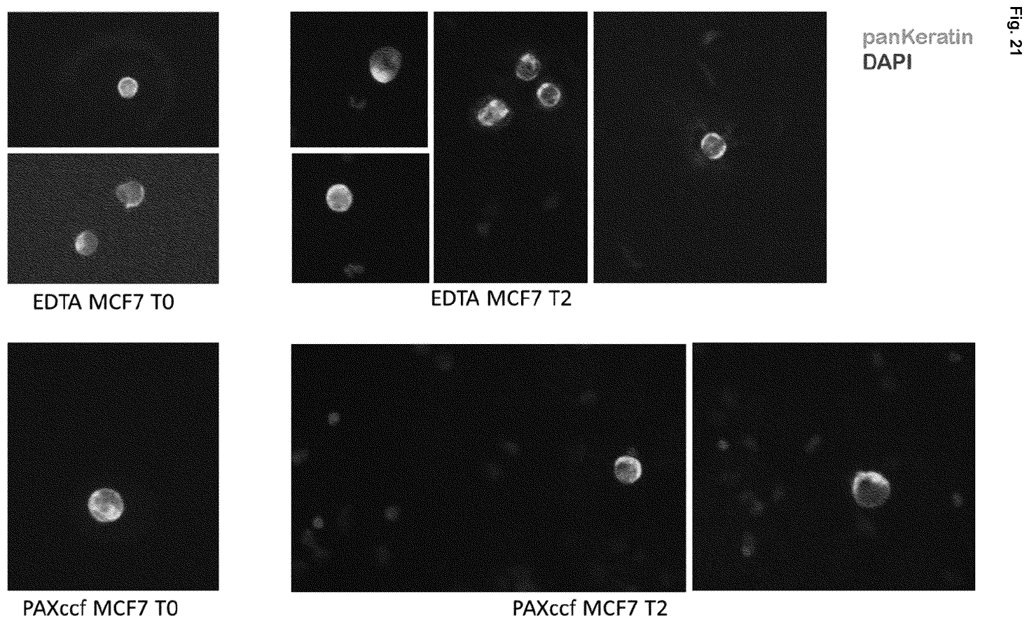

Alternatively to IF staining, enriched tumor cells can be detected based on their transcriptomic signatures. Therefore, enriched CTCs were harvested after Parsortix runs and detected using AdnaTest ProstateCancerPanel AR-V7 (only detection part) described above to which here is referred. As indicated in FIG. 22, cells spiked into PAX ccfDNA-collected blood samples and stored up to 3 days (TTP indicates the number of days) could be detected as efficiently as if spiked into EDTA-collected samples. These data underline compatibility of the PAXgene Blood ccfDNA Tubes with the Parsortix instrument for enrichment of CTCs either via IF staining of RT-PCR based assays.

### 8. Example 8: Multimodal Analysis of Circulating Cell-Free RNA (ccfRNA), Circulating Cell-Free DNA (ccfDNA) and Genomic DNA (gDNA) from Blood Samples Collected in PAXgene Blood ccfDNA Tubes

Besides circulating cell-free DNA (ccfDNA) from blood, also circulating cell-free RNA (ccfRNA) has gained relevance for biomarker studies. Combined insights from both analytes promise to increase the understanding of underlying molecular processes. Example 8 demonstrates the multimodal extraction and analysis of ccfRNA, ccfDNA and gDNA from one blood sample collected using the PAXgene® blood ccfDNA tube, which provides an advantageous stabilizing composition according to the present invention.

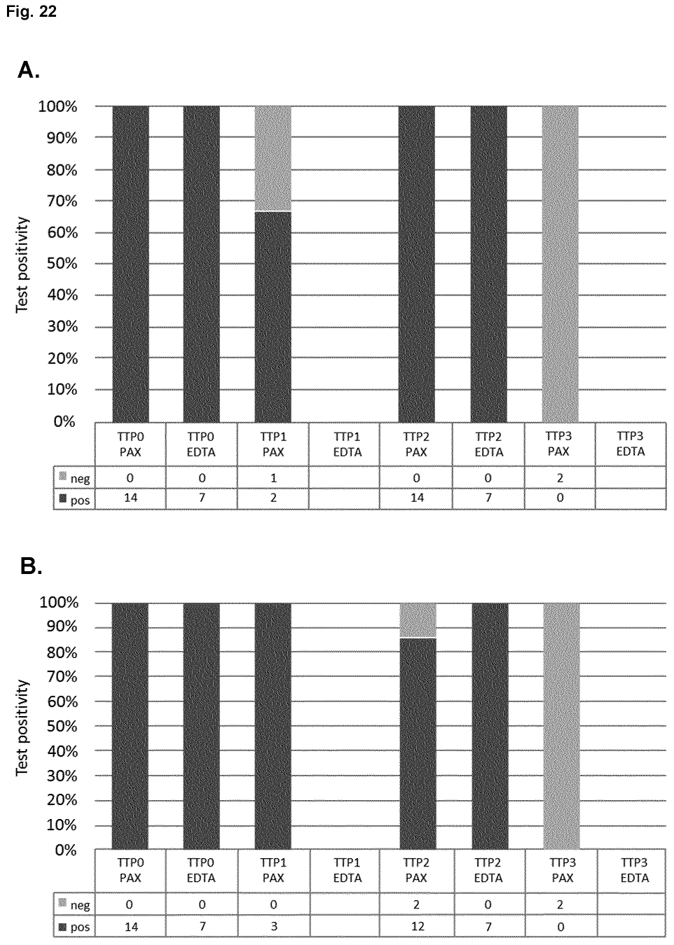

Whole blood samples were collected from healthy consented donors into PAXgene blood ccfDNA tubes (PreAnalytiX), BD Vacutainer® K₂EDTA tubes (BD), cell-free DNA BCT® (Streck®), RNA Complete BCT™ (Streck) and LBgard® blood tubes (Biomatrica). Plasma was generated by double centrifugation immediately after blood collection or after storage for up to three days. Cell-free nucleic acids were extracted as shown in FIG. 23.

### Results

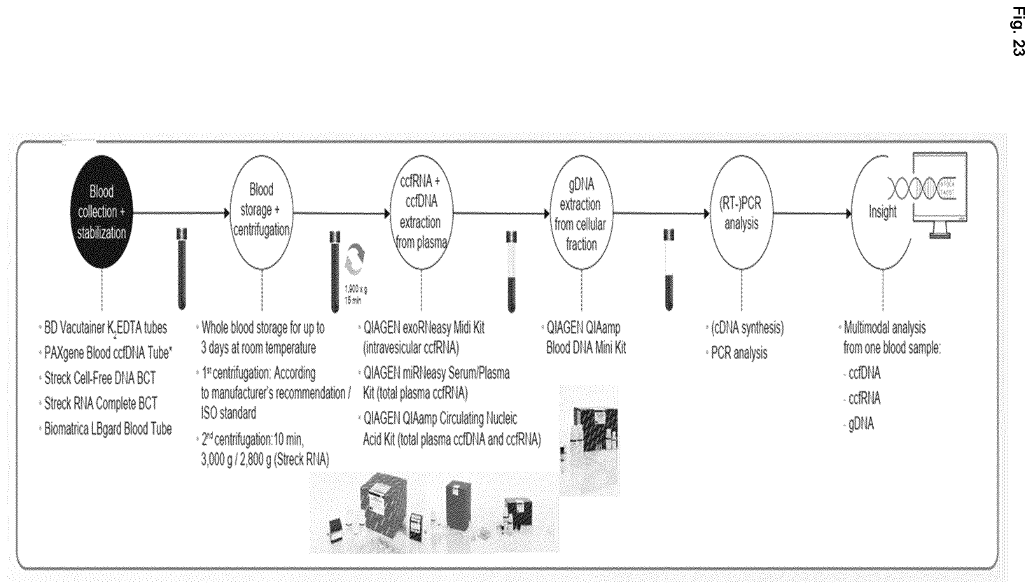

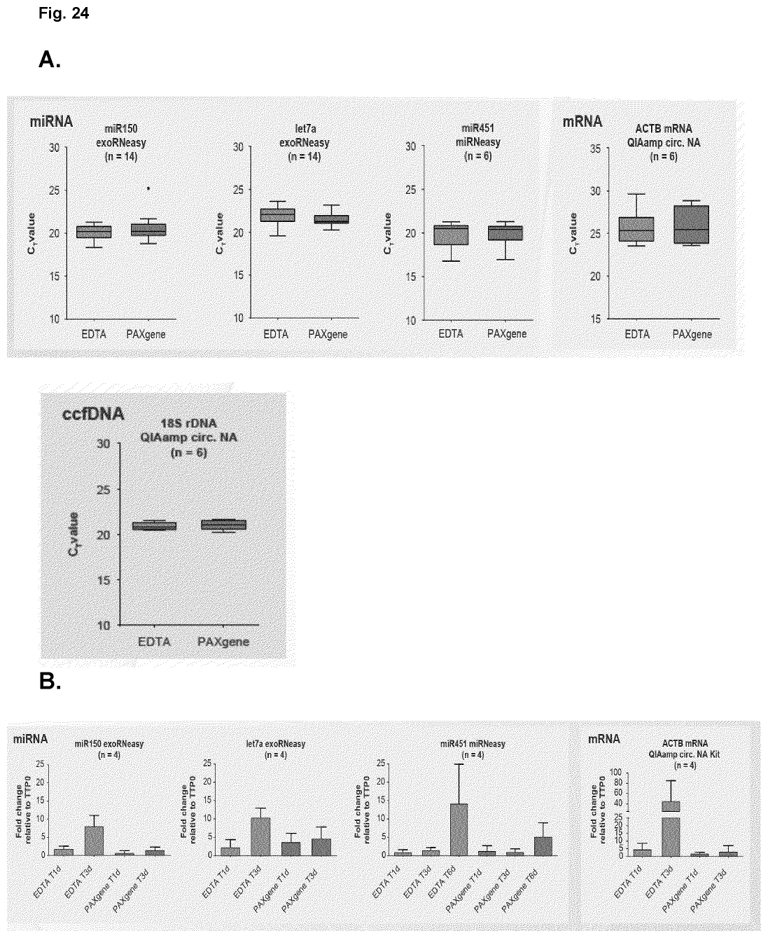

ccfRNA yield in plasma after blood storage in EDTA and PAXgene blood ccfDNA tubes is shown in FIG. 24A (comparison at TTP0) and FIG. 24B (relative fold change upon whole blood storage). The quantitative PCR analysis revealed comparable yields of miRNA, mRNA and ccfDNA targets from plasma of blood collected in PAXgene blood ccfDNA tubes and EDTA tubes. After blood storage in PAXgene blood ccfDNA tubes for up to three days, RNA targets (both intra- and extravesicular extracted with exoRNeasy and miRNeasy, respectively) could still be detected with improved stabilization over ETDA.

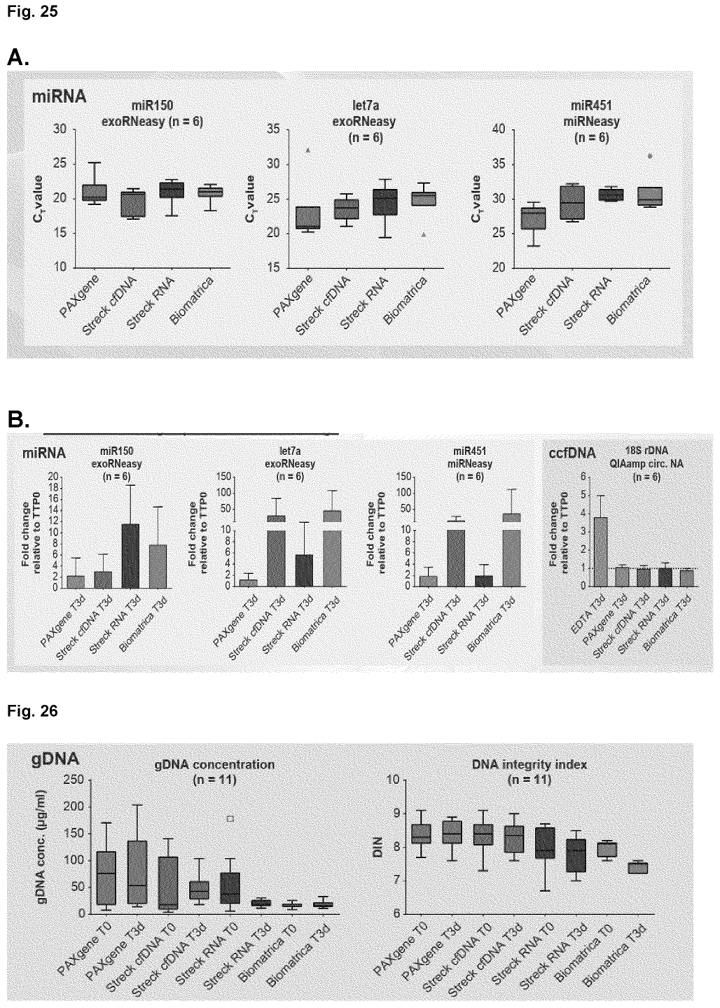

miRNA yield in plasma after blood storage in stabilization tubes is shown in FIG. 25A (comparison at TTP0) and FIG. 25B (relative fold change upon whole blood storage). RNA extraction and detection sensitivity was impacted by blood collection tubes containing formaldehyde-releasing formulations (Streck and Biomatrica) as indicated by higher C_{T} values at TTP0 (day 0) and lower RNA stabilization efficiency after 3 days of storage.

Genomic DNA yield and integrity is shown in FIG. 26. The PAXgene blood ccfDNA tubes furthermore enabled efficient gDNA extraction from residual blood cells after plasma separation following 3 days of whole blood storage with intact DNA as indicated by stable DNA integrity index. In contrast, gDNA yield and integrity were reduced by collection and storage in Streck RNA and Biomatrica tubes.

The results provided by the multimodal analysis of Example 8 further demonstrate that the non-crosslinking technology of the stabilization composition of the present invention is highly advantageous enables the isolation and analysis of cell-free miRNA, mRNA, ccfDNA and furthermore genomic cellular gDNA from a single sample. In addition and as demonstrated by the other examples, further rare cell populations such as CTCs can be enriched and detected. The data overall demonstrates that the present invention provides an advantageous multimodal workflow that is highly useful in liquid biopsy research.

Other stabilization technologies showed impaired analysis efficiency after whole blood storage for the tested targets of interest as is demonstrated by the multiple examples contained herein.

1-27. (canceled)

28. A method for stabilizing and enriching multiple biological targets comprised in a cell-containing bodily fluid, said method comprising:
(A) contacting a cell-containing bodily fluid with a stabilizing composition comprising one or more of the stabilizing agents selected from the group consisting of
(a) a primary, secondary or tertiary amide,
(b) a poly(oxyethylene) polymer, and
(c) a apoptosis inhibitor,
thereby providing a stabilized cell-containing bodily fluid sample;
(B) keeping the stabilized cell-containing bodily fluid sample for a stabilization period; and
(C) processing the stabilized cell-containing bodily fluid sample in order to enrich three or more biological targets selected from the group consisting of
a cell subpopulation,
extracellular nucleic acids,
extracellular vesicles, and
intracellular nucleic acids
from the stabilized cell-containing bodily fluid and thereby obtain an enriched cell population.

29. The method according to claim 28, wherein the enriched cell subpopulation comprises target rare cells, optionally wherein the target rare cells are selected from the group consisting of circulating tumor cells (CTCs), fetal cells, stem cells, cells infected by a virus or parasite, circulating endothelial cells (CECs) and circulating endothelial progenitor cells (EPCs).

30. The method according to claim 28, wherein step (C) comprises obtaining at least one cell-containing fraction and at least one cell-depleted fraction from the stabilized bodily fluid sample, wherein the processing in (C) comprises performing steps set forth in one of variant A, variant B and variant C, and wherein
variant A comprises:
(aa) separating the stabilized cell-containing bodily fluid sample into at least one cell-containing fraction and at least one cell-depleted fraction;
(bb) further processing the cell-containing fraction, wherein further processing the cell-containing fraction comprises
(i) enriching a cell subpopulation from the cell-containing fraction; and/or
(ii) enriching intracellular nucleic acids from the cell-containing fraction;
(cc) further processing the cell-depleted fraction, wherein further processing the cell-depleted fraction comprises
(i) enriching extracellular nucleic acids, optionally extracellular DNA, from the cell-depleted fraction; and/or
(ii) enriching extracellular vesicles from the cell-depleted fraction;
variant B comprises:
(aa) enriching a cell subpopulation from the stabilized cell-containing bodily fluid sample;
(bb) separating the stabilized cell-containing bodily fluid sample from which the target cell subpopulation was removed into a cell-containing fraction and a cell-depleted fraction;
(cc) further processing the cell-depleted fraction, wherein further processing the cell-depleted fraction comprises
(i) enriching extracellular nucleic acids, optionally extracellular DNA, from the cell-depleted fraction; and/or
(ii) enriching extracellular vesicles from the cell-depleted fraction; and
(dd) optionally enriching intracellular nucleic acids from the cell-containing fraction; and
variant C comprises:
(aa) dividing the stabilized cell-containing bodily fluid sample into at least two aliquots and enriching a cell subpopulation from at least one of the provided aliquots;
(bb) providing at least one cell-containing fraction and at least one cell-depleted fraction;
(cc) further processing the cell-depleted fraction, wherein further processing the cell-depleted fraction comprises
(i) enriching extracellular nucleic acids, optionally extracellular DNA, from the cell-depleted fraction; and/or
(ii) enriching extracellular vesicles from the cell-depleted fraction; and
(dd) optionally enriching intracellular nucleic acids from the cell-containing fraction.

31. The method according to claim 28, further comprising:
(D) processing the enriched three or more biological targets for analysis.

32. The method according to claim 31, having one or more of the following characteristics:
(i) step (C) comprises enriching target rare cells and subsequent step (D) comprises analysing the enriched target rare cells on a cellular level and/or by isolating intracellular nucleic acids from the enriched target rare cells and detecting one or more target molecules within the isolated intracellular nucleic acids, optionally wherein the intracellular nucleic acid comprises mRNA;
(ii) step (C) comprises obtaining a cell-depleted fraction from the stabilized cell-containing bodily fluid sample and isolating extracellular nucleic acids from the obtained cell-depleted fraction, optionally wherein the extracellular nucleic acids comprise or essentially consist of extracellular DNA, and subsequent step (D) comprises detecting one or more target molecules within the isolated extracellular nucleic acids;
(iii) step (C) comprises enriching extracellular vesicles from a cell-depleted fraction obtained from the stabilized cell-containing bodily fluid sample and subsequent step (D) comprises isolating RNA from the enriched extracellular vesicles and detecting one or more target molecules within the isolated RNA; and
(iv) step (C) comprises isolating as biological targets at least (i) circulating tumor cells, (ii) genomic DNA and (iii) circulating cell-free DNA and wherein step (D) comprises (i) isolating RNA from the circulating tumor cells and detecting biomarker RNA molecules in the isolated RNA; (ii) detecting, e.g. amplifying and/or sequencing, genomic DNA and (iii) detecting biomarker molecules in the isolated circulating cell-free DNA.

33. The method according to claim 28, comprising enriching target rare cells and/or extracellular vesicles by affinity capture.

34. The method according to claim 28, wherein the cell-containing bodily fluid has one or more of the following characteristics:
it is a circulating bodily fluid;
it is selected from blood, urine, saliva, synovial fluids, amniotic fluid, lachrymal fluid, lymphatic fluid, liquor, cerebrospinal fluid, sweat, ascites, milk, bronchial lavage, peritoneal effusions and pleural effusions, bone marrow aspirates and nipple aspirates, semen/seminal fluid, body secretions or body excretions;
it is selected from blood and urine; and
it is blood.

35. The method according to claim 28, wherein the stabilization composition comprises at least one primary, secondary or tertiary amide and wherein the stabilizing composition comprises at least one primary, secondary or tertiary amide according to formula 1: 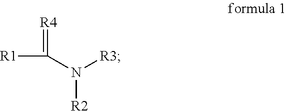
wherein R1 is a hydrogen residue or an alkyl residue, R2 and R3 are identical or different and are selected from a hydrogen residue and a hydrocarbon residue, and R4 is an oxygen, sulphur or selenium residue, and, optionally, wherein the at least one compound according to formula 1 is a primary, secondary or tertiary carboxylic acid amide, N,N-dimethlypropanamide, butanamide or another N,N-dialkylpropanamide.

36. The method according to claim 28, wherein the stabilization composition comprises at least one poly(oxyethylene) polymer, optionally wherein the poly(oxyethylene) polymer is a polyethylene glycol.

37. The method according to claim 36, wherein the stabilizing composition has one or more of the following characteristics:
a) the comprised poly(oxyethylene) polymer is an unsubstituted polyethylene glycol;
b) the composition comprises a poly(oxyethylene) polymer which is a high molecular weight poly(oxyethylene) polymer having a molecular weight of at least 1500;
c) the composition comprises at least one poly(oxyethylene) polymer having a molecular weight below 1500, optionally wherein the molecular weight lies in a range selected from 100 to 1000, 200 to 800, 200 to 600 and 200 to 500;
d) the composition comprises a poly(oxyethylene) polymer which is a high molecular weight poly(oxyethylene) polymer having a molecular weight of at least 1500 and comprises a low molecular weight poly(oxyethylene) polymer having a molecular weight of 1000 or less; and
e) the composition comprises a poly(oxyethylene) polymer which is a high molecular weight poly(oxyethylene) polymer and a poly(oxyethylene) polymer which is a low molecular weight poly(oxyethylene) polymer having a molecular weight of 1000 or less, wherein said high molecular weight poly(oxyethylene) polymer has a molecular weight that lies in a range selected from 1500 to 50000, 2000 to 40000, 3000 to 30000, 3000 to 25000, 3000 to 20000 and 4000 to 15000 and/or wherein said low molecular weight poly(oxyethylene) polymer has a molecular weight that lies in a range selected from 100 to 1000, 200 to 800, 200 to 600 and 200 to 500.

38. The method according to claim 28, wherein the stabilization composition comprises at least one caspase inhibitor as apoptosis inhibitor, optionally wherein the caspase inhibitor has one or more of the following characteristics:
a) the caspase inhibitor is a pancaspase inhibitor;
b) the caspase inhibitor comprises a caspase-specific peptide;
c) the caspase inhibitor comprises a modified caspase-specific peptide that is modified with an O-Phenoxy (OPh) group;
d) the caspase inhibitor comprises a modified caspase-specific peptide that is modified with a glutamine (Q) group;
e) the caspase inhibitor is selected from the group consisting of Q-VD-OPh, Boc-D-(OMe)-FMK and Z-Val-Ala-Asp(OMe)-FMK;
f) the caspase inhibitor is selected from the group consisting of Q-VD-OPh and Z-Val-Ala-Asp(OMe)-FMK; and
g) the caspase inhibitor is Q-VD-OPh.

39. The method according to claim 28, wherein the stabilizing composition comprises:
(a) at least one primary, secondary or tertiary amide;
(b) at least one poly(oxyethylene) polymer; and
(c) at least one caspase inhibitor; and
(d) optionally EDTA and/or another chelating agent.

40. The method according to claim 35, wherein the cell-containing bodily fluid is blood and the blood is contacted with:
a) one or more compounds according to formula 1;
b) at least one high molecular weight poly(oxyethylene) polymer having a molecular weight that lies in a range of 3000 to 40000 and at least one low molecular weight poly(oxyethylene) polymer having a molecular weight of 1000 or less;
c) at least one caspase inhibitor, preferably a pancaspase inhibitor, optionally Q-VD-OPh; and
d) an anticoagulant which optionally is EDTA or another chelating agent,
wherein after the blood sample has been contacted with said additives and optionally further additives used for stabilization the resulting mixture/stabilized blood sample comprises
the one or more compounds according to formula 1 in a concentration that lies in a range of 0.3% to 4%,
the high molecular weight poly(oxyethylene) polymer in a concentration that lies in a range of 0.2% to 1.5% (w/v),
the low molecular weight poly(oxyethylene) polymer in a concentration that lies in the range of 1.5% to 10%, and
the caspase inhibitor in a concentration that lies in a range of 1 μM to 10 μM.

41. The method according to claim 28, having one or more of the following characteristics:
(i) the stabilization of the cell-containing body fluid sample does not involve the use of additives in a concentration wherein said additives would induce or promote lysis of nucleated cells;
(ii) the stabilization does not induce protein-nucleic acids or protein-protein cross-links;
(iii) the stabilization does not involve the use of a cross-linking agent that induces protein-nucleic acid and/or protein-protein crosslinks;
(iv) the stabilization does not involve the use of toxic agents; and
(v) the stabilizing agents are contained in an stabilization composition comprising water.

42. The method according to claim 28, wherein the stabilization used in step (A) does not induce protein-nucleic acids or protein-protein cross-links in the stabilized sample, optionally wherein step (C) comprises enriching extracellular vesicles from a cell-depleted fraction obtained from the stabilized cell-containing bodily fluid sample and subsequent step (D) comprises isolating RNA from the enriched extracellular vesicles and detecting one or more target molecules within the isolated RNA.

43. The method according to claim 41, wherein the cell-containing bodily fluid, preferably blood, is contacted with:
a) one or more compounds according to formula 1;
b) at least one high molecular weight poly(oxyethylene) polymer having a molecular weight of at least 3000 and optionally at least one low molecular weight poly(oxyethylene) polymer having a molecular weight of 1000 or less;
c) at least one caspase inhibitor; and
d) optionally EDTA or another chelating agent.

44. The method according to claim 41, wherein step (C) comprises processing the stabilized cell-containing bodily fluid sample in order to enrich three or more biological targets selected from the group consisting of rare cells,
extracellular nucleic acids,
extracellular vesicles, and
intracellular nucleic acids,
from the stabilized cell-containing bodily fluid.

45. The method according to claim 41, wherein step (C) comprises obtaining at least one cell-containing fraction and at least one cell-depleted fraction from the stabilized bodily fluid sample and wherein step (C) further comprises enriching extracellular vesicles from the cell-depleted fraction obtained from the stabilized cell-containing bodily fluid sample and subsequent step (D) comprises isolating RNA from the enriched extracellular vesicles.

46. The method according to claim 45, wherein step (D) comprises detecting one or more target molecules within the isolated RNA.

47. The method according to claim 45, comprising isolating genomic DNA from the cell-containing fraction.

48. The method according to 28, wherein processing step (C) comprises subjecting the stabilized cell-containing bodily fluid sample or a cell-containing fraction obtained from the stabilized cell-containing bodily fluid sample to a density gradient centrifugation step, optionally wherein the cell-containing bodily fluid sample is blood.

49. The method according to claim 48, wherein a stabilized blood sample or a cell-containing fraction obtained from the stabilized blood sample is diluted with a dilution solution prior to performing the density gradient centrifugation step.

50. The method according to claim 49, wherein the dilution solution has one or more of the following characteristics:
(a) it is a hypotonic solution or an isotonic solution;
(b) it comprises a tonicity modifier;
(c) it comprises a polyol, optionally a sugar or sugar alcohol;
(d) it comprises a sugar, optionally glucose;
(e) it comprises a sugar alcohol, optionally glycerol; and
(f) it comprises a salt, optionally an alkali metal salt, optionally a chloride salt; and
wherein after density gradient centrifugation, different layers are formed, wherein the formed layers comprise a PBMC layer.

51. The method according to claim 50, wherein
(a) the dilution solution comprises a reducing sugar, optionally glucose, in a concentration that lies in a range of 2-10%, 3-7% or 4-6% (w/v);
(b) the dilution solution comprises a sugar alcohol and a salt, optionally wherein the dilution solution comprises up to 0.5M glycerol and up to 2% sodium chloride,
(c) the dilution solution comprises 0.7-1.2% sodium chloride and 0.075-0.15M glycerol, and/or
(d) wherein the dilution solution is selected from
(i) 5% (w/v) glucose,
(ii) 0.9% NaCl+0.1 M glycerol, and
(iii) a dilution solution comprising at least one tonicity modifier and having a osmolality that corresponds to the osmolality of the dilution solution defined in (i) or (ii), or wherein the osmolality is within a range of +/−20%, +/−15% or +/−10% of the osmolality of the solution as defined in (i) or (ii).

52. The method according to claim 49, wherein the dilution solution comprises DMSO.
